(19) United States
(12) Patent Application Publication
(10) Pub. No.: US 2024/0116896 A1
(43) Pub. Date: Apr. 11, 2024

(54) HETEROATOM SUBSTITUTED CYCLIC AND ALKYL AMINES AS ACTIVATORS OF SEROTONIN RECEPTORS

(71) Applicant: ATAI Life Sciences AG, Berlin (DE)

(72) Inventors: Tanweer A. KHAN, Bridgewater, NJ (US); Alan C. GIBBS, Wyndmoor, PA (US); Glenn SHORT, Scituate, MA (US); Robert B. PERNI, Marlborough, MA (US)

(21) Appl. No.: 18/462,120

(22) Filed: Sep. 6, 2023

Related U.S. Application Data
(60) Provisional application No. 63/404,082, filed on Sep. 6, 2022.
(60) Provisional application No. 63/503,335, filed on May 19, 2023.

(51) Int. Cl. C07D 403/06; A61P 25/00; C07D 209/14; C07D 471/04; C07F 9/572

(52) U.S. Cl. CPC C07D 403/06; A61P 25/00; C07D 209/14; C07D 471/04; C07F 9/5728

(57) ABSTRACT

The present disclosure provides N,N-dimethyltryptamine (N,N-DMT) derivatives of the present disclosure provides compounds of formula (I), (I-A), (I-B), (I-C), (II), (II-A), (III), and (IV), or pharmaceutically acceptable salts or deuterated forms thereof. Also provided herein are methods of using the N,N-DMT derivatives, e.g., in the treatment of mental health diseases and/or disorders.

### CROSS-REFERENCE TO RELATED APPLICATIONS

The present application claims the benefit to U.S. Provisional Application No. 63/404,082, Sep. 6, 2022, and U.S. Provisional Application No. 63/503,335, May 19, 2023, the contents of which are hereby incorporated by reference in their entirety.

### BACKGROUND OF THE INVENTION

### Field of Invention

The present disclosure relates to compounds for treating mental health disorders which overcome solubility and oral bioavailability issues associated with DMT, F-HO-DMT or bufotenine, 5-MeO DMT or O-methyl-bufotenin and psilocin. The present disclosure also relates to compounds which act as (partial) agonists of the CNS serotonin receptor, 5-HT2A, including modified psychedelics which do not cause a hallucinogenic effect.

### Description of Related Art

G protein-coupled receptors, or GPCRs, are a major class of membrane proteins. Approximately 800 different GPCRs are encoded by the human genome and when expressed are located in the plasma membrane to act as the ‘eyes and ears’ of the cell (Gurevich and Gurevich, 2019). Structurally they are composed of seven transmembrane alpha helices connected by intra- and inter-cellular loops of various lengths. These helices and loops play important roles in binding effectors, and/or other proteins, which often results in a signaling or communication event. Signaling of GPCRs produce cellular responses crucial for the health and benefit of the cell and organism.

Several GPCRs are expressed in the central nervous system (CNS), one example is the serotonin family of receptors. The serotonin family is divided into subfamilies, 5-HT1 to 5-HT7 (note: 5-HT3 is a non-GPCR subfamily) and further into subtypes, eg: the 5-HT2 subfamily is composed of 5-HT2A, 5-HT2B and 5-HT2C (Pandy-Szekeres, G. et. al., 2022). 12 serotonin GPCR subtypes have been identified. Serotonin receptors bind serotonin (or 5-hydroxytryptamine) triggering signal transduction, the downstream effects of which modulate a variety of processes such as: memory, sleep, mood and vision among others (Sizemore, T. R., et. al., 2020). In addition to serotonin, serotonin receptors are known to bind other endogenous neurotransmitters as well as exogenous small molecules. Indeed, many small molecule drugs have been developed that either activate or deactivate serotonin receptors leading to positive outcomes for a variety of neuropsychiatric disorders (Terry, A. V., 2004).

Such compounds include classic psychedelic tryptamines including N, N-dimethyltryptamine (DMT), 5-methoxy-DMT (5-MeO-DMT) and psilocybin (specifically psilocin or 4-hydroxy-DMT) bind select serotonin receptors in the active state and are known to be agonists or partial agonists (McClure-Begley, T. D and Roth, B. L., 2022). These compounds have attracted increasing attention as they are thought to be therapeutically efficacious for various mental health disorders such as MDD, TRD, SUD, as well as compulsive, anxiety, stress and eating disorders (Mertens, L. J. and Preller, K. H., 2021).

However, many classic psychedelic tryptamines suffer from poor oral bioavailability due to metabolism by monoamine oxidases (MAOs) and are therefore not suitable as oral therapeutic agents. For example, a strong first pass effect oxidizes 100% of DMT after oral administration and therefore DMT must be administered with a MOA-A inhibitor to be orally active (Riba, J., et. al., 2015). There continues to be a need, therefore, for psychedelic tryptamines which are bioavailable orally.

Furthermore, classic psychedelic tryptamine solubility and duration of target engagement may not be ideal for many therapeutic purposes due to their potential for abuse. In addition to bioavailability concerns, it is well known that tryptamines suffer from the significant drawback of being abused due to their psychedelic and hallucinogenic effects.

The question has been raised whether it is possible to achieve the desirable therapeutic effects of psychedelic tryptamines without the undesired hallucinogenic effects which have been the source of their potential for abuse. In this regard, the results have to date been inconclusive. As reported by McClure-Begley, it is currently unknown whether the subjective experience of a psychedelic drug is a necessary component of its ability to produce therapeutic benefits. Thus, currently it is difficult to state with any certainty anything beyond the existence of a relationship between these observations. Similarly, Olson has reported that although preliminary evidence suggests that the subjective effects of psychedelics are not necessary to produce therapeutic responses, they may be critical for achieving maximal efficacy. Olson proposed three possible solutions namely (1) use of micro doses of the psychedelic; (2) design of analog compounds which lack psychedelic properties and (3) using anesthesia on a patient while administering the psychedelic.

Olson found that administration of low, sub hallucinogenic doses of psychedelics have the potential to shed light on the role of mystical type experiences in therapeutic responses. Though anecdotal reports suggest that psychedelic micro dosing—the chronic, intermittent use of sub hallucinogenic doses—may produce beneficial effects and relieve symptoms of depression and anxiety, Olson suggested interpreting these results with caution given the nature of the testing done. Olson further reported demonstrating that psychedelic microdosing produced antidepressant-like and anxiolytic effects in rodents with minimal to no impact on other behavioral measures but noted that such results have not yet been confirmed in humans. Olson cautioned that the low doses required to avoid significant subjective effects may simply be insufficient to activate 5-HT2A receptors to produce long-lasting changes in neural circuitry.

Olson also proposed that the development of nonhallucinogenic compounds capable of producing psychedelic-like therapeutic effects would solve these issues and greatly improve patient access. Olson reported transposition of the N,N-dimethylaminoethyl group of 5-methoxy-N,N-dimethyltryptamine (5-MeO-DMT) from the C3 to the N1 position of the indole to yield 6-MeO-isoDMT. Olson reported that this compound exhibits significantly reduced hallucinogenic potential, as measured by the mouse head-twitch response (HTR) assay, while retaining psychoplastogenic potency comparable to its hallucinogenic congener. Olson further noted many of the nonhallucinogenic analogues of psychedelics that his group developed will not produce hallucinogenic behavioral responses in rodents even at extremely high doses. Noting that it was unknown if nonhallucinogenic psychoplastogens could produce beneficial behavioral effects comparable to psychedelics, Olson reported that through careful chemical design, his group engineered tabernanthalog (TBG), a nonhallucinogenic analogue of 5-MeO-DMT that promotes cortical neuron structural plasticity through activation of 5-HT2A receptors. Like psychedelic compounds, TBG was said to demonstrate preclinical therapeutic effects suggesting that it might be effective at treating a range of neuropsychiatric diseases including depression, alcohol use disorder, and heroin use disorder, although Olson noted that future work would need to address why functionally selective 5-HT2A receptor ligands such as TBG can produce plasticity and therapeutic behavioral responses without inducing behavioral effects characteristic of classic psychedelics. Ultimately, clinical trials will be necessary to determine if psychoplastogenic analogues of psychedelics can produce therapeutic effects in humans without inducing mystical-like experiences.

Olson concluded that, ultimately, clinical trials will be necessary to determine if psychoplastogenic analogues of psychedelics can produce therapeutic effects in humans without inducing mystical-like experiences. Olson also discussed the potential of psychedelic microdosing to produce beneficial effects and relieve symptoms of depression and anxiety. Olson postulated, however, that the low doses required to avoid significant subjective effects may simply be insufficient to activate 5-HT2A receptors to produce long-lasting changes in neural circuitry. Despite the promising therapeutic responses produced by psychedelic-assisted therapy, Olson concluded that the intense subjective effects of these drugs make it unlikely that they will ever become widespread treatments for disorders such as depression.

Dunlap et al. (2020) have reported that dimethyltryptamine (DMT), which is the core feature of many psychedelic compounds, can be engineered to lack hallucinogenic potential while retaining the ability to promote neural plasticity, identifying key features of the “psychoplastogenic pharmacophore” to develop psychoplastogens that are easier to make, have improved physicochemical properties (stability, metabolic profiles), and show a reduced or absent hallucinogenic potential compared to DMT. Citing the earlier work of Glennon et al., Dunlap et al. reported addressing this issue by transposing the N1 and C3 atoms of DMT to produce a small series of N,N-dimethylaminoisotryptamine (isoDMT), analogs with reduced hallucinogenic potential as measured by their abilities to substitute for known hallucinogens in rodent drug discrimination assays. Dunlap et al. postulate that, in principle, related analogs could be accessed in a single step through N-alkylation of the corresponding indoles or related heterocycles. Additionally, Dunlap et al. purported to demonstrate the possession by several isoDMTs of a comparable affinity for serotonin receptors as compared to their DMT counterparts. Dunlap et al. reported that the 5-HT2A receptor is necessary for the psychoplastogenic effects of DMT and, as isoDMTs are known to bind to 5-HT2A receptors, hypothesized that isoDMT analogs would still be capable of promoting neuronal growth despite lacking indole N—H bonds. Furthermore, isoDMT analogs are likely to exhibit improved physicochemical properties as the loss of a hydrogen bond donor decreases total polar surface area and improves central nervous system multiparameter optimization (MPO) scores.

Dunlap et al. concluded that isoDMT derivatives with low hallucinogenic potential are capable of promoting dendritogenesis to a comparable extent as the psychedelic DMT and the state-of-the-art fast-acting antidepressant ketamine. Dunlap et al.'s SAR studies defined the minimal psychoplastogen pharmacophore as an aromatic ring separated from a basic nitrogen by a short linker. Dunlap et al. also reported that substitution at the 4-position of isoDMT derivatives renders them devoid of psychoplastogenic properties, thus demonstrating that a psychedelic compound (i.e., DMT) can be engineered to lack hallucinogenic potential while retaining the ability to promote neural plasticity.

Soft drugs (SDs) are therapeutically active compounds that undergo a predicted fast metabolism into inactive metabolites after exerting their desired therapeutic effects. Buckwald, P. Soft drugs: design principles, success stories, and future perspectives. Expert opinion on drug metabolism & toxicology, 16(8), 645 (2020). The goal of SD design is to control and direct metabolism, typically by incorporation of a metabolically sensitive moiety into the structure. The SD concept is part of the more general recognition that drug design needs to (1) fully integrate metabolic considerations from the very beginning as metabolites contribute significantly to the overall activity and toxicity profile of the original drug; and (2) focus not on improving activity alone, but on improving the activity/toxicity ratio. For most drugs, several metabolites are formed following administration, and they can contribute significantly not just to the overall activity, but also to toxicity and side effects. For SDs, inactivation should be relatively fast and free of interference from possible drug-drug interactions. SDs should not be confused with prodrugs, mainly because (1) both undergo metabolic changes and (2) both rely primarily on enzymatic hydrolysis. SDs, however, are active per se and are inactivated by a built-in mechanism, whereas prodrugs are inactive and must be activated.

As reported by Buckwald, development of soft drugs faces a number of significant challenges, such as the need to achieve an adequate balance between maximizing the desired local activity and minimizing the undesired systemic toxicity. Further, SDs have to be sufficiently stable to reach their intended targets/receptors and produce their desired effects while remaining sufficiently fragile to not cause unwanted systemic side effects. Several SD designs failed in the end because the metabolic degradation was too fast and acceptable activity could not be achieved. For ester-containing drugs, including SDs and prodrugs, a further challenge is that esterase activities vary strongly among species as well as among organs and tissues.

### SUMMARY OF THE INVENTION

Aspects of the present disclosure provide compounds of Formula (I-C),

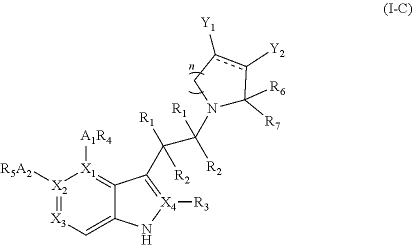

or a pharmaceutically acceptable salt or deuterated form thereof;
wherein,
Y₁ and Y₂ are each independently hydrogen (H), deuterium (D), —CH₃, —OR₁, —CO₂R₈, —NR₁₀R₁₁;
X₁, X₂, X₃, or X₄ are each independently —CH, —C, or —N;
A₁ and A₂ are each independently a bond, H, D, O, N, or S;
R₁, R₂, and R₃ are each independently H, D, alkyl, cycloalkyl, aromatic, halogen, heteroaromatic, or absent;
R₄ and R₅ are each independently H, D, alkyl, cycloalkyl, aromatic, halogen substituted alkyl or cycloalkyl, halogen, heteroaromatic, —C(O)CH₃, or —P(O)(OR₉)₂;
R₆ is H or alkyl;
R₇ is

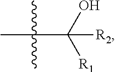

—CO₂R₈, —CH₂C(O)CH₃, —CH₂CO₂R₈, —OP(O)(OR₉)₂, —C(O)—NR₁₀R₁₁;
R₈ is H, alkyl, cycloalkyl, haloalkyl, alkyl-O-alkyl, or aryl;
R₉ is H or alkyl;
R₁₀ and R₁₁ are each independently H or alkyl; and
n is 0, 1, or 2, wherein the compound is not

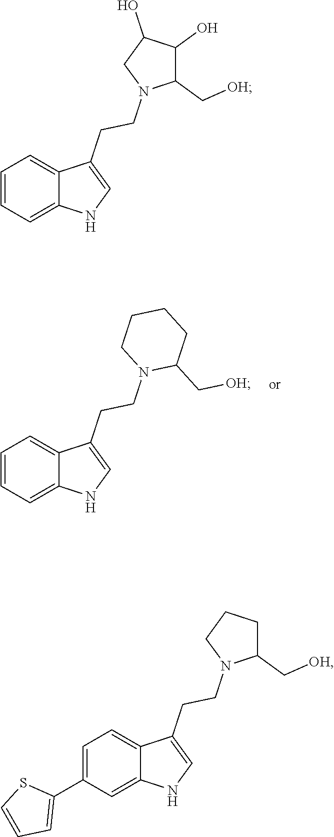 [SMILES: OCC1C(O)C(O)CN1CCc1c[nH]c2ccccc12 ; OCC1CCCCN1CCc1c[nH]c2ccccc12 ; OCC1CCCN1CCc1c[nH]c2cc(-c3cccs3)ccc12]

and wherein when n is 2, R⁷ is not —CO₂R₈ or —CH₂CO₂R₈.

### BRIEF DESCRIPTION OF THE DRAWINGS

FIGS. 1A-1D show in vitro data for compound 1.

FIG. 2 shows in vitro data for compound 2.

FIG. 3A-3D show in vitro data for compound 3.

FIG. 4 shows in vitro data for compound 4.

FIG. 5 shows in vitro data for compound 5.

FIG. 6 shows in vitro data for compound 6.

### DETAILED DESCRIPTION

In embodiments, the present disclosure provides DMT derivatives which are orally bioavailable. In embodiments, the present disclosure applies the soft drug concept by providing a DMT compound modified at the a nitrogen to include an ester-containing moiety which, in vivo, metabolizes to an inactive acid metabolite and additional hetero atom-containing side products such as alcohols or amines which are easily cleared from circulation via a second phase metabolism. Thus, disclosed herein are compounds with potential for treating mental health disorders which overcome stability, solubility, and oral bioavailability issue which preferably also act as (partial) agonists of the CNS serotonin receptor, 5-HT2A. These compounds persist through first pass metabolism and cross the blood brain barrier, resulting in therapeutically effective concentrations at the site of action. Moreover, compounds identified herein maintain overall selectivity profiles similar to that of DMT, 5-MeO-DMT and 4-hydroxy-DMT but with the ability to improve PK properties and half-life. In addition, because in an embodiment the compounds metabolize in vivo to inactive metabolites, they can further be designed so as to metabolize before causing a euphoric effect, thereby reducing their potential for abuse.

The present disclosure thus relates to, but is not limited to, both DMT compounds with greater oral bioavailability as well as to the so-called anti- or soft-drugs (Buchwald, P., 2020). The soft-drugs described herein are (partial) agonists in present form but are subsequently metabolized in vivo to inactive metabolites, in an appropriate time interval.

### Definitions

The terms “administer,” “administering” or “administration” as used herein refer to administering a compound or pharmaceutically acceptable salt of the compound or a composition or formulation comprising the compound or pharmaceutically acceptable salt of the compound to a patient.

In this specification, unless stated otherwise, the term “pharmaceutically acceptable” is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have.

Deleterious effects may include, for example, excessive toxicity, irritation, allergic response, and other problems and complications.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The term “treating” as used herein with regard to a patient, refers to improving at least one symptom of the patient's disorder. In embodiments, treating can be improving, or at least partially ameliorating a disorder or one or more symptoms of a disorder.

The term “preventing” as used herein with regard to a patient or subject, refers to preventing the onset of disease development if none had occurred, preventing the disease or disorder from occurring in a subject or a patient that may be predisposed to the disorder or disease but has not yet been diagnosed as having the disorder or disease, and/or preventing further disease/disorder development if already present.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

The term “substituted” used herein means any of the groups described herein (e.g., alkyl, alkenyl, alkynyl, alkoxy, aryl, cycloalkyl, cycloalkenyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —O R_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

### Compounds

In embodiments, the present disclosure provides compounds of formula (I), (I-A), (I-B), (I-C), (II), (II-A), (III), and (IV), or pharmaceutically acceptable salts or deuterated forms thereof.

In embodiments, the present disclosure provides compounds of formula (I):

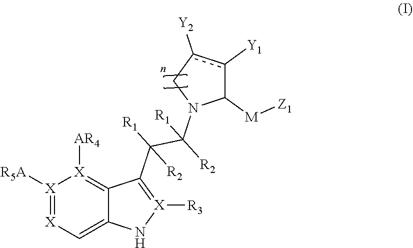

or a pharmaceutically acceptable salt thereof; wherein,
Y₁ and Y₂ are independently hydrogen/deuterium, —CH₂—, —OR₁, —CO₂, or —NR₁,R₂
Z₁ is independently —OH/OD, —SH/SD, OR₁ or —NR₁,R₂
A is H, D, O, N or S
X are independently —C, —N
M is CH₂, C═O, or CR₁R₂
R₁, R₂ and R₃ are independently H or D or alkyl, cycloalkyl, heteroatom substituted, and halogen substituted alkyl or cycloalkyl, aromatic or heteroaromatic
R₄ and R₅ are independently H or D or alkyl, cycloalkyl, heteroatom substituted, and halogen substituted alkyl or cycloalkyl, aromatic or heteroaromatic
or
R₄ and R₅ are prodrugs, like aliphatic, aromatic, heteroaromatic esters, aminal and hemiaminal, —CH₂—OPO₃H₂ or —CH alkyl-OPO₃H₂ and its salt
n=0, 1 or 2 carbon atoms.

In embodiments of Formula (I), R₁, R₂, R₃, R₄ and R₅ are H, A is a bond, X is C, n is 1, and Y₁ and Y₂ are H.

In embodiments of Formula (I), R₁, R₂, and R₃ are an alkyl. In embodiments, the alkyl is a substituted alkyl. In embodiments, alkyl is substituted with a heteroatom or halogen.

In embodiments of Formula (I), R₁, R₂, and R₃ are an cycloalkyl. In embodiments, the cycloalkyl is a substituted cycloalkyl. In embodiments, the cycloalkyl is substituted with a heteroatom or halogen.

In embodiments of Formula (I), R₄ or R₅ is a substituted alkyl. In embodiments, alkyl is substituted with a heteroatom or halogen.

In embodiments of Formula (I), the pro-drugs are selected from aliphatic ester, aromatic ester, heteroaromatic ester, aminal, hemiaminal, CH₂—OPO₃H₂, and —CH-alkyl-OPO₃H₂.

In embodiments, the present disclosure provides compounds of formula (I-A):

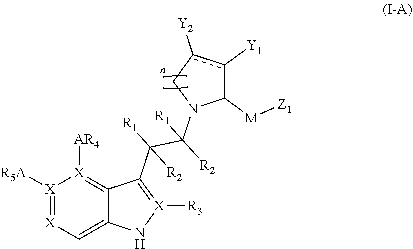

or a pharmaceutically acceptable salt thereof or deuterated form thereof; wherein,
Y₁ and Y₂ are independently hydrogen/deuterium, —CH₂—, —OR₁, —CO₂, or —NR₁,R₂
Z₁ is independently —OH/OD, —SH/SD, OR₁ or —NR₁,R₂
A is H, D, O, N or S
X are independently —C, —N
M is CH₂, C═O, or CR₁R₂
R₁, R₂ and R₃ are independently H or D or alkyl, cycloalkyl, heteroatom substituted, halogen, and halogen substituted alkyl or cycloalkyl, aromatic or heteroaromatic
R₄ and R₅ are independently H or D or alkyl, cycloalkyl, heteroatom substituted, halogen, halogen substituted alkyl or cycloalkyl, aromatic or heteroaromatic
or
R₄ and R₅ are prodrugs, like aliphatic, aromatic, heteroaromatic esters, aminal and hemiaminal, —OPO₃H₂, —CH₂—OPO₃H₂ or —CH alkyl-OPO₃H₂ and its salt
n=0, 1 or 2 carbon atoms.

In embodiments, the present disclosure relates to a compound of Formula (I-A), wherein R₁, R₂, R₃, R₄ and R₅ are H, A is a bond, X is C, n is 1, and Y₁ and Y₂ are H.

In embodiments, the present disclosure provides compounds of Formula (II):

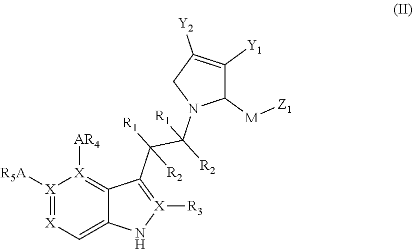

or a pharmaceutically acceptable salt thereof; wherein,
Y₁ and Y₂ are independently hydrogen/deuterium, —OR₁, —CO₂, or —NR₁,R₂
Z₁ is independently —OH/OD, —SH/SD, OR₁, or —NR₁,R₂
A is H, D, O, N or S
X are independently —C, —N
M is CH₂, C═O, or CR₁R₂
R₁, R₂ and R₃ is independently H or D or alkyl, cycloalkyl, heteroatom substituted, and halogen substituted alkyl or cycloalkyl, aromatic or heteroaromatic
R₄ and R₅ are independently H or D or alkyl, cycloalkyl, heteroatom substituted, and halogen substituted alkyl or cycloalkyl, aromatic or heteroaromatic
or
R₄ and R₅ could be prodrugs, like aliphatic, aromatic, heteroaromatic esters, aminal and hemiaminal, —CH₂—OPO₃H₂ or —CH alkyl-OPO₃H₂ and its salt.

In embodiments of Formula (II), R₁, R₂, R₃, R₄ and R₅ are H, A is a bond, X is C, and Y₁ and Y₂ are H.

In embodiments of Formula (II), R₁, R₂, R₃, R₄ and R₅ are H, A is a bond, X is C, n is 1, and Y₁ and Y₂ are H.

In embodiments of Formula (II), R₁, R₂, and R₃ are an alkyl. In embodiments, the alkyl is a substituted alkyl. In embodiments, alkyl is substituted with a heteroatom or halogen.

In embodiments of Formula (II), R₁, R₂, and R₃ are an cycloalkyl. In embodiments, the cycloalkyl is a substituted cycloalkyl. In embodiments, the cycloalkyl is substituted with a heteroatom or halogen.

In embodiments of Formula (II), R₄ or R₅ is a substituted alkyl. In embodiments, alkyl is substituted with a heteroatom or halogen.

In embodiments of Formula (II), the pro-drugs are selected from aliphatic ester, aromatic ester, heteroaromatic ester, aminal, hemiaminal, CH₂—OPO₃H₂, and —CH-alkyl-OPO₃H₂.

In embodiments, the present disclosure provides compounds of Formula (III):

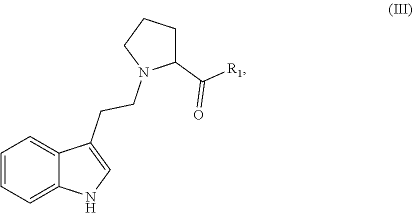

wherein R₁ is O-alkyl, O-cycloalkyl, or hydroxy.

In embodiments of Formula (III), the alkyl is ethyl or methyl.

In embodiments, the present disclosure provides compounds of Formula (II-A):

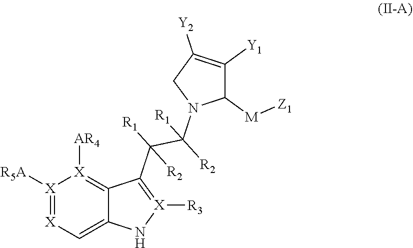

or a pharmaceutically acceptable salt thereof or deuterated form thereof; wherein,
Y₁ and Y₂ are independently hydrogen/deuterium, —OR₁, —CO₂, or —NR₁,R₂
Z₁ is independently —OH/OD, —SH/SD, OR₁, or —NR₁,R₂
A is H, D, O, N or S
X are independently —C, —N
M is CH₂, C═O, or CR₁R₂
R₁, R₂ and R₃ is independently H or D or alkyl, cycloalkyl, heteroatom substituted, halogen, and halogen substituted alkyl or cycloalkyl, aromatic or heteroaromatic
R₄ and R₅ are independently H or D or alkyl, cycloalkyl, heteroatom substituted, halogen, halogen substituted alkyl or cycloalkyl, aromatic or heteroaromatic
or
R₄ and R₅ could be prodrugs, like aliphatic, aromatic, heteroaromatic esters, aminal and hemiaminal, —OPO₃H₂, —CH₂—OPO₃H₂ or —CH alkyl-OPO₃H₂ and its salt.

In embodiments, the present disclosure relates to compounds of Formula (I-B), wherein R₁, R₂, R₃, R₄ and R₅ are H, A is a bond, X is C, and Y₁ and Y₂ are H.

In embodiments, the present disclosure provides a compound of Formula (IV):

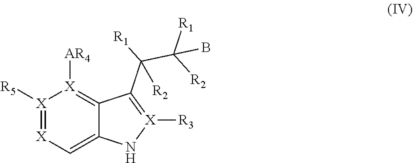

or a pharmaceutically acceptable salt thereof;
wherein,
   A is a bond, H, D, O, N, or S;
   B is a nitrogen-linked C₅-C₇ heterobicycle;
each X is independently —CH, —C, or —N;
R₁, R₂, and R₃ are each independently H, D, alkyl, cycloalkyl, aromatic, or heteroaromatic, wherein R₃ is optionally absent; and
R₄ and R₅ are each independently H, D or alkyl, cycloalkyl, aromatic, or heteroaromatic;
or R₄ and R₅ are prodrugs,
wherein each alkyl, cycloalkyl, aromatic, heteroaromatic and heterobicycle is optionally substituted.

In embodiments of Formula (IV), B is a substituted C₅ heterobicycle.

In embodiments of Formula (IV), B is:

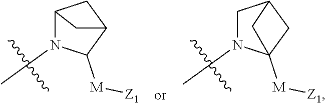

wherein M is absent, —CH₂—, —C═O, or —CR₁R₂; and
Z₁ is —OH, —OD, —SH, —SD, —NR₁R₂, O-alkyl, O-cycloalkyl, or O-aromatic,
wherein when M is absent, Z₁ is CN.

In embodiments of Formula (IV), each X is —CH.

In embodiments, the present disclosure provides a compound of Formula (V):

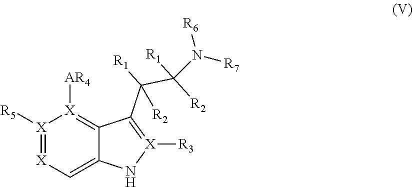

or a pharmaceutically acceptable salt thereof;
wherein,
   A is a bond, H, D, O, N, or S;
each X is independently —CH, —C, or —N;
each R₁, R₂, and R₃ are each independently H, D, alkyl, cycloalkyl, aromatic, or heteroaromatic, wherein R₃ is optionally absent; and
R₄ and R₅ are each independently H, D or alkyl, cycloalkyl, aromatic, or heteroaromatic;
or R₄ and R₅ are prodrugs,
R₆ is C₁-C₃ alkyl;
R₇ is -(alkyl)COOH, -(alkyl)COOR₁, and C₁-C₃ alkyl, wherein each alkyl, cycloalkyl, aromatic and heteroaromatic is optionally substituted.

In embodiments of Formula (V), each X is —CH.

In embodiments of Formula (V), R₆ is methyl.

In embodiments, the present disclosure provides a compound of Formula (I-B),

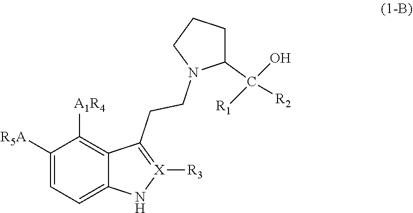

or a pharmaceutically acceptable salt or deuterated form thereof;
wherein,
R₁ and R₂ are each independently H or alkyl;
R₃ is H, D, alkyl, cycloalkyl, aromatic, halogen, heteroaromatic, or absent;
A₁ and A₂ are each independently a bond, H, D, O, N, or S;
R₃ is H, D, alkyl, cycloalkyl, aromatic, halogen, or heteroaromatic, wherein R₃ is optionally absent;
R₄ and R₅ are each independently H, D, alkyl, cycloalkyl, aromatic, halogen substituted alkyl or cycloalkyl, halogen, or heteroaromatic, or
R₄ and R₅ are each independently a prodrug; and
X is independently —CH, —C, or —N.

In embodiments of Formula (I-B), R₁ is H.

In embodiments of Formula (I-B), R₁ is alkyl.

In embodiments of Formula (I-B), R₂ is H.

In embodiments of Formula (I-B), R₂ is alkyl.

In embodiments of Formula (I-B), A₁ is a bond.

In embodiments of Formula (I-B), A₁ is O.

In embodiments of Formula (I-B), A₂ is a bond.

In embodiments of Formula (I-B), A₂ is O.

In embodiments of Formula (I-B), R₃ absent.

In embodiments of Formula (I-B), R₃ is halogen.

In embodiments of Formula (I-B), R₃ is F, Cl, or Br.

In embodiments of Formula (I-B), R₄ is H.

In embodiments of Formula (I-B), R₄ is an alkyl. In embodiments, alkyl is —CH₃.

In embodiments of Formula (I-B), R₄ is a halogen substituted alkyl. In embodiments, the halogen substituted alkyl is —CF₃.

In embodiments of Formula (I-B), R₄ is a halogen.

In embodiments of Formula (I-B), R₄ is F, Cl, or Br.

In embodiments of Formula (I-B), R₄ is a prodrug. In embodiments, the prodrug is an aliphatic ester, aromatic ester, heteroaromatic ester, aminal, hemiaminal, —OPO₃H₂, —CH₂—OPO₃H₂, and —CH-alkyl-OPO₃H₂.

In embodiments of Formula (I-B), R₅ is H.

In embodiments of Formula (I-B), R₅ is an alkyl. In embodiments, the alkyl is —CH₃.

In embodiments of Formula (I-B), R₅ is a halogen substituted alkyl. In embodiments, the halogen substituted alkyl is —CF₃.

In embodiments of Formula (I-B), R₅ is a halogen.

In embodiments of Formula (I-B), R₅ is F, Cl, or Br.

In embodiments of Formula (I-B), R₅ is a prodrug. In embodiments, the prodrug is an aliphatic ester, aromatic ester, heteroaromatic ester, aminal, hemiaminal, —OPO₃H₂, —CH₂—OPO₃H₂, and —CH-alkyl-OPO₃H₂.

In embodiments of Formula (I-B), at least one of R₄ or R₅ is a prodrug.

In embodiments of Formula (I-B), X is CH. In embodiments, X is CH and R₃ is absent.

In embodiments of Formula (I-B), X is —C—. In embodiments, X is —C— and R₃ is halogen.

In embodiments of Formula (I-B), A₁ is a bond and R₄ is H.

In embodiments of Formula (I-B), A₁ is a bond and R₄ is a halogen.

In embodiments of Formula (I-B), A₁ is a O and R₄ is an alkyl. In embodiments, the alkyl is —CH₃.

In embodiments of Formula (I-B), A₁ is O and R₄ is a halogen substituted alkyl. In embodiments, the halogen substituted alkyl is —CF₃.

In embodiments of Formula (I-B), A₁ is a bond and R₅ is H.

In embodiments of Formula (I-B), A₁ is a bond and R₅ is a halogen.

In embodiments of Formula (I-B), A₁ is a O and R₅ is an alkyl. In embodiments, the alkyl is —CH₃.

In embodiments of Formula (I-B), A₁ is O and R₅ is a halogen substituted alkyl. In embodiments, the alkyl is —CF₃.

In embodiments, the present disclosure provides a compound of Formula (I-C),

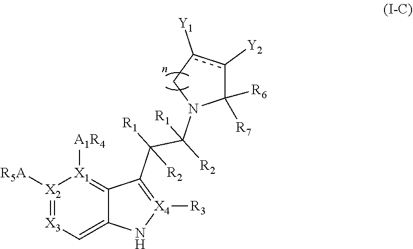

or a pharmaceutically acceptable salt or deuterated form thereof;
wherein,
Y₁ and Y₂ are each independently hydrogen (H), deuterium (D), —CH₃, —OR₁, —CO₂R₈, —NR₁₀R₁₁;
X₁, X₂, X₃, or X₄ are each independently —CH, —C, or —N;
A₁ and A₂ are each independently a bond, H, D, O, N, or S;
R₁, R₂, and R₃ are each independently H, D, alkyl, cycloalkyl, aromatic, halogen, heteroaromatic, or absent;
R₄ and R₅ are each independently H, D, alkyl, cycloalkyl, aromatic, halogen substituted alkyl or cycloalkyl, halogen, heteroaromatic, —C(O)CH₃, or —P(O)(OR₉)₂;
R₆ is H or alkyl;
R₇ is

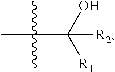

—CO₂R₈, —CH₂C(O)CH₃, —CH₂CO₂R₈, —OP(O)(OR₉)₂, —C(O)—NR₁₀R₁₁;
R₈ is H, alkyl, cycloalkyl, haloalkyl, alkyl-O-alkyl, or aryl;
R₉ is H or alkyl;
R₁₀ and R₁₁ are each independently H or alkyl; and
n is 0, 1, or 2, wherein the compound is not

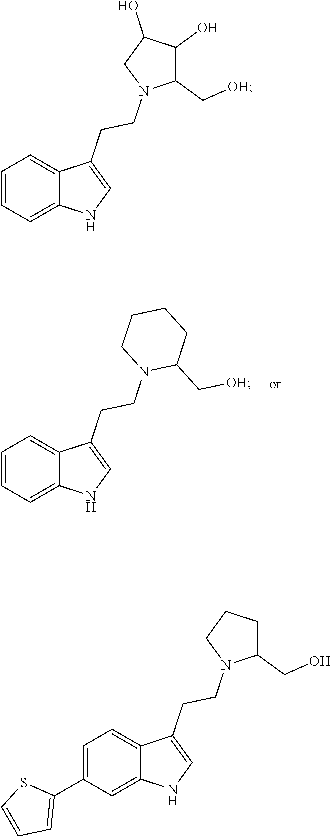 [SMILES: OCC1C(O)C(O)CN1CCc1c[nH]c2ccccc12 ; OCC1CCCCN1CCc1c[nH]c2ccccc12 ; OCC1CCCN1CCc1c[nH]c2cc(-c3cccs3)ccc12]

and wherein when n is 2, R⁷ is not —CO₂R₈ or —CH₂CO₂R₈.

In embodiments of Formula (I-C), Y₁ and Y₂ are each H.

In embodiments of Formula (I-C), X₁ is —C.

In embodiments of Formula (I-C), X₁ is N.

In embodiments of Formula (I-C), X₂ is —C.

In embodiments of Formula (I-C), X₃ is N.

In embodiments of Formula (I-C), X₄ is —C.

In embodiments of Formula (I-C), A₁ is O.

In embodiments of Formula (I-C), A₂ is O.

In embodiments of Formula (I-C), R₁ is H.

In embodiments of Formula (I-C), R₂ is H.

In embodiments of Formula (I-C), R₃ is H.

In embodiments of Formula (I-C), R₃ is halogen.

In embodiments of Formula (I-C), R₄ is H.

In embodiments of Formula (I-C), R₅ is H.

In embodiments of Formula (I-C), A₁ is O and R₄ is H.

In embodiments of Formula (I-C), A₂ is O and R₅ is H.

In embodiments of Formula (I-C), A₁ is O and R₄ is —P(O)(OR₉)₂.

In embodiments of Formula (I-C), A₂ is O and R₅ is —P(O)(OR₉)₂.

In embodiments of Formula (I-C), A₁ is a bond and R₅ is halogen.

In embodiments of Formula (I-C), A₂ is a bond and R₄ is halogen.

In embodiments of Formula (I-C), A₁ is O and R₄ is H.

In embodiments of Formula (I-C), A₂ is O and R₅ is H.

In embodiments of Formula (I-C), A₁ is O and R₄ is alkyl.

In embodiments of Formula (I-C), A₂ is O and R₅ is alkyl.

In embodiments of Formula (I-C), A₁ is O and R₄ is —C(O)CH₃.

In embodiments of Formula (I-C), A₂ is O and R₅ is —C(O)CH₃.

In embodiments of Formula (I-C), R₆ is H.

In embodiments of Formula (I-C), R₆ is alkyl. In embodiments, the alkyl is a methyl.

In embodiments of Formula (I-C), R₇ is

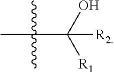

In embodiments, R₁ and R₂ are each H. In embodiments, at least one of R₁ or R₂ is alkyl. In embodiments, one of R₁ or R₂ is alkyl and one of R₁ or R₂ is H.

In embodiments of Formula (I-C), R₁ is —CO₂R₈. In embodiments, R₈ is H. In embodiments, R₈ is alkyl.

In embodiments of Formula (I-C), R₇ is —CH₂C(O)CH₃.

In embodiments of Formula (I-C), R₁ is —CH₂CO₂R₈. In embodiments, R₈ is H. In embodiments, R₈ is alkyl.

In embodiments of Formula (I-C), R₁ is —OP(O)(OR₉)₂. In embodiments, R₉ is H. In embodiments, R₉ is alkyl.

In embodiments of Formula (I-C), R₁ is —C(O)—NR₁₀R₁₁. In embodiments, R₁₀ and R₁₁ are each H. In embodiments, at least one of R₁₀ or R₁₁ are alkyl. In embodiments one of R₁₀ or R₁₁ is alkyl and one of R₁₀ or R₁₁ is H.

In embodiments of Formula (I-C), n is 0.

In embodiments of Formula (I-C), n is 1.

In embodiments of Formula (I-C), n is 2.

In embodiments of the present disclosure, the compound of Formula (I), (I-A), (I-B), (I-C), (II), (II-A), (III), and (IV), is a compound of Table 1.

TABLE 1
Compounds of the invention

| | | | | | | 5-HT2A, | 5-HT2B, |
|---|---|---|---|---|---|---|---|
| | | | | | | IPOne | IPOne |
| | | 5-HT2A, | | | 5-HT2B, | EC50 | EC50 |
| | | Ca++ flux | | 5-HT2C, | Ca++ flux | (uM)/% | (uM)/% |
| | | EC50 | 5-HT2A, | Ca++ | EC50 | activation | activation |
| | | (uM)/% | b-arrestin | flux | (uM)/% | @30 uM/% | @30 uM/% |
| | | activation | EC50 | EC50 | activation | activation | activation |
| # | Structure | @30 uM | (uM) | (uM) | @30 uM | @0.3 uM | @0.3 uM |
| 1 | 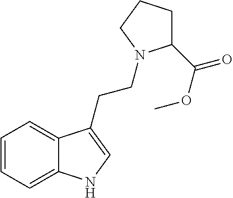 [SMILES: COC(=O)C1CCCN1CCc1c[nH]c2ccccc12] | 0.987/78.24 | 16.132 | 4.162 | >30/11.15 | — | — |
| 2 | 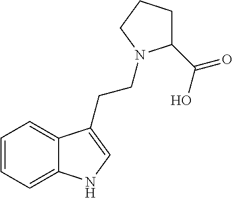 [SMILES: O=C(O)C1CCCN1CCc1c[nH]c2ccccc12] | >30/2.41 | — | — | — | — | — |
| 3 | 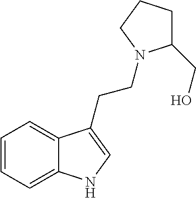 [SMILES: OCC1CCCN1CCc1c[nH]c2ccccc12] | 0.103/94.73 | 1.154 | 0.498 | >30/23.51 | —/97.47/47.92 | —/93.91/25.92 |
| 4 | 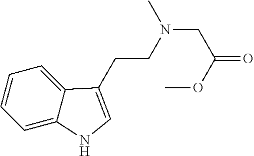 [SMILES: COC(=O)CN(C)CCc1c[nH]c2ccccc12] | 0.302/36.92 | — | — | — | — | — |
| 5 | 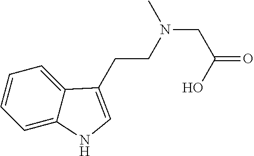 [SMILES: CN(CCc1c[nH]c2ccccc12)CC(=O)O] | >30/24.51 | — | — | — | — | — |
| 6 | 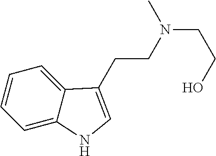 [SMILES: CN(CCO)CCc1c[nH]c2ccccc12] | 0.101/42.83 | — | — | — | — | — |
| 7 | 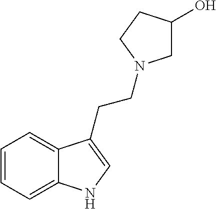 [SMILES: OC1CCN(CCc2c[nH]c3ccccc23)C1] | — | — | — | — | —/42.05/7.01 | —/28.66/5.71 |
| 8 | 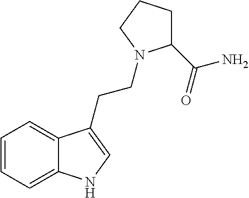 [SMILES: NC(=O)C1CCCN1CCc1c[nH]c2ccccc12] | — | — | — | — | — | — |
| 9 | 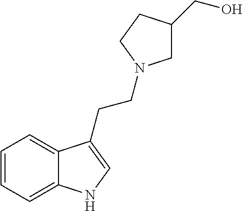 [SMILES: OCC1CCN(CCc2c[nH]c3ccccc23)C1] | — | — | — | — | — | — |
| 10 | 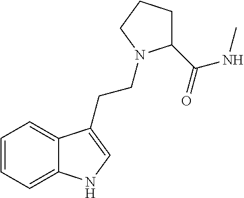 [SMILES: CNC(=O)C1CCCN1CCc1c[nH]c2ccccc12] | — | — | — | — | — | — |
| 11 | 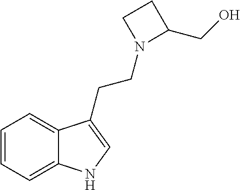 [SMILES: OCC1CCN1CCc1c[nH]c2ccccc12] | — | — | — | — | — | — |
| 12 | 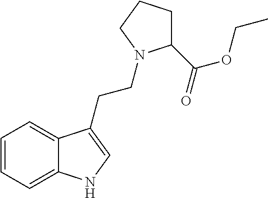 [SMILES: CCOC(=O)C1CCCN1CCc1c[nH]c2ccccc12] | — | — | — | — | — | — |
| 13 | 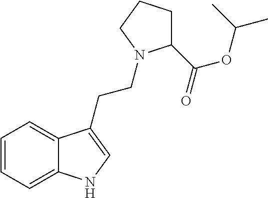 [SMILES: CC(C)OC(=O)C1CCCN1CCc1c[nH]c2ccccc12] | — | — | — | — | — | — |
| 14 | 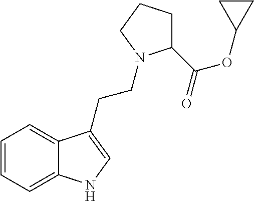 [SMILES: O=C(OC1CC1)C1CCCN1CCc1c[nH]c2ccccc12] | — | — | — | — | — | — |
| 15 | 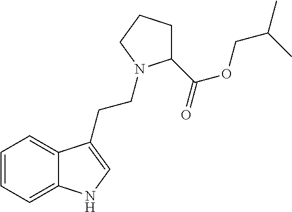 [SMILES: CC(C)COC(=O)C1CCCN1CCc1c[nH]c2ccccc12] | — | — | — | — | — | — |
| 16 | 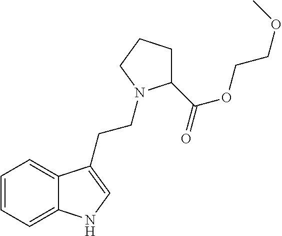 [SMILES: COCCOC(=O)C1CCCN1CCc1c[nH]c2ccccc12] | — | — | — | — | — | — |
| 17 | 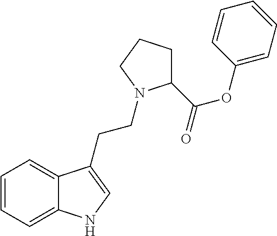 [SMILES: O=C(Oc1ccccc1)C1CCCN1CCc1c[nH]c2ccccc12] | — | — | — | — | — | — |
| 18 | 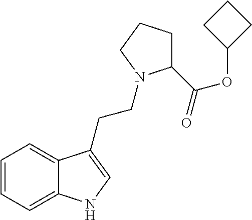 [SMILES: O=C(OC1CCC1)C1CCCN1CCc1c[nH]c2ccccc12] | — | — | — | — | — | — |
| 19 | 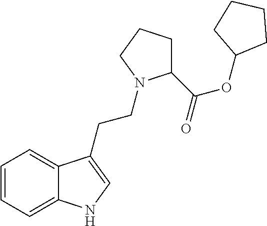 [SMILES: O=C(OC1CCCC1)C1CCCN1CCc1c[nH]c2ccccc12] | — | — | — | — | — | — |
| 20 | 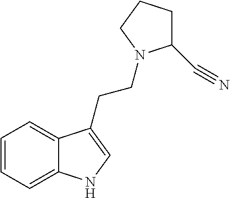 [SMILES: N#CC1CCCN1CCc1c[nH]c2ccccc12] | — | — | — | — | — | — |
| 21 | 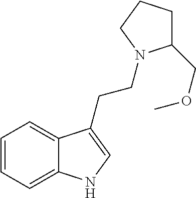 [SMILES: COCC1CCCN1CCc1c[nH]c2ccccc12] | — | — | — | — | — | — |
| 22 | 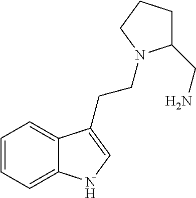 [SMILES: NCC1CCCN1CCc1c[nH]c2ccccc12] | — | — | — | — | —/99.0/10.22 | —/99.61/20.0 |
| 23 | 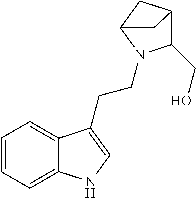 [SMILES: OCC1C2CC(C2)N1CCc1c[nH]c2ccccc12] | — | — | — | — | — | — |
| 24 | 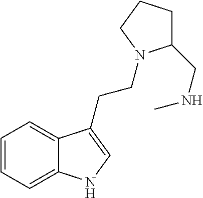 [SMILES: CNCC1CCCN1CCc1c[nH]c2ccccc12] | — | — | — | — | — | — |
| 25 | 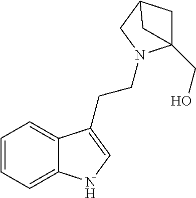 [SMILES: OCC12CC(CN1CCc1c[nH]c3ccccc13)C2] | — | — | — | — | — | — |
| 26 | 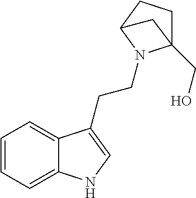 [SMILES: OCC12CCC(C1)N2CCc1c[nH]c2ccccc12] | — | — | — | — | — | — |
| 27 | 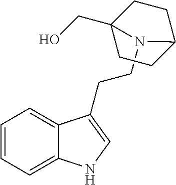 [SMILES: OCC12CCC(CC1)N2CCc1c[nH]c2ccccc12] | — | — | — | — | — | — |
| 28 | 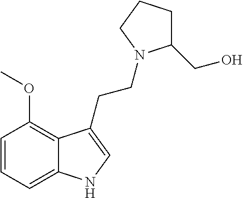 [SMILES: COc1cccc2[nH]cc(CCN3CCCC3CO)c12] | | — | | | — | — |
| 29 | 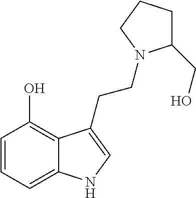 [SMILES: OCC1CCCN1CCc1c[nH]c2cccc(O)c12] | — | — | — | — | —/99.17/99.36 | —/96.36/90.84 |
| 30 | 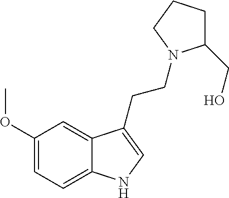 [SMILES: COc1ccc2[nH]cc(CCN3CCCC3CO)c2c1] | — | — | — | — | —/100.01/ 97.84 | —/98.82/89.41 |
| 31 | 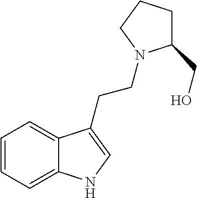 [SMILES: OC[C@@H]1CCCN1CCc1c[nH]c2ccccc12] | — | — | — | — | 0.0621/99.55/ 76.49 | 0.126/97.03/ 54.63 |
| 32 | 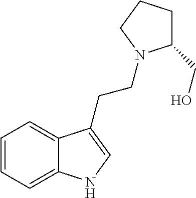 [SMILES: OC[C@H]1CCCN1CCc1c[nH]c2ccccc12] | — | — | — | — | —/42.07/7.16 | —/33.88/4.28 |
| 33 | 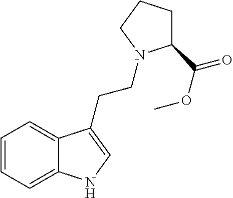 [SMILES: COC(=O)[C@@H]1CCCN1CCc1c[nH]c2ccccc12] | — | — | — | — | 0.806/94.38/ 44.0 | 0.456/91.27/ 33.94 |
| 34 | 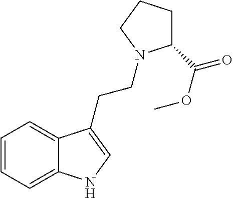 [SMILES: COC(=O)[C@H]1CCCN1CCc1c[nH]c2ccccc12] | — | — | — | — | —/29.89/4.60 | —/35.10/1.34 |
| 35 | 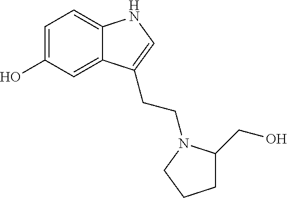 [SMILES: OCC1CCCN1CCc1c[nH]c2ccc(O)cc12] | — | — | — | — | —/99.46/96.96 | —/98.69/93.73 |
| 36 | 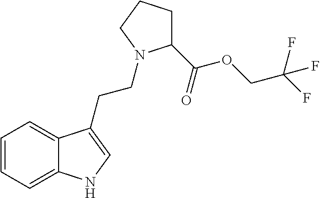 [SMILES: O=C(OCC(F)(F)F)C1CCCN1CCc1c[nH]c2ccccc12] | — | — | — | — | — | — |
| 37 | 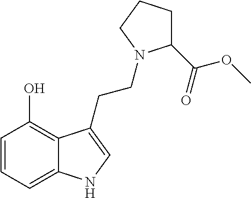 [SMILES: COC(=O)C1CCCN1CCc1c[nH]c2cccc(O)c12] | — | — | — | — | — | — |
| 38 | 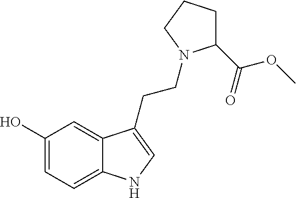 [SMILES: COC(=O)C1CCCN1CCc1c[nH]c2ccc(O)cc12] | — | — | — | — | — | — |
| 39 | 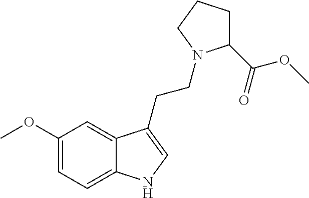 | — | — | — | — | — | — |
| 40 | 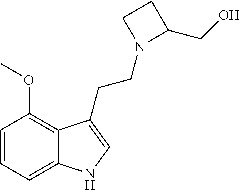 [SMILES: COc1cccc2[nH]cc(CCN3CCC3CO)c12] | — | — | — | — | — | — |
| 41 | 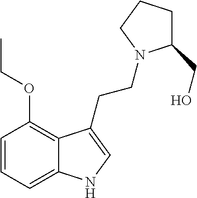 [SMILES: CCOc1cccc2[nH]cc(CCN3CCC[C@H]3CO)c12] | — | — | — | — | — | — |
| 42 | 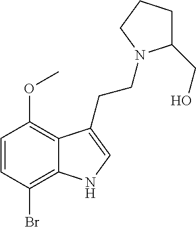 [SMILES: COc1ccc(Br)c2[nH]cc(CCN3CCCC3CO)c12] | — | — | — | — | —/94.07/78.52 | —/46.21/22.01 |
| 43 | 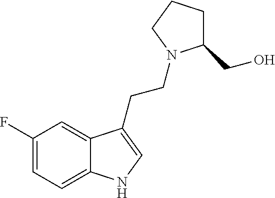 [SMILES: OC[C@@H]1CCCN1CCc1c[nH]c2ccc(F)cc12] | — | — | — | — | 0.0077/89.41/ 86.28 | 0.0089/85.65/ 79.75 |
| 44 | 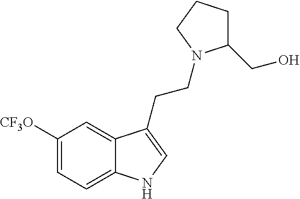 | — | — | — | — | — | — |
| 45 | 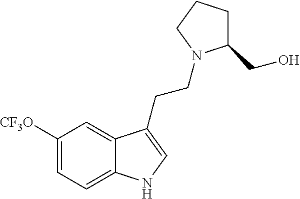 | — | — | — | — | — | — |
| 46 | 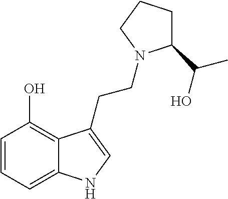 [SMILES: CC(O)[C@@H]1CCCN1CCc1c[nH]c2cccc(O)c12] | — | — | — | — | — | — |
| 47 | 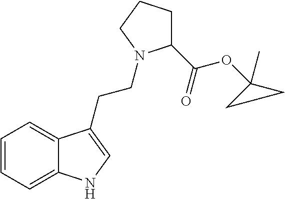 [SMILES: CC1(OC(=O)C2CCCN2CCc2c[nH]c3ccccc23)CC1] | | — | — | | | |
| 48 | 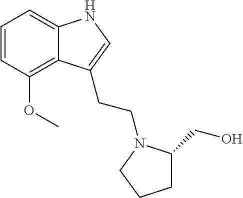 [SMILES: COc1cccc2[nH]cc(CCN3CCC[C@H]3CO)c12] | — | — | — | — | 0.0112/90.92/ 83.18 | 0.0222/83.41/ 62.03 |
| 49 | 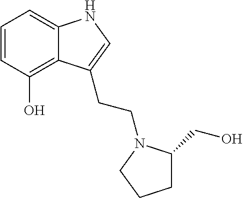 [SMILES: OC[C@@H]1CCCN1CCc1c[nH]c2cccc(O)c12] | — | — | — | — | 0.0225/100.25/ 99.16 | 0.0432/99.07/ 91.95 |
| 50 | 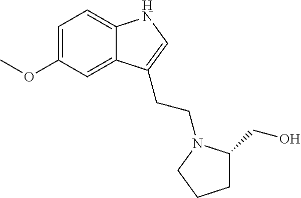 [SMILES: COc1ccc2[nH]cc(CCN3CCC[C@H]3CO)c2c1] | — | — | — | — | 0.0125/102.58/ 100.46 | 0.0088/101.27/ 101.15 |
| 51 | 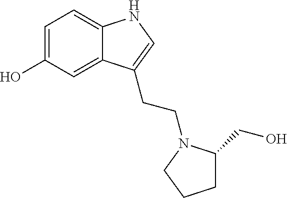 [SMILES: OC[C@@H]1CCCN1CCc1c[nH]c2ccc(O)cc12] | — | — | — | — | 0.0456/101.06/ 98.99 | 0.0867/99.13/ 85.65 |
| 52 | 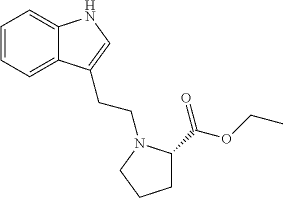 [SMILES: CCOC(=O)[C@@H]1CCCN1CCc1c[nH]c2ccccc12] | — | — | — | — | 0.8881/101.33/ 24.01 | 2.561/99.18/ 25.36 |
| 53 | 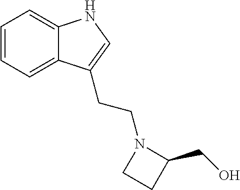 [SMILES: OC[C@H]1CCN1CCc1c[nH]c2ccccc12] | — | — | — | — | — /70.70/3.07 | —/74.66/16.27 |
| 54 | 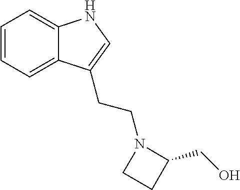 [SMILES: OC[C@@H]1CCN1CCc1c[nH]c2ccccc12] | — | — | — | — | 0.2492/100.54/ 81.12 | 0.78/97.93/ 61.97 |
| 55 | 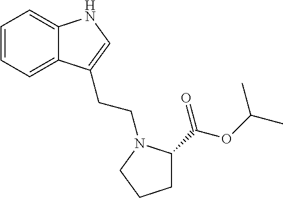 [SMILES: CC(C)OC(=O)[C@@H]1CCCN1CCc1c[nH]c2ccccc12] | — | — | — | — | 0.3582/101.20/ 68.09 | 1.037/102.28/ 37.70 |
| 56 | 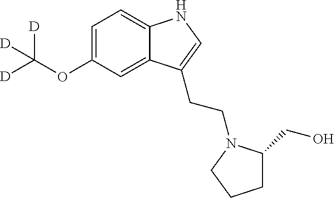 [SMILES: [2H]C([2H])([2H])Oc1ccc2[nH]cc(CCN3CCC[C@H]3CO)c2c1] | — | — | — | — | | |
| 57 | 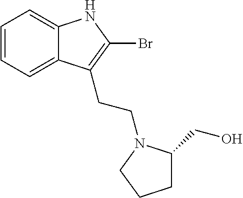 [SMILES: OC[C@@H]1CCCN1CCc1c(Br)[nH]c2ccccc12] | — | — | — | — | —/30.88/4.69 | —/73.02/−1.03 |
| 58 | 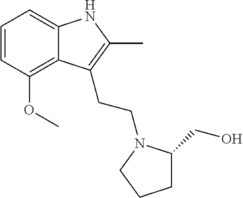 [SMILES: COc1cccc2[nH]c(C)c(CCN3CCC[C@H]3CO)c12] | — | — | — | — | | |
| 59 | 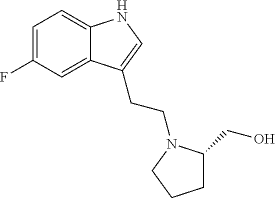 [SMILES: OC[C@@H]1CCCN1CCc1c[nH]c2ccc(F)cc12] | — | — | — | — | 0.0077/89.41/ 86.28 | 0.0089/85.65/ 79.75 |
| 60 | 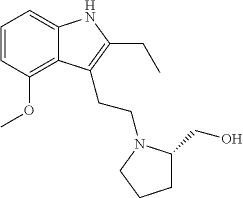 [SMILES: CCc1[nH]c2cccc(OC)c2c1CCN1CCC[C@H]1CO] | — | — | — | — | — | — |
| 61 | 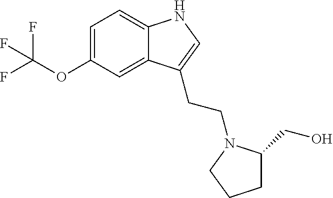 [SMILES: OC[C@@H]1CCCN1CCc1c[nH]c2ccc(OC(F)(F)F)cc12] | — | — | — | — | 0.076/88.30/ 46.15 | 0.0577/76.17/ 37.52 |
| 62 | 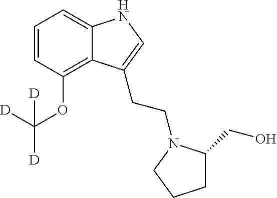 [SMILES: [2H]C([2H])([2H])Oc1cccc2[nH]cc(CCN3CCC[C@H]3CO)c12] | — | — | — | — | — | — |
| 63 | 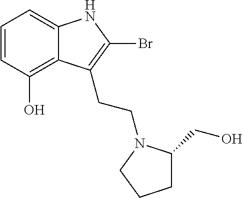 [SMILES: OC[C@@H]1CCCN1CCc1c(Br)[nH]c2cccc(O)c12] | — | — | — | — | — | — |
| 64 | 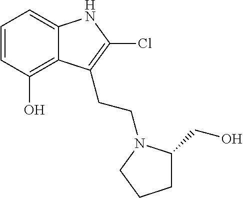 [SMILES: OC[C@@H]1CCCN1CCc1c(Cl)[nH]c2cccc(O)c12] | — | — | — | — | — | — |
| 65 | 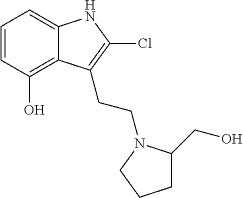 [SMILES: OCC1CCCN1CCc1c(Cl)[nH]c2cccc(O)c12] | — | — | — | — | — | — |
| 66 | 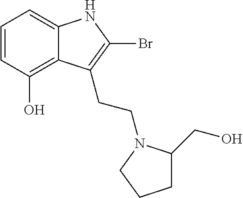 [SMILES: OCC1CCCN1CCc1c(Br)[nH]c2cccc(O)c12] | — | — | — | — | — | — |
| 67 | 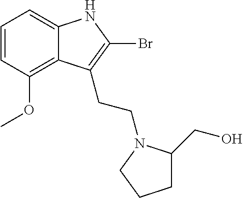 [SMILES: COc1cccc2[nH]c(Br)c(CCN3CCCC3CO)c12] | — | — | — | — | —/95.94/27.24 | —/87.13/19.16 |
| 68 | 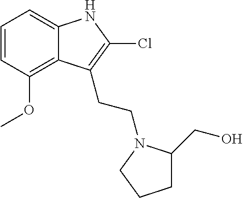 [SMILES: COc1cccc2[nH]c(Cl)c(CCN3CCCC3CO)c12] | — | — | — | — | — | — |
| 69 | 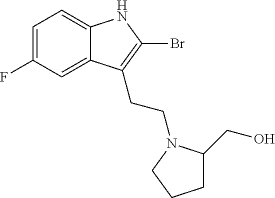 [SMILES: OCC1CCCN1CCc1c(Br)[nH]c2ccc(F)cc12] | — | — | — | — | —/83.80/15.11 | —/90.52/27.62 |
| 70 | 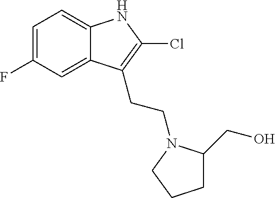 [SMILES: OCC1CCCN1CCc1c(Cl)[nH]c2ccc(F)cc12] | — | — | — | — | —/97.68/61.05 | —/99.82/55.14 |
| 71 | 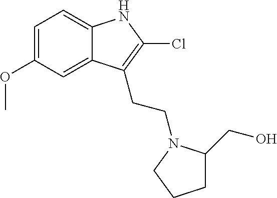 [SMILES: COc1ccc2[nH]c(Cl)c(CCN3CCCC3CO)c2c1] | — | — | — | — | — | — |
| 72 | 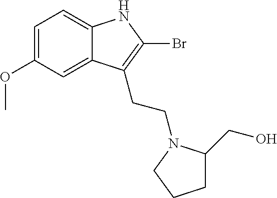 [SMILES: COc1ccc2[nH]c(Br)c(CCN3CCCC3CO)c2c1] | — | — | — | — | —/37.59/5.63 | —/41.69/13.48 |
| 73 | 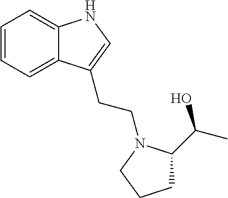 [SMILES: C[C@H](O)[C@@H]1CCCN1CCc1c[nH]c2ccccc12] | — | — | — | — | 0.5567/101.06/ 61.89 | 0.9577/104.22/ 61.22 |
| 74 | 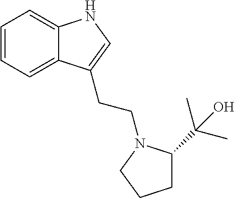 [SMILES: CC(C)(O)[C@@H]1CCCN1CCc1c[nH]c2ccccc12] | — | — | — | — | 0.2467/102.00/ 80.25 | 0.526/100.03/ 63.02 |
| 75 | 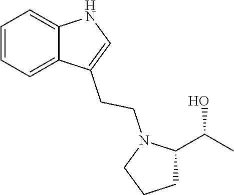 [SMILES: C[C@@H](O)[C@@H]1CCCN1CCc1c[nH]c2ccccc12] | — | — | — | — | —/87.30/23.39 | —/80.25/5.88 |
| 76 | 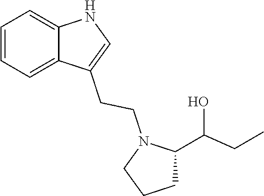 [SMILES: CCC(O)[C@@H]1CCCN1CCc1c[nH]c2ccccc12] | — | — | — | — | — | — |
| 77 | 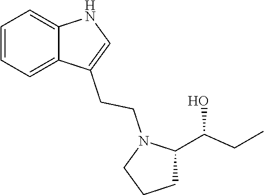 [SMILES: CC[C@@H](O)[C@@H]1CCCN1CCc1c[nH]c2ccccc12] | — | — | — | — | —/90.60/23.91 | —/86.74/18.24 |
| 78 | 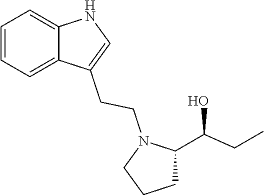 [SMILES: CC[C@H](O)[C@@H]1CCCN1CCc1c[nH]c2ccccc12] | — | — | — | — | —/64.28/1.39 | —/82.22/5.16 |
| 79 | 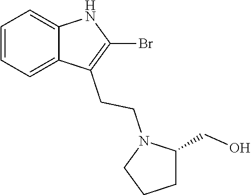 [SMILES: OC[C@@H]1CCCN1CCc1c(Br)[nH]c2ccccc12] | | | | | | |
| 80 | 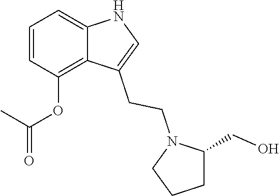 [SMILES: CC(=O)Oc1cccc2[nH]cc(CCN3CCC[C@H]3CO)c12] | — | — | — | — | — | — |
| 81 | 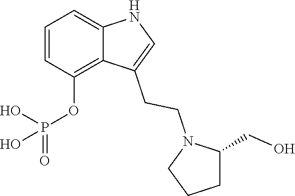 [SMILES: O=P(O)(O)Oc1cccc2[nH]cc(CCN3CCC[C@H]3CO)c12] | — | — | — | — | — | — |
| 82 | 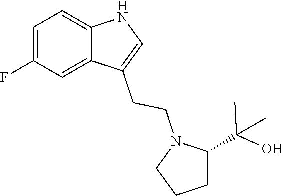 [SMILES: CC(C)(O)[C@@H]1CCCN1CCc1c[nH]c2ccc(F)cc12] | — | — | — | — | — | — |
| 83 | 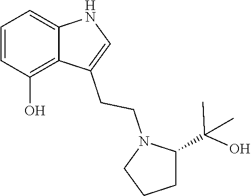 [SMILES: CC(C)(O)[C@@H]1CCCN1CCc1c[nH]c2cccc(O)c12] | — | — | — | — | — | — |
| 84 | 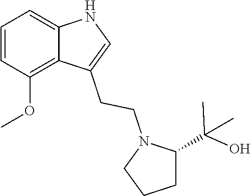 [SMILES: COc1cccc2[nH]cc(CCN3CCC[C@H]3C(C)(C)O)c12] | — | — | — | — | — | — |
| 85 | 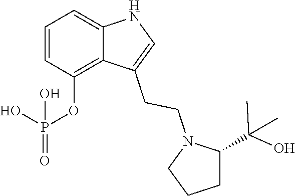 [SMILES: CC(C)(O)[C@@H]1CCCN1CCc1c[nH]c2cccc(OP(=O)(O)O)c12] | — | — | — | — | — | — |
| 86 | 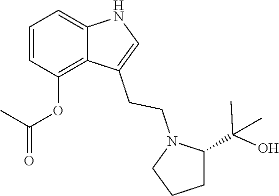 [SMILES: CC(=O)Oc1cccc2[nH]cc(CCN3CCC[C@H]3C(C)(C)O)c12] | — | — | — | — | — | — |
| 87 | 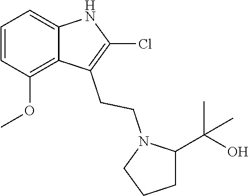 [SMILES: COc1cccc2[nH]c(Cl)c(CCN3CCCC3C(C)(C)O)c12] | — | — | — | — | — | — |
| 88 | 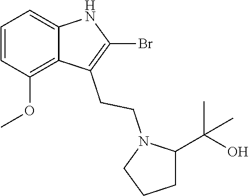 [SMILES: COc1cccc2[nH]c(Br)c(CCN3CCCC3C(C)(C)O)c12] | — | — | — | — | — | — |
| 89 | 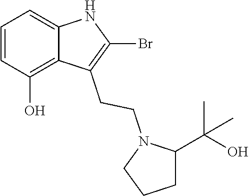 [SMILES: CC(C)(O)C1CCCN1CCc1c(Br)[nH]c2cccc(O)c12] | — | — | — | — | — | — |
| 90 | 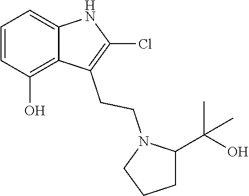 [SMILES: CC(C)(O)C1CCCN1CCc1c(Cl)[nH]c2cccc(O)c12] | — | — | — | — | — | — |
| 91 | 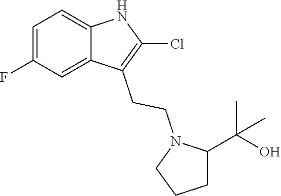 [SMILES: CC(C)(O)C1CCCN1CCc1c(Cl)[nH]c2ccc(F)cc12] | — | — | — | — | — | — |
| 92 | 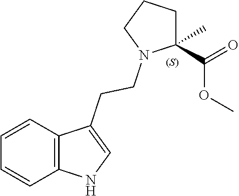 | — | — | — | — | —/−6.28/ −16.26 | —/15.37/ −2.23 |
| 93 | 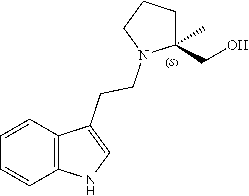 | — | — | — | — | —/1.82/ −11.26 | —/25.58/ −0.54 |
| 94 | 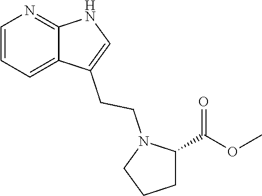 [SMILES: COC(=O)[C@@H]1CCCN1CCc1c[nH]c2ncccc12] | — | — | — | — | —/54.29/0.14 | —/2.48/ −0.22 |
| 95 | 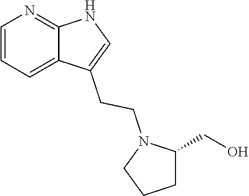 [SMILES: OC[C@@H]1CCCN1CCc1c[nH]c2ncccc12] | — | — | — | — | —/84.32/0.81 | —/9.66/1.09 |
| 96 | 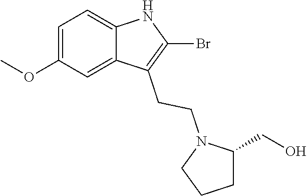 [SMILES: COc1ccc2[nH]c(Br)c(CCN3CCC[C@H]3CO)c2c1] | | | | | | |
| 97 | 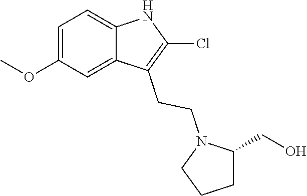 [SMILES: COc1ccc2[nH]c(Cl)c(CCN3CCC[C@H]3CO)c2c1] | | | | | | |
| 98 | 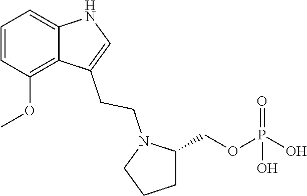 [SMILES: COc1cccc2[nH]cc(CCN3CCC[C@H]3COP(=O)(O)O)c12] | | | | | | |
| 99 | 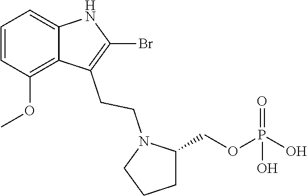 [SMILES: COc1cccc2[nH]c(Br)c(CCN3CCC[C@H]3COP(=O)(O)O)c12] | | | | | | |
| 100 | 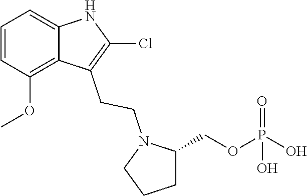 [SMILES: COc1cccc2[nH]c(Cl)c(CCN3CCC[C@H]3COP(=O)(O)O)c12] | | | | | | |
| 101 | 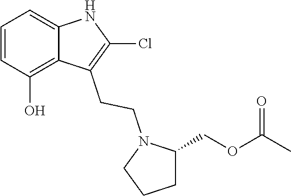 [SMILES: CC(=O)OC[C@@H]1CCCN1CCc1c(Cl)[nH]c2cccc(O)c12] | | | | | | |
| 102 | 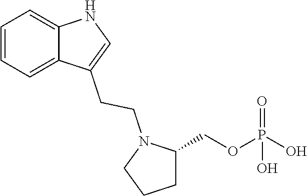 [SMILES: O=P(O)(O)OC[C@@H]1CCCN1CCc1c[nH]c2ccccc12] | | | | | | |
| 103 | 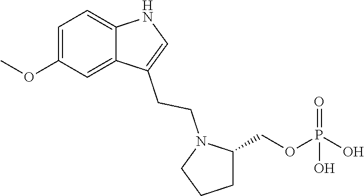 [SMILES: COc1ccc2[nH]cc(CCN3CCC[C@H]3COP(=O)(O)O)c2c1] | | | | | | |
| 104 | 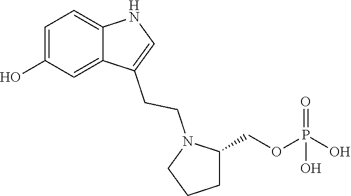 [SMILES: O=P(O)(O)OC[C@@H]1CCCN1CCc1c[nH]c2ccc(O)cc12] | | | | | | |
| 105 | 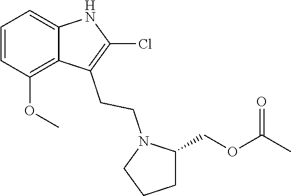 [SMILES: COc1cccc2[nH]c(Cl)c(CCN3CCC[C@H]3COC(C)=O)c12] | | | | | | |
| 106 | 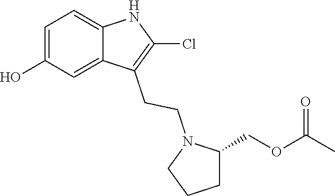 [SMILES: CC(=O)OC[C@@H]1CCCN1CCc1c(Cl)[nH]c2ccc(O)cc12] | | | | | | |
| 107 | 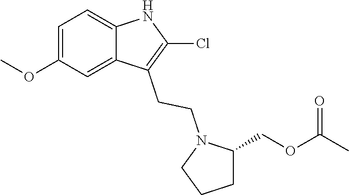 [SMILES: COc1ccc2[nH]c(Cl)c(CCN3CCC[C@H]3COC(C)=O)c2c1] | | | | | | |
| 108 | 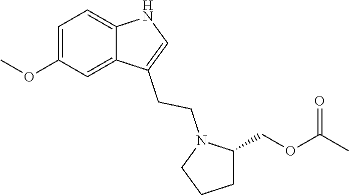 [SMILES: COc1ccc2[nH]cc(CCN3CCC[C@H]3COC(C)=O)c2c1] | | | | | | |
| 109 | 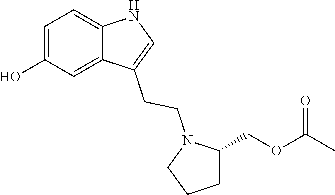 [SMILES: CC(=O)OC[C@@H]1CCCN1CCc1c[nH]c2ccc(O)cc12] | | | | | | |
| 110 | 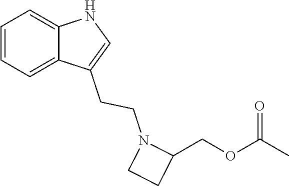 [SMILES: CC(=O)OCC1CCN1CCc1c[nH]c2ccccc12] | | | | | | |
| 111 | 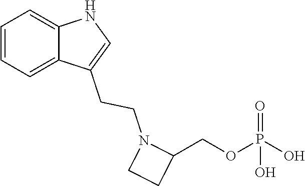 [SMILES: O=P(O)(O)OCC1CCN1CCc1c[nH]c2ccccc12] | | | | | | |
| 112 | 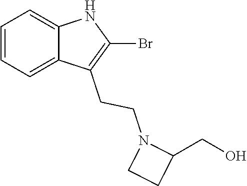 [SMILES: OCC1CCN1CCc1c(Br)[nH]c2ccccc12] | | | | | | |
| 113 | 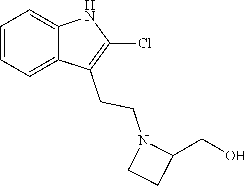 [SMILES: OCC1CCN1CCc1c(Cl)[nH]c2ccccc12] | | | | | | |
| 114 | 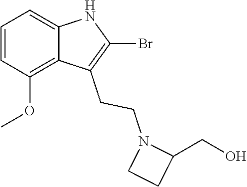 [SMILES: COc1cccc2[nH]c(Br)c(CCN3CCC3CO)c12] | | | | | | |
| 115 | 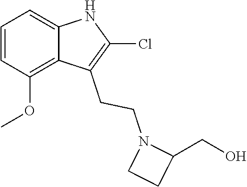 [SMILES: COc1cccc2[nH]c(Cl)c(CCN3CCC3CO)c12] | | | | | | |
| 116 | 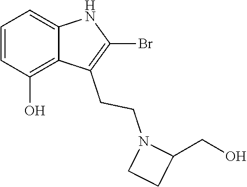 [SMILES: OCC1CCN1CCc1c(Br)[nH]c2cccc(O)c12] | | | | | | |
| 117 | 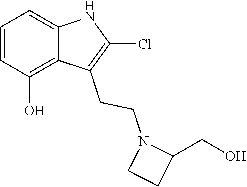 [SMILES: OCC1CCN1CCc1c(Cl)[nH]c2cccc(O)c12] | | | | | | |

### Methods of Treatment

In embodiments, the present disclosure relates a method of treating or preventing neurological disorders in a subject in need thereof, the methods comprising administering a therapeutically effective amount of a compound disclosed herein, a pharmaceutically acceptable salt thereof, or a pharmaceutical composition thereof to a subject.

In embodiments, the neurological disorder is a mood disorder. In embodiments, the mood disorder is clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, major depressive disorder, minor depressive disorder, bipolar disorder or manic depressive disorder, depression caused by chronic medical conditions, treatment-resistant depression, refractory depression, suicidality, suicidal ideation, or suicidal behavior.

In embodiments, the method described herein provides therapeutic effect to a subject suffering from depression (e.g., moderate or severe depression). In embodiments, the mood disorder is associated with neuroendocrine diseases and disorders, neurodegenerative diseases and disorders (e.g., epilepsy), movement disorders, tremor (e.g. Parkinson's Disease), or women's health disorders or conditions. In embodiments, the mood disorder is depression. In embodiments, the mood disorder is treatment-resistant depression or major depressive disorder. In embodiments, the mood disorder is treatment-resistant depression.

In embodiments, the present disclosure provides methods of treating or preventing PTSD, mood disorders, general anxiety disorder, addictive disorders, and/or drug dependence in a subject in need thereof, the methods comprising administering a therapeutically effective amount of a compound disclosed herein, a pharmaceutically acceptable salt thereof, or a pharmaceutical composition thereof to the subject.

In embodiments, the disclosure provides methods of treating or preventing PTSD in a subject in need thereof, the methods comprising administering a therapeutically effective amount of a compound disclosed herein, a pharmaceutically acceptable salt thereof, or a pharmaceutical composition thereof to the subject.

In embodiments, the methods include treating PTSD through induction and maintenance therapy by administering a compound disclosed herein, a pharmaceutically acceptable salt thereof, or a pharmaceutical composition thereof. In some embodiments, the compounds of the present disclosure are used for induction and maintenance therapy to treat PTSD with an improved safety profile when compared to treatment with the entactogenic, oneirophrenic or psychedelic compound (e.g., dimethyltryptamine or related compound, psilocybin or MDMA) alone.

In embodiments, the present disclosure provides methods of treating or preventing behavioral or mood disorders in a subject in need thereof, the methods comprising administering a therapeutically effective amount of a compound disclosed herein, a pharmaceutically acceptable salt thereof, or a pharmaceutical composition thereof to the subject. In embodiments, the behavioral or mood disorder includes anxiety, such as social anxiety in autistic subjects (e.g., autistic adults) and anxiety related to life-threatening illnesses.

In embodiments, the behavioral or mood disorder includes stress (where moderation thereof is measured, e.g., by effects on amygdala responses). In embodiments, the anxiety disorder is panic disorder, obsessive-compulsive disorder, and/or general anxiety disorder. In embodiments, the subject suffers from a lack of motivation, attention, lack of accuracy in memory recall, speed of response, perseveration, and/or cognitive engagement. Further examples include depression (e.g., MDD or TRD), attention disorders, disorders of executive function and/or cognitive engagement, obsessive compulsive disorder, bipolar disorder, panic disorder, phobia, schizophrenia, psychopathy, antisocial personality disorder and/or neurocognitive disorders.

In embodiments, the present disclosure provides methods for treating an addictive disorder in a subject in need thereof, the methods comprising administering a therapeutically effective amount of a compound disclosed herein, a pharmaceutically acceptable salt thereof, or a pharmaceutical composition thereof to the subject. In some embodiments, the addictive disorder is alcohol abuse, substance abuse, smoking, obesity, or combinations thereof. In embodiments, the disorder is an eating disorder (e.g., anorexia nervosa, bulimia nervosa, binge eating disorder, etc.) or an auditory disorder.

In embodiments, the present disclosure provides methods for treating an impulsive disorder in a subject in need thereof, the methods comprising administering a therapeutically effective amount of a compound disclosed herein, a pharmaceutically acceptable salt thereof, or a pharmaceutical composition thereof to the subject. In embodiments, the impulsive disorder is attention deficit hyperactivity disorder (ADHD), attention deficit disorder (ADD), Tourette's syndrome, autism, or combinations thereof.

In embodiments, the present disclosure provides methods for treating a compulsive disorder in a subject in need thereof, the methods comprising administering a therapeutically effective amount of a compound disclosed herein, a pharmaceutically acceptable salt thereof, or a pharmaceutical composition thereof to the subject. In embodiments, the compulsive disorder is obsessive compulsive disorder (OCD), gambling, aberrant sexual behavior, or combinations thereof.

In embodiments, the present disclosure provides methods for treating a personality disorder in a subject in need thereof, the methods comprising administering a therapeutically effective amount of a compound disclosed herein, a pharmaceutically acceptable salt thereof, or a pharmaceutical composition thereof. In embodiments, the personality is conduct disorder, antisocial personality, aggressive behavior, or a combination thereof to the subject.

In embodiments, the present disclosure relates to one of the treatment methods set forth above comprising administering one of the compounds of the invention in an amount effective to elicit a therapeutic effect without causing a euphoric or psychedelic effect.

In embodiments, the present disclosure relates to an oral dosage form comprising one of the above compounds having a PK profile and/or half-life such that, upon administration to a patient, provides a therapeutic effect but converts in vivo to an inactive metabolite prior to the onset of a euphoric or hallucinogenic effect.

In embodiments, the present disclosure relates to an inactive acid metabolite resulting from the in vivo conversion of an ester moiety of one of the compounds set forth above.

### NUMBERED EMBODIMENTS

In addition the disclosure above, the Examples below, and the appended claims, the disclosure sets forth the following numbered embodiments.

### Numbered Embodiments #1

1. A compound of formula (I):

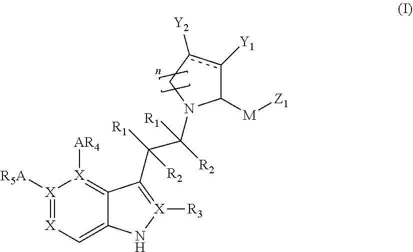

or a pharmaceutically acceptable salt thereof;
wherein,
Y₁ and Y₂ are each independently hydrogen (H), deuterium (D), —CH₃, —OR₁, —CO₂, —NR₁R₂;
Z₁ is —OH, —OD, —SH, —SD, —NR₁R₂, —CN, O-alkyl, O-cycloalkyl, or O-aromatic,
A is a bond, O, N, or S;
each X is independently —CH, —C, or —N;
M is absent, —CH₂—, —C═O, or —CR₁R₂, wherein when M is absent, Z₁ is CN;
R₁, R₂, and R₃ are each independently H, D, alkyl, cycloalkyl, aromatic, or heteroaromatic, wherein R₃ is optionally absent;
R₄ and R₅ are each independently H, D, alkyl, cycloalkyl, aromatic, or heteroaromatic; or
R₄ and R₅ are each independently prodrugs; and
n is 0, 1, or 2, wherein each alkyl, cycloalkyl, aromatic and heteroaromatic is optionally substituted.
   2. The compound of embodiment 1, wherein the alkyl of R₁, R₂, and R₃ is a substituted alkyl.
   3. The compound of embodiment 2, wherein the alkyl is substituted with a heteroatom or halogen.
   4. The compound of embodiment 1, wherein the cycloalkyl of R₁, R₂, and R₃ is a substituted cycloalkyl.
   5. The compound of embodiment 4, wherein the cycloalkyl is substituted with a heteroatom or halogen.
   6. The compound of embodiment 1, wherein the alkyl of R₄ or R₅ is a substituted alkyl.
   7. The compound of embodiment 6, wherein the alkyl is substituted with a heteroatom or halogen.
   8. The compound of embodiment 1, wherein the cycloalkyl of R₄ or R₅ is a substituted cycloalkyl.
   9. The compound of embodiment 8, wherein the cycloalkyl is substituted with a heteroatom or halogen.
   10. The compound of embodiment 1, wherein the pro-drugs are selected from aliphatic ester, aromatic ester, heteroaromatic ester, aminal, hemiaminal, CH₂—OPO₃H₂, and —CH-alkyl-OPO₃H₂.
   11. A compound of Formula (II):

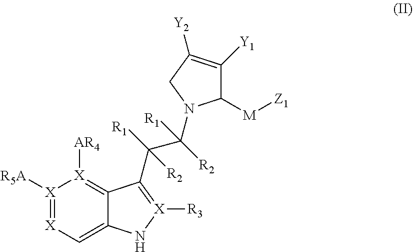

or a pharmaceutically acceptable salt thereof;
wherein,
Y₁ and Y₂ are each independently H, D, —OR₁, —CO₂, or —NR₁R₂;
Z₁ is —OH, —OD, —SH, —SD, —NR₁R₂, O-alkyl, O-cycloalkyl, or O-aromatic;
each A is independently a bond, H, D, O, N, or S;
each X is independently —CH, —C, or —N;
M is —CH₂—, —C═O, or —CR₁R₂;
R₁, R₂, and R₃ are each independently H, D, alkyl, cycloalkyl, aromatic, or heteroaromatic, wherein R₃ is optionally absent; and
R₄ and R₅ are each independently H, D or alkyl, cycloalkyl, aromatic, or heteroaromatic;
or
R₄ and R₅ are prodrugs.
   12. The compound of embodiment 11, wherein the alkyl of R₁, R₂, and R₃ is a substituted alkyl.
   13. The compound of embodiment 12, wherein the alkyl is substituted with a heteroatom or halogen.
   14. The compound of embodiment 11, wherein the cycloalkyl of R₁, R₂, and R₃ is a substituted cycloalkyl.
   15. The compound of embodiment 14, wherein the cycloalkyl is substituted with a heteroatom or halogen.
   16. The compound of embodiment 11, wherein the alkyl of R₄ or R₅ is a substituted alkyl.
   17. The compound of embodiment 16, wherein the alkyl is substituted with a heteroatom or halogen.
   18. The compound of embodiment 11, wherein the cycloalkyl of R₄ or R₅ is a substituted cycloalkyl.
   19. The compound of embodiment 18, wherein the cycloalkyl is substituted with a heteroatom or halogen.
   20. The compound of embodiment 11, wherein the pro-drugs are selected from aliphatic ester, aromatic ester, heteroaromatic ester, aminal, hemiaminal, CH₂—OPO₃H₂, and —CH-alkyl-OPO₃H₂.
   21. A compound of formula III:

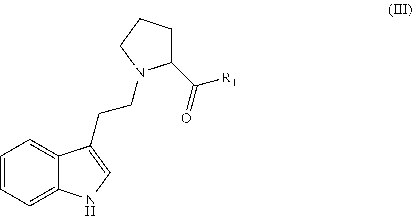

where R₁ is O-alkyl, O-cycloalkyl, or hydroxy.
   22. The compound of embodiment 21, wherein the alkyl is ethyl or methyl.
   23. A compound selected from Table 1.
   24. An inactive metabolite comprising the DMT compound of formula (I) including an ester-containing moiety at the a nitrogen of the compound which, in vivo, metabolizes to an inactive acid metabolite.
   25. A compound of the formula:

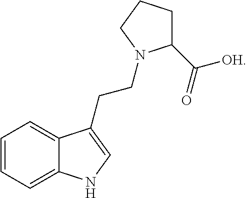 [SMILES: O=C(O)C1CCCN1CCc1c[nH]c2ccccc12]

26. A compound of Formula (IV):

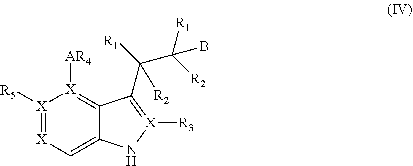

or a pharmaceutically acceptable salt thereof;
wherein,
A is a bond, H, D, O, N, or S;
B is an nitrogen-linked C₅-C₇ heterobicycle;
each X is independently —CH, —C, or —N;
R₁, R₂, and R₃ are each independently H, D, alkyl, cycloalkyl, aromatic, or heteroaromatic, wherein R₃ is optionally absent; and
R₄ and R₅ are each independently H, D or alkyl, cycloalkyl, aromatic, or heteroaromatic; or R₄ and R₅ are prodrugs,
wherein each alkyl, cycloalkyl, aromatic. heteroaromatic, and heterobicycle are optionally substituted.
   27. The compound of embodiment 26, wherein B is a substituted C₅ heterobicycle.
   28. The compound of any one of embodiments 26-27, wherein B is:

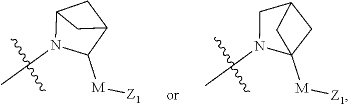

wherein M is absent, —CH₂—, —C═O, or —CR₁R₂; and
Z₁ is —OH, —OD, —SH, —SD, —NR₁R₂, O-alkyl, O-cycloalkyl, or O-aromatic,
wherein when M is absent, Z₁ is CN.
   29. The compound of any one of embodiments 26-28, wherein each X is —CH.
   30. A compound of Formula (V):

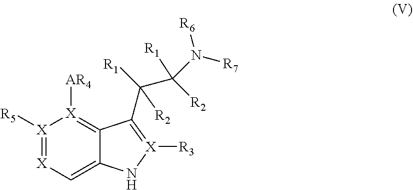

or a pharmaceutically acceptable salt thereof;
wherein,
A is a bond, H, D, O, N, or S;
each X is independently —CH, —C, or —N;
each R₁, R₂, and R₃ are each independently H, D, alkyl, cycloalkyl, aromatic, or heteroaromatic, wherein R₃ is optionally absent; and
R₄ and R₅ are each independently H, D or alkyl, cycloalkyl, aromatic, or heteroaromatic; or R₄ and R₅ are prodrugs,
R₆ is C₁-C₃ alkyl;
R₇ is -(alkyl)COOH, -(alkyl)COOR₁, and C₁-C₃ alkyl,
wherein each alkyl, cycloalkyl, aromatic and heteroaromatic is optionally substituted.
   31. The compound of embodiment 30, wherein each X is —CH.
   32. The compound of any one of embodiments 30-31, wherein R₆ is methyl.

### Numbered Embodiments #2

1. A compound of formula (I-A):

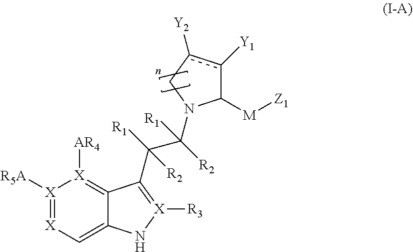

or a pharmaceutically acceptable salt thereof or deuterated form thereof;
wherein,
Y₁ and Y₂ are each independently hydrogen (H), deuterium (D), —CH₃, —OR₁, —CO₂, —NR₁R₂;
Z₁ is —OH, —OD, —SH, —SD, —NR₁R₂, —CN, O-alkyl, O-cycloalkyl, or O-aromatic,
A is a bond, O, N, or S;
each X is independently —CH, —C, or —N;
M is absent, —CH₂—, —C═O, or —CR₁R₂, wherein when M is absent, Z₁ is CN;
R₁, R₂, and R₃ are each independently H, D, alkyl, cycloalkyl, aromatic, halogen, or heteroaromatic, wherein R₃ is optionally absent;
R₄ and R₅ are each independently H, D, alkyl, cycloalkyl, aromatic, halogen substituted alkyl or cycloalkyl, halogen, or heteroaromatic;
or
R₄ and R₅ are each independently prodrugs; and
n is 0, 1, or 2,
wherein each alkyl, cycloalkyl, aromatic and heteroaromatic is optionally substituted.
   2. The compound of embodiment 1, wherein the alkyl of R₁, R₂, and R₃ is a substituted alkyl.
   3. The compound of embodiment 2, wherein the alkyl is substituted with a heteroatom or halogen.
   4. The compound of embodiment 1, wherein the cycloalkyl of R₁, R₂, and R₃ is a substituted cycloalkyl.
   5. The compound of embodiment 4, wherein the cycloalkyl is substituted with a heteroatom or halogen.
   6. The compound of embodiment 1, wherein the alkyl of R₄ or R₅ is a substituted alkyl.
   7. The compound of embodiment 6, wherein the alkyl is substituted with a heteroatom or halogen.
   8. The compound of embodiment 1, wherein the cycloalkyl of R₄ or R₅ is a substituted cycloalkyl.
   9. The compound of embodiment 8, wherein the cycloalkyl is substituted with a heteroatom or halogen.
   10. The compound of embodiment 1, wherein the pro-drugs are selected from aliphatic ester, aromatic ester, heteroaromatic ester, aminal, hemiaminal, —OPO₃H₂, —CH₂—OPO₃H₂, and —CH— alkyl-OPO₃H₂.
   11. A compound of Formula (II-A):

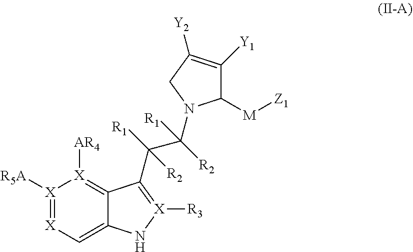

or a pharmaceutically acceptable salt thereof or deuterated form thereof;
wherein,
Y₁ and Y₂ are each independently H, D, —OR₁, —CO₂, or —NR₁R₂;
Z₁ is —OH, —OD, —SH, —SD, —NR₁R₂, O-alkyl, O-cycloalkyl, or O-aromatic;
each A is independently a bond, H, D, O, N, or S;
each X is independently —CH, —C, or —N;
M is —CH₂—, —C═O, or —CR₁R₂;
R₁, R₂, and R₃ are each independently H, D, alkyl, cycloalkyl, aromatic, halogen, or heteroaromatic, wherein R₃ is optionally absent; and
R₄ and R₅ are each independently H, D or alkyl, cycloalkyl, aromatic, halogen, or heteroaromatic; or
R₄ and R₅ are prodrugs.
   12. The compound of embodiment 11, wherein the alkyl of R₁, R₂, and R₃ is a substituted alkyl.
   13. The compound of embodiment 12, wherein the alkyl is substituted with a heteroatom or halogen.
   14. The compound of embodiment 11, wherein the cycloalkyl of R₁, R₂, and R₃ is a substituted cycloalkyl.
   15. The compound of embodiment 14, wherein the cycloalkyl is substituted with a heteroatom or halogen.
   16. The compound of embodiment 11, wherein the alkyl of R₄ or R₅ is a substituted alkyl.
   17. The compound of embodiment 16, wherein the alkyl is substituted with a heteroatom or halogen.
   18. The compound of embodiment 11, wherein the cycloalkyl of R₄ or R₅ is a substituted cycloalkyl.
   19. The compound of embodiment 18, wherein the cycloalkyl is substituted with a heteroatom or halogen.
   20. The compound of embodiment 11, wherein the pro-drugs are selected from aliphatic ester, aromatic ester, heteroaromatic ester, aminal, hemiaminal, —OPO₃H₂, CH₂—OPO₃H₂, and —CH-alkyl-OPO₃H₂.
   21. A compound of formula III:

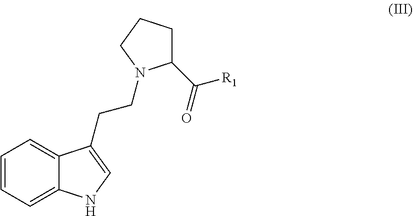

where R₁ is O-alkyl, O-cycloalkyl, or hydroxy.
   22. The compound of embodiment 21, wherein the alkyl is ethyl or methyl.
   23. A compound selected from Table 1.
   24. An inactive metabolite comprising the DMT compound of formula (I) including an ester-containing moiety at the a nitrogen of the compound which, in vivo, metabolizes to an inactive acid metabolite.
   25. A compound of the formula:

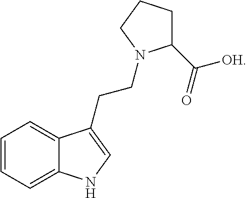 [SMILES: O=C(O)C1CCCN1CCc1c[nH]c2ccccc12]

26. A compound of Formula (IV):

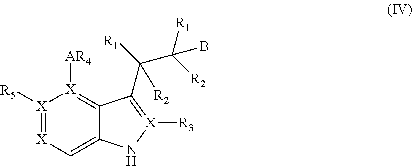

or a pharmaceutically acceptable salt thereof;
wherein,
A is a bond, H, D, O, N, or S;
B is an nitrogen-linked C₅-C₇ heterobicycle;
each X is independently —CH, —C, or —N;
R₁, R₂, and R₃ are each independently H, D, alkyl, cycloalkyl, aromatic, or heteroaromatic, wherein R₃ is optionally absent; and
R₄ and R₅ are each independently H, D or alkyl, cycloalkyl, aromatic, or heteroaromatic;
or R₄ and R₅ are prodrugs,
wherein each alkyl, cycloalkyl, aromatic, heteroaromatic, and heterobicycle are optionally substituted.
   27. The compound of embodiment 26, wherein B is a substituted C₅ heterobicycle.
   28. The compound of any one of embodiments 26-27, wherein B is:

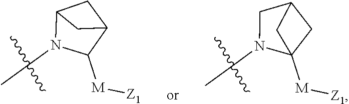

wherein M is absent, —CH₂—, —C═O, or —CR₁R₂; and
Z₁ is —OH, —OD, —SH, —SD, —NR₁R₂, O-alkyl, O-cycloalkyl, or O-aromatic,
wherein when M is absent, Z₁ is CN.
   29. The compound of any one of embodiments 26-28, wherein each X is —CH.
   30. A compound of Formula (V):

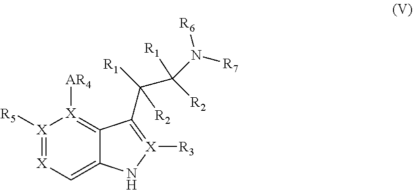

or a pharmaceutically acceptable salt thereof;
wherein,
A is a bond, H, D, O, N, or S;
each X is independently —CH, —C, or —N;
each R₁, R₂, and R₃ are each independently H, D, alkyl, cycloalkyl, aromatic, or heteroaromatic, wherein R₃ is optionally absent; and
R₄ and R₅ are each independently H, D or alkyl, cycloalkyl, aromatic, or heteroaromatic;
or R₄ and R₅ are prodrugs,
R₆ is C₁-C₃ alkyl;
R₇ is -(alkyl)COOH, -(alkyl)COOR₁, and C₁-C₃ alkyl,
wherein each alkyl, cycloalkyl, aromatic and heteroaromatic is optionally substituted.
   31. The compound of embodiment 30, wherein each X is —CH.
   32. The compound of any one of embodiments 30-31, wherein R₆ is methyl.
   33. A compound of Formula (I-B)

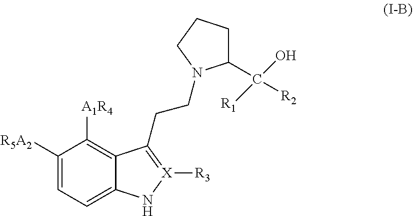

or a pharmaceutically acceptable salt or deuterated form thereof;
wherein,
A₁ and A₂ are each independently a bond, H, D, O, N, or S;
R₁ and R₂ are each independently H or alkyl;
R₃ is H, D, alkyl, cycloalkyl, aromatic, halogen, heteroaromatic, or absent;
R₄ and R₅ are each independently H, D, alkyl, cycloalkyl, aromatic, halogen substituted alkyl or cycloalkyl, halogen, or heteroaromatic, or
R₄ and R₅ are each independently a prodrug; and
X is independently —CH, —C, or —N.
   34. The compound of embodiment 33, wherein R₁ is H.
   35. The compound of embodiment 33, wherein R₁ is alkyl.
   36. The compound of any one of embodiments 33-35, wherein R₂ is H.
   37. The compound of any one of embodiments 33-35, wherein R₂ is alkyl.
   38. The compound of any one of embodiments 33-37, wherein A₁ is a bond.
   39. The compound of any one of embodiments 33-37, wherein A₁ is O.
   40. The compound of any one of embodiments 33-39, wherein A₂ is a bond.
   41. The compound of any one of embodiments 33-39, wherein A₂ is O.
   42. The compound of any one of embodiments 33-41, wherein R₃ absent.
   43. The compound of any one of embodiments 33-41, wherein R₃ is halogen.
   44. The compound of any one of embodiments 33-41, wherein R₃ is F, Cl, or Br.
   45. The compound of any one of embodiments 33-44, wherein R₄ is H.
   46. The compound of any one of embodiments 33-44, wherein R₄ is an alkyl.
   47. The compound of embodiment 46, wherein the alkyl is —CH₃.
   48. The compound of any one of embodiments 33-44, wherein R₄ is a halogen substituted alkyl.
   49. The compound of embodiment 48, wherein the halogen substituted alkyl is —CF₃.
   50. The compound of any one of embodiments 33-44, wherein R₄ is a halogen.
   51. The compound of embodiment 50, wherein R₄ is F, Cl, or Br.
   52. The compound of any one of embodiments 33-51, wherein R₅ is H.
   53. The compound of any one of embodiments 33-51, wherein R₅ is an alkyl.
   54. The compound of embodiment 53, wherein the alkyl is —CH₃.
   55. The compound of any one of embodiments 33-51, wherein R₅ is a halogen substituted alkyl.
   56. The compound of embodiment 55, wherein the halogen substituted alkyl is —CF₃.
   57. The compound of any one of embodiments 33-51, wherein R₅ is a halogen.
   58. The compound of embodiment 57, wherein R₅ is F, Cl, or Br.
   59. The compound of any one of embodiments 33-44, wherein at least one of R₄ or R₅ is a prodrug.
   60. The compound of embodiment 59, wherein the prodrug is an aliphatic ester, aromatic ester, heteroaromatic ester, aminal, hemiaminal, —OPO₃H₂, —CH₂—OPO₃H₂, and —CH-alkyl-OPO₃H₂.
   61. The compound of any one of embodiments 33-60, wherein X is CH.
   62. The compound of embodiment 61, wherein R₃ is absent.
   63. The compound of any one of embodiments 33-58, wherein X is —C.
   64. The compound of embodiment 63, wherein R₃ is halogen.
   65. The compound of embodiment 33, wherein A₁ is a bond and R₄ is H.
   66. The compound of embodiment 33, wherein A₁ is a bond and R₄ is a halogen.
   67. The compound of embodiment 33, wherein A₁ is a O and R₄ is an alkyl.
   68. The compound of embodiment 67, wherein the alkyl is —CH₃.
   69. The compound of embodiment 33, wherein A₁ is O and R₄ is a halogen substituted alkyl.
   70. The compound of embodiment 69, wherein the halogen substituted alkyl is —CF₃.
   71. The compound of embodiment 33, wherein A₁ is a bond and R₅ is H.
   72. The compound of embodiment 33, wherein A₁ is a bond and R₅ is a halogen.
   73. The compound of embodiment 33, wherein A₁ is a O and R₅ is an alkyl.
   74. The compound of embodiment 73, wherein the alkyl is —CH₃.
   75. The compound of embodiment 33, wherein A₁ is O and R₅ is a halogen substituted alkyl.
   76. The compound of embodiment 75, wherein the halogen substituted alkyl is —CF₃.
   77. The compound of any one of embodiments 33-76, wherein the compounds is:

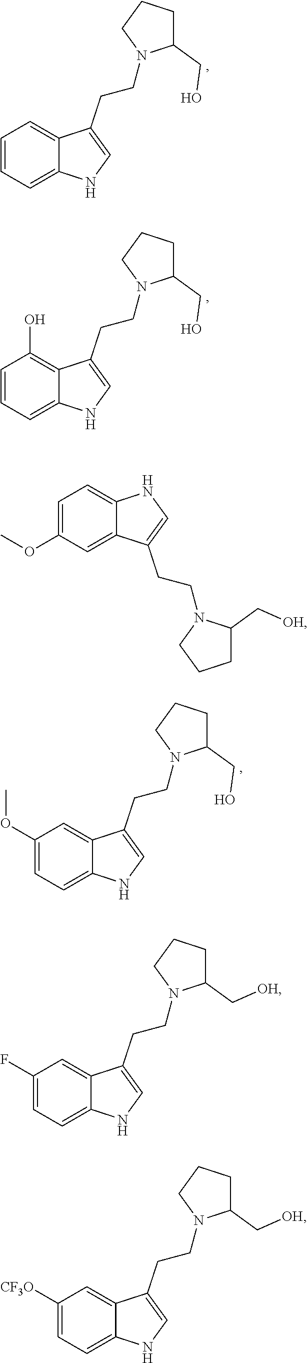

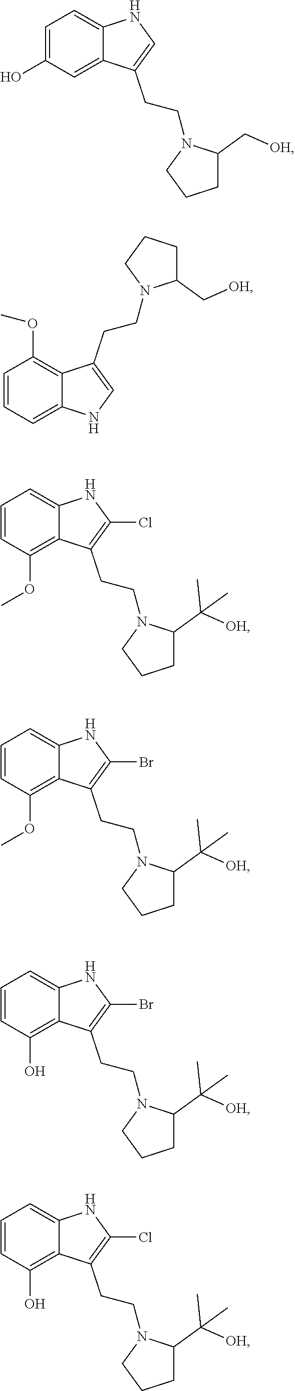 [SMILES: OCC1CCCN1CCc1c[nH]c2ccc(O)cc12 ; COc1cccc2[nH]cc(CCN3CCCC3CO)c12 ; COc1cccc2[nH]c(Cl)c(CCN3CCCC3C(C)(C)O)c12 ; COc1cccc2[nH]c(Br)c(CCN3CCCC3C(C)(C)O)c12 ; CC(C)(O)C1CCCN1CCc1c(Br)[nH]c2cccc(O)c12 ; CC(C)(O)C1CCCN1CCc1c(Cl)[nH]c2cccc(O)c12]

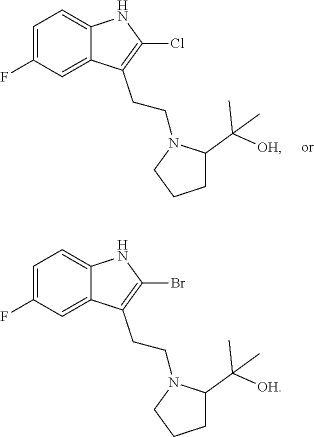 [SMILES: CC(C)(O)C1CCCN1CCc1c(Cl)[nH]c2ccc(F)cc12 ; CC(C)(O)C1CCCN1CCc1c(Br)[nH]c2ccc(F)cc12]

78. The compound of any one of embodiments 33-76, wherein the compounds is:

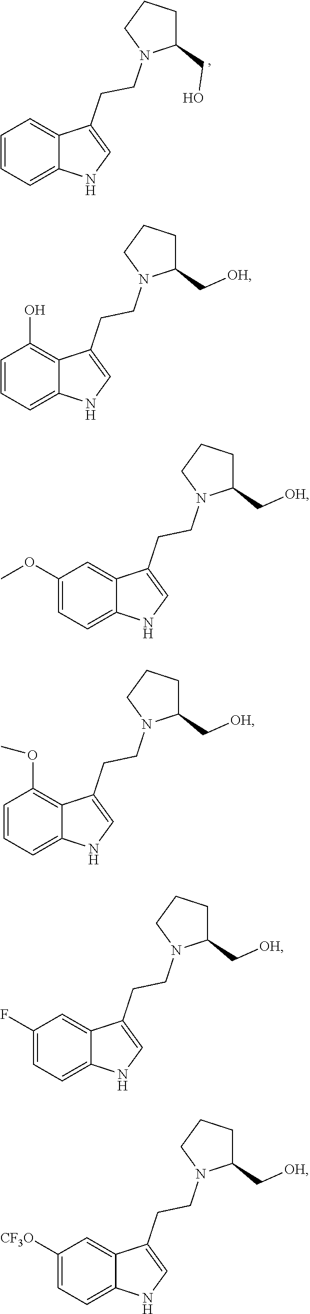

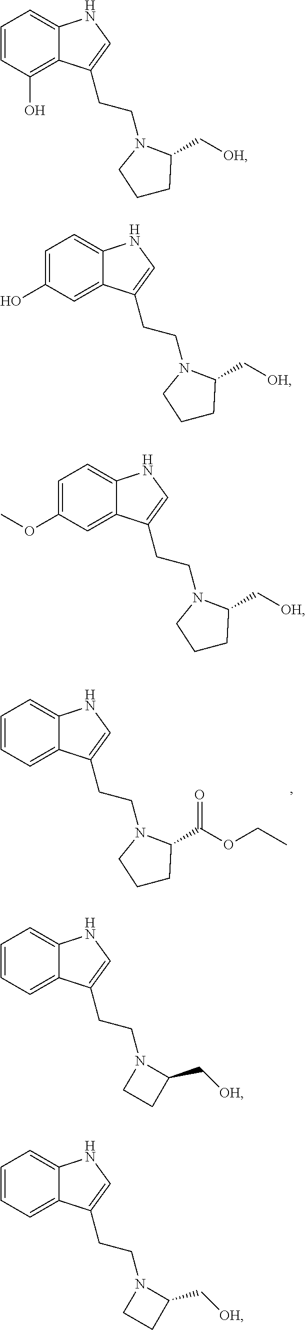

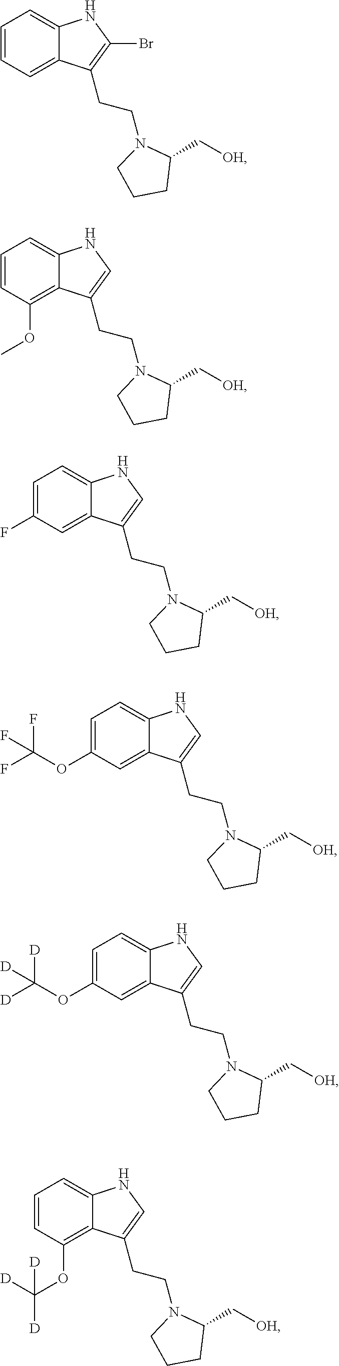 [SMILES: OC[C@@H]1CCCN1CCc1c(Br)[nH]c2ccccc12 ; COc1cccc2[nH]cc(CCN3CCC[C@H]3CO)c12 ; OC[C@@H]1CCCN1CCc1c[nH]c2ccc(F)cc12 ; OC[C@@H]1CCCN1CCc1c[nH]c2ccc(OC(F)(F)F)cc12 ; [2H]C([2H])([2H])Oc1ccc2[nH]cc(CCN3CCC[C@H]3CO)c2c1 ; [2H]C([2H])([2H])Oc1cccc2[nH]cc(CCN3CCC[C@H]3CO)c12]

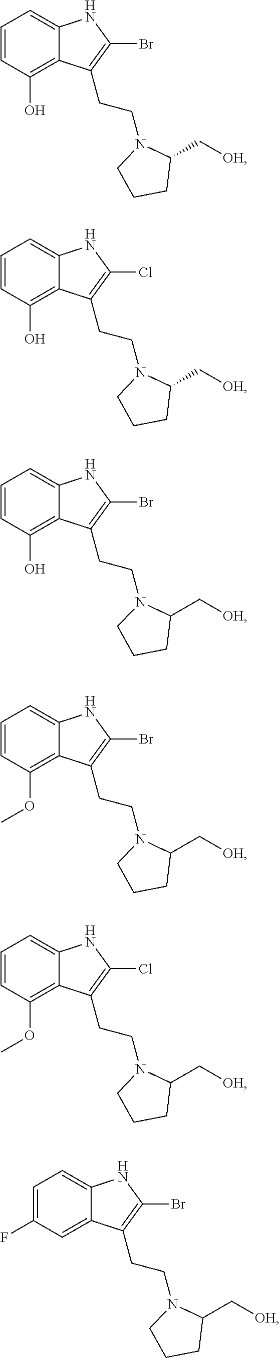 [SMILES: OC[C@@H]1CCCN1CCc1c(Br)[nH]c2cccc(O)c12 ; OC[C@@H]1CCCN1CCc1c(Cl)[nH]c2cccc(O)c12 ; OCC1CCCN1CCc1c(Br)[nH]c2cccc(O)c12 ; COc1cccc2[nH]c(Br)c(CCN3CCCC3CO)c12 ; COc1cccc2[nH]c(Cl)c(CCN3CCCC3CO)c12 ; OCC1CCCN1CCc1c(Br)[nH]c2ccc(F)cc12]

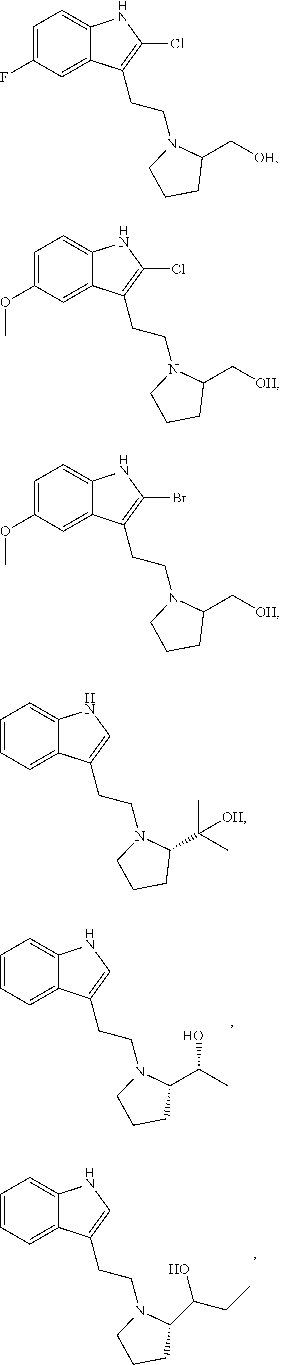 [SMILES: OCC1CCCN1CCc1c(Cl)[nH]c2ccc(F)cc12 ; COc1ccc2[nH]c(Cl)c(CCN3CCCC3CO)c2c1 ; COc1ccc2[nH]c(Br)c(CCN3CCCC3CO)c2c1 ; CC(C)(O)[C@@H]1CCCN1CCc1c[nH]c2ccccc12 ; C[C@@H](O)[C@@H]1CCCN1CCc1c[nH]c2ccccc12 ; CCC(O)[C@@H]1CCCN1CCc1c[nH]c2ccccc12]

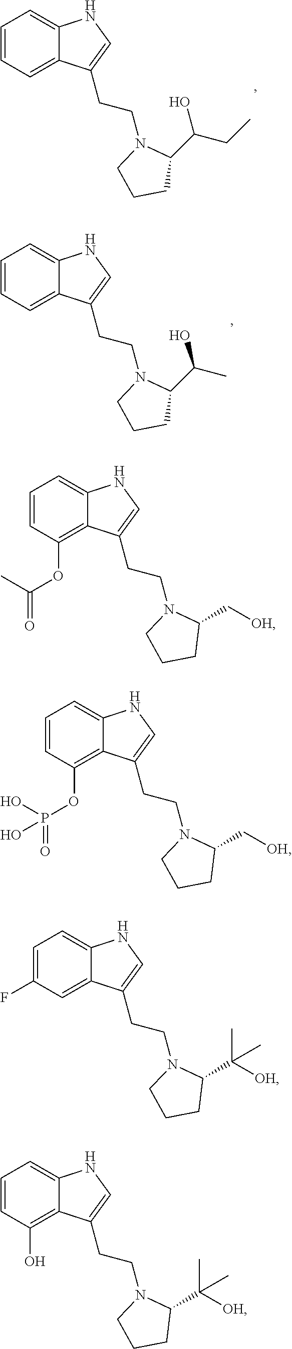

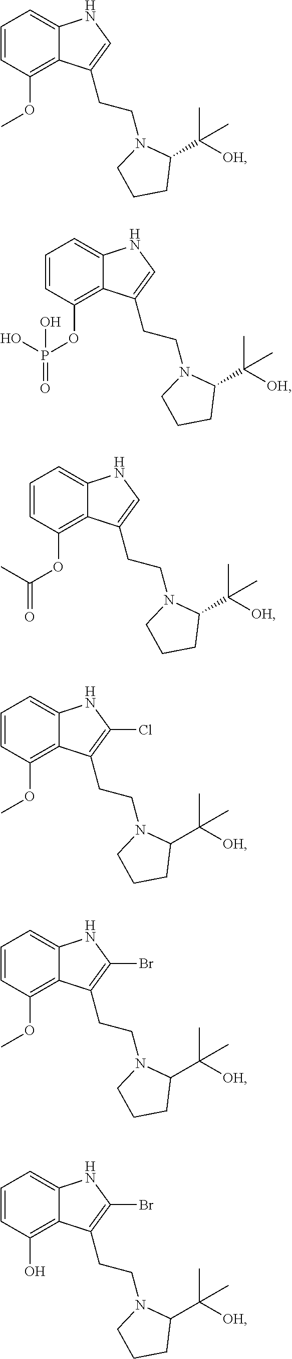

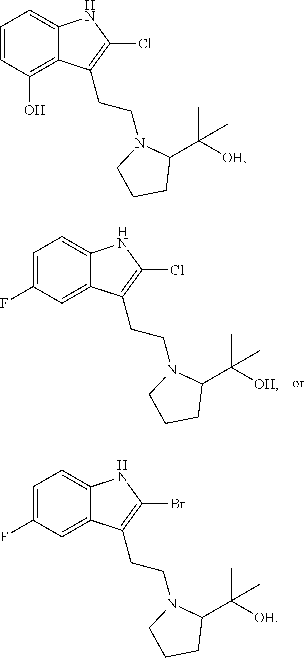 [SMILES: CC(C)(O)C1CCCN1CCc1c(Cl)[nH]c2cccc(O)c12 ; CC(C)(O)C1CCCN1CCc1c(Cl)[nH]c2ccc(F)cc12 ; "or" ; CC(C)(O)C1CCCN1CCc1c(Br)[nH]c2ccc(F)cc12]

### Numbered Embodiments #3

1. A compound of Formula (I-C)

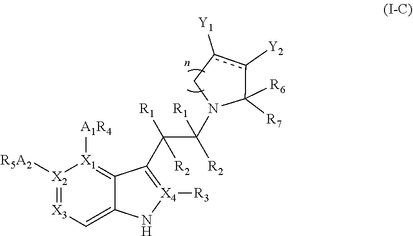

or a pharmaceutically acceptable salt or deuterated form thereof;
wherein,
Y₁ and Y₂ are each independently hydrogen (H), deuterium (D), —CH₃, —OR₁, —CO₂R₈, —NR₁₀R₁₁;
X₁, X₂, X₃, or X₄ are each independently —CH, —C, or —N;
A₁ and A₂ are each independently a bond, H, D, O, N, or S; R₁, R₂, and R₃ are each independently H, D, alkyl, cycloalkyl, aromatic, halogen, heteroaromatic, or absent;
R₄ and R₅ are each independently H, D, alkyl, cycloalkyl, aromatic, halogen substituted alkyl or cycloalkyl, halogen, heteroaromatic, —C(O)CH₃, or —P(O)(OR₉)₂;
R₆ is H or alkyl;
R₇ is

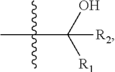

—CO₂R₈, —CH₂C(O)CH₃, —CH₂CO₂R₈, —OP(O)(OR₉)₂, —C(O)—NR₁₀R₁₁;
R₈ is H, alkyl, cycloalkyl, haloalkyl, alkyl-O-alkyl, or aryl;
R₉ is H or alkyl; R₁₀ and R₁₁ are each independently H or alkyl; and
n is 0, 1, or 2, wherein the compound is not

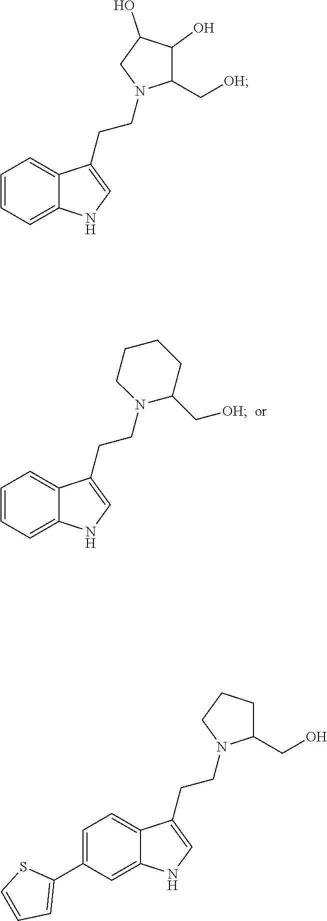

and wherein when n is 2, R⁷ is not —CO₂R₈ or —CH₂CO₂R₈.
   2. The compound of embodiment 1, wherein Y₁ and Y₂ are each H.
   3. The compound of embodiment 1 or 2, wherein X₁ is —C.
   4. The compound of embodiment 1 or 2, wherein X₁ is N.
   5. The compound of any one of embodiments 1-4, wherein X₂ is —C.
   6. The compound of any one of embodiments 1-5, wherein X₃ is N.
   7. The compound of any one of embodiments 1-6, wherein X₄ is —C.
   8. The compound of any one of embodiments 1-7, wherein A₁ is O.
   9. The compound of any one of embodiments 1-8, wherein A₂ is O.
   10. The compound of any one of embodiments 1-9, wherein R₁ is H.
   11. The compound of any one of embodiments 1-10, wherein R₂ is H.
   12. The compound of any one of embodiments 1-11, wherein R₃ is H.
   13. The compound of any one of embodiments 1-11, wherein R₃ is halogen.
   14. The compound of any one of embodiments 1-13, wherein R₄ is H.
   15. The compound of any one of embodiments 1-13, wherein R₅ is H.
   16. The compound of any one of embodiments 1-13, wherein A₁ is O and R₄ is H.
   17. The compound of any one of embodiments 1-13, wherein A₂ is O and R₅ is H.
   18. The compound of any one of embodiments 1-13, wherein A₁ is O and R₄ is —P(O)(OR₉)₂.
   19. The compound of any one of embodiments 1-13, wherein A₂ is O and R₅ is —P(O)(OR₉)₂.
   20. The compound of any one of embodiments 1-13, wherein A₁ is a bond and R₅ is halogen.
   21. The compound of any one of embodiments 1-13, wherein A₂ is a bond and R₄ is halogen.
   22. The compound of any one of embodiments 1-13, wherein A₁ is O and R₄ is H.
   23. The compound of any one of embodiments 1-13, wherein A₂ is O and R₅ is H.
   24. The compound of any one of embodiments 1-13, wherein A₁ is O and R₄ is alkyl.
   25. The compound of any one of embodiments 1-13, wherein A₂ is O and R₅ is alkyl.
   26. The compound of any one of embodiments 1-13, wherein A₁ is O and R₄ is —C(O)CH₃.
   27. The compound of any one of embodiments 1-13, wherein A₂ is O and R₅ is —C(O)CH₃.
   28. The compound of any one of embodiments 1-11, wherein R₆ is H.
   29. The compound of any one of embodiments 1-12, wherein R₆ is alkyl.
   30. The compound of embodiment 29, wherein the alkyl is methyl.
   31. The compound of any one of embodiments 1-30, wherein R₇ is

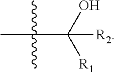

32. The compound of embodiment 31, wherein R₁ and R₂ are each H.
33. The compound of embodiment 31, wherein at least one of R₁ or R₂ is alkyl.
34. The compound of embodiment 31, wherein one of R₁ or R₂ is alkyl and one of R₁ or R₂ is H.
35. The compound of any one of embodiments 1-30, wherein R₁ is —CO₂R₈.
36. The compound of embodiment 35, wherein R₈ is H.
37. The compound of embodiment 35, wherein R₈ is alkyl.
38. The compound of any one of embodiments 1-30, wherein R₁ is —CH₂C(O)CH₃.
39. The compound of any one of embodiments 1-30, wherein R₁ is —CH₂CO₂R₈.
40. The compound of embodiment 39, wherein R₈ is H.
41. The compound of embodiment 39, wherein R₈ is alkyl.
42. The compound of any one of embodiments 1-30, wherein R₁ is —OP(O)(OR₉)₂.
43. The compound of embodiment 42, wherein R₉ is H.
44. The compound of embodiment 42, wherein R₉ is alkyl.
45. The compound of any one of embodiments 1-30, wherein R₁ is —C(O)—NR₁₀R₁₁.
46. The compound of embodiment 45, wherein R₁₀ and R₁₁ are each H.
47. The compound of embodiment 45, wherein at least one of R₁₀ or R₁₁ are alkyl.
48. The compound of embodiment 45, wherein one of R₁₀ or R₁₁ is alkyl and one of R₁₀ or R₁₁ is H.
49. The compound of any one of embodiments 1-48, wherein n is 0.
50. The compound of any one of embodiments 1-48, wherein n is 1.
51. The compound of any one of embodiments 1-48, wherein n is 2.
52. The compound of any one of embodiments 1-48, wherein the compound is:

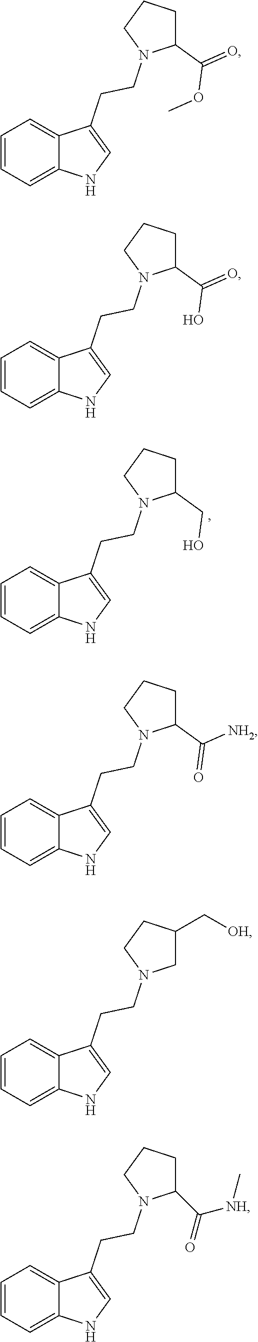 [SMILES: COC(=O)C1CCCN1CCc1c[nH]c2ccccc12 ; O=C(O)C1CCCN1CCc1c[nH]c2ccccc12 ; OCC1CCCN1CCc1c[nH]c2ccccc12 ; NC(=O)C1CCCN1CCc1c[nH]c2ccccc12 ; OCC1CCN(CCc2c[nH]c3ccccc23)C1 ; CNC(=O)C1CCCN1CCc1c[nH]c2ccccc12]

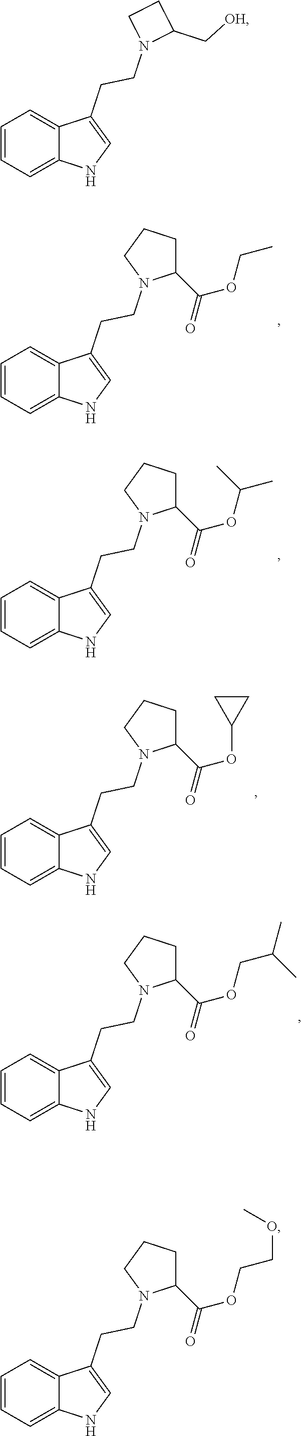 [SMILES: OCC1CCN1CCc1c[nH]c2ccccc12 ; CCOC(=O)C1CCCN1CCc1c[nH]c2ccccc12 ; CC(C)OC(=O)C1CCCN1CCc1c[nH]c2ccccc12 ; O=C(OC1CC1)C1CCCN1CCc1c[nH]c2ccccc12 ; CC(C)COC(=O)C1CCCN1CCc1c[nH]c2ccccc12 ; COCCOC(=O)C1CCCN1CCc1c[nH]c2ccccc12]

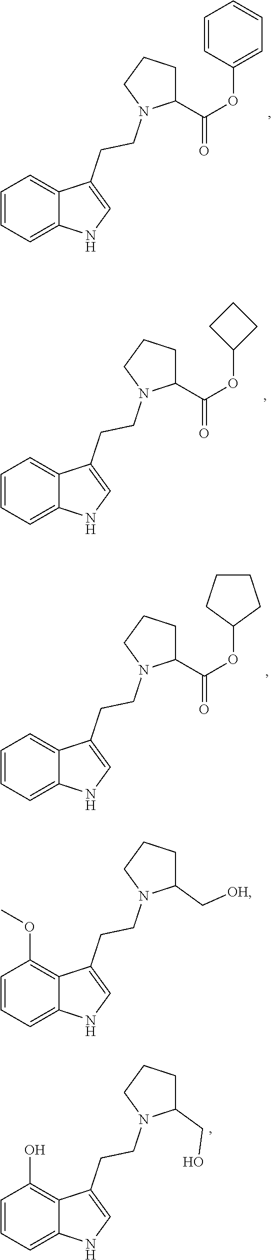 [SMILES: O=C(Oc1ccccc1)C1CCCN1CCc1c[nH]c2ccccc12 ; O=C(OC1CCC1)C1CCCN1CCc1c[nH]c2ccccc12 ; O=C(OC1CCCC1)C1CCCN1CCc1c[nH]c2ccccc12 ; COc1cccc2[nH]cc(CCN3CCCC3CO)c12 ; OCC1CCCN1CCc1c[nH]c2cccc(O)c12]

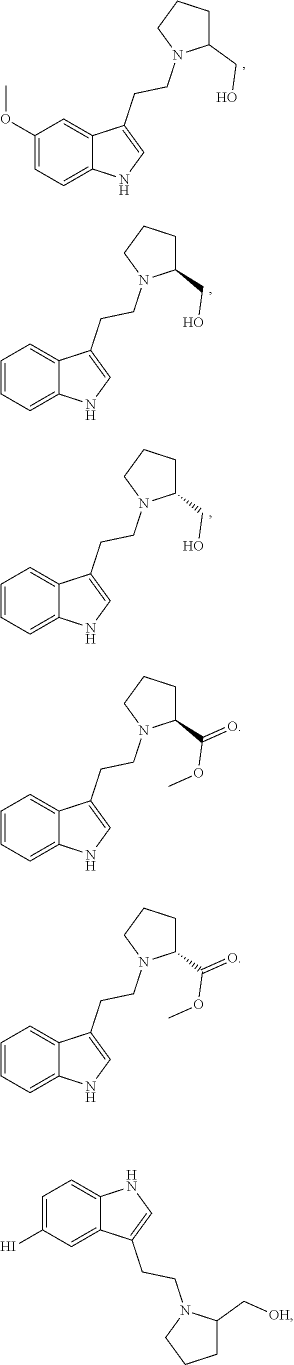 [SMILES: COc1ccc2[nH]cc(CCN3CCCC3CO)c2c1 ; OC[C@@H]1CCCN1CCc1c[nH]c2ccccc12 ; OC[C@H]1CCCN1CCc1c[nH]c2ccccc12 ; COC(=O)[C@@H]1CCCN1CCc1c[nH]c2ccccc12 ; COC(=O)[C@H]1CCCN1CCc1c[nH]c2ccccc12 ; OCC1CCCN1CCc1c[nH]c2ccc(I)cc12]

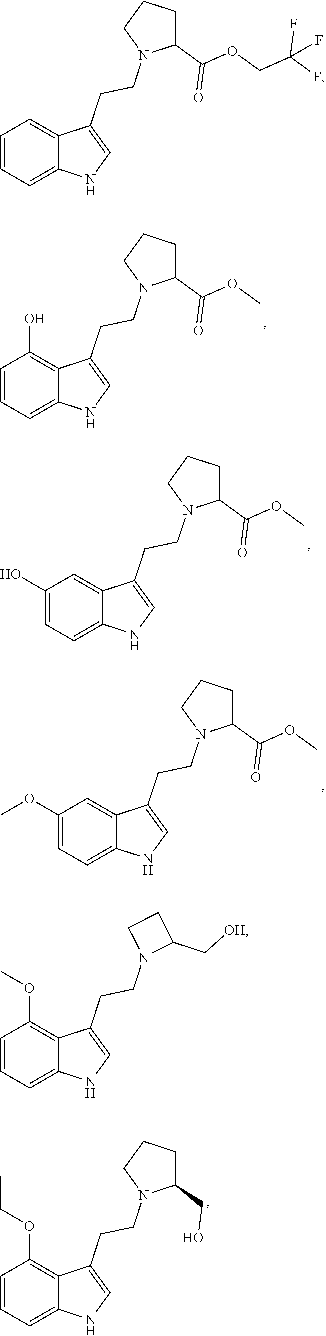

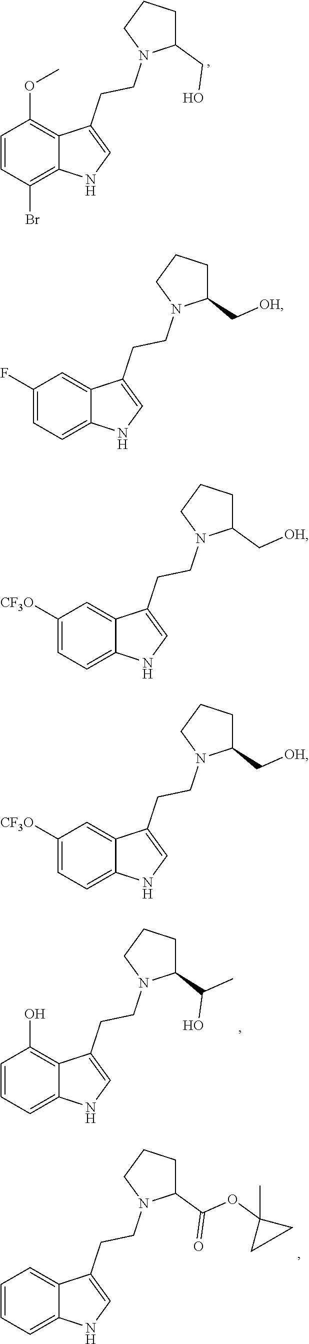

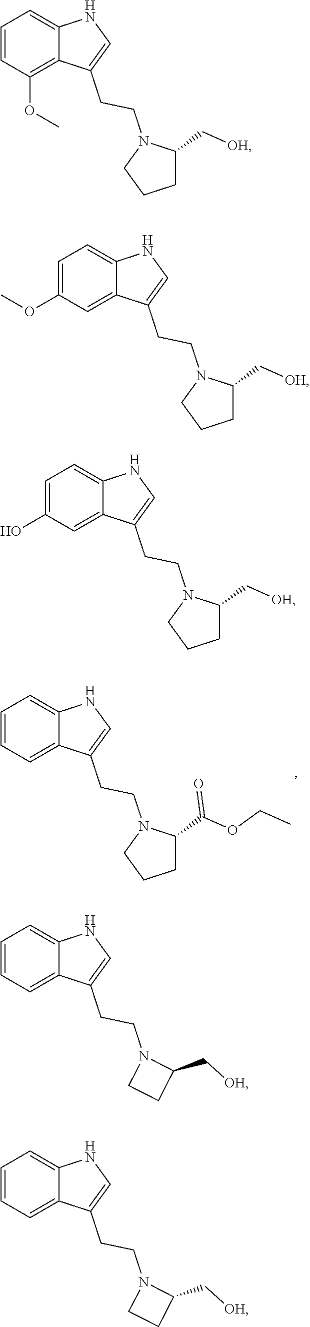 [SMILES: COc1cccc2[nH]cc(CCN3CCC[C@H]3CO)c12 ; COc1ccc2[nH]cc(CCN3CCC[C@H]3CO)c2c1 ; OC[C@@H]1CCCN1CCc1c[nH]c2ccc(O)cc12 ; CCOC(=O)[C@@H]1CCCN1CCc1c[nH]c2ccccc12 ; OC[C@H]1CCN1CCc1c[nH]c2ccccc12 ; OC[C@@H]1CCN1CCc1c[nH]c2ccccc12]

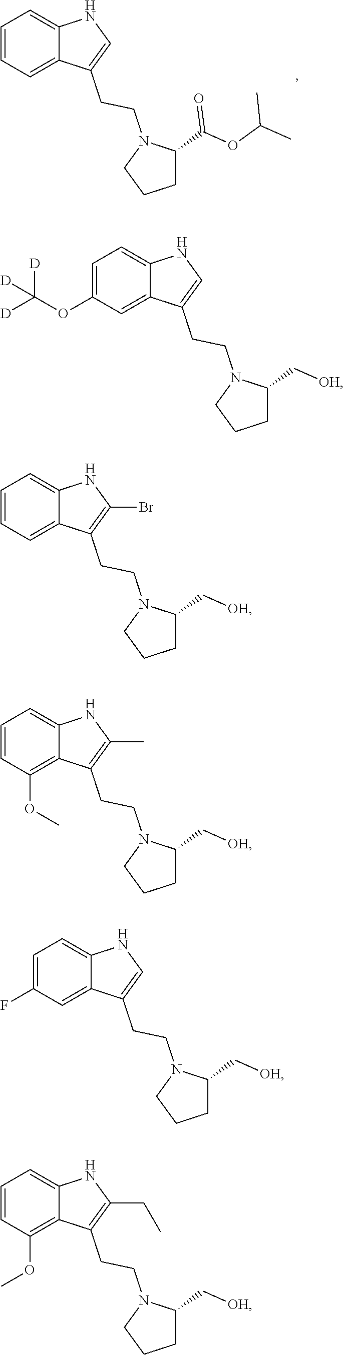 [SMILES: CC(C)OC(=O)[C@@H]1CCCN1CCc1c[nH]c2ccccc12 ; [2H]C([2H])([2H])Oc1ccc2[nH]cc(CCN3CCC[C@H]3CO)c2c1 ; OC[C@@H]1CCCN1CCc1c(Br)[nH]c2ccccc12 ; COc1cccc2[nH]c(C)c(CCN3CCC[C@H]3CO)c12 ; OC[C@@H]1CCCN1CCc1c[nH]c2ccc(F)cc12 ; CCc1[nH]c2cccc(OC)c2c1CCN1CCC[C@H]1CO]

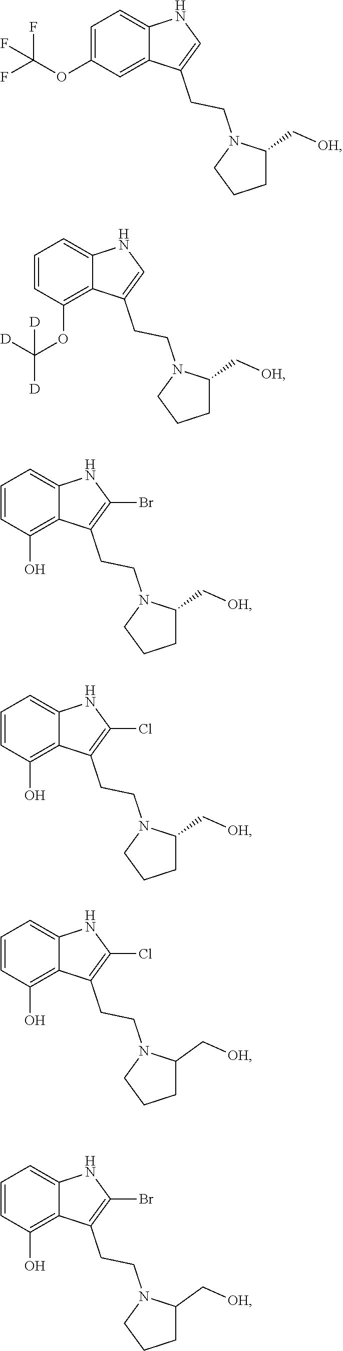 [SMILES: OC[C@@H]1CCCN1CCc1c[nH]c2ccc(OC(F)(F)F)cc12 ; [2H]C([2H])([2H])Oc1cccc2[nH]cc(CCN3CCC[C@H]3CO)c12 ; OC[C@@H]1CCCN1CCc1c(Br)[nH]c2cccc(O)c12 ; OC[C@@H]1CCCN1CCc1c(Cl)[nH]c2cccc(O)c12 ; OCC1CCCN1CCc1c(Cl)[nH]c2cccc(O)c12 ; OCC1CCCN1CCc1c(Br)[nH]c2cccc(O)c12]

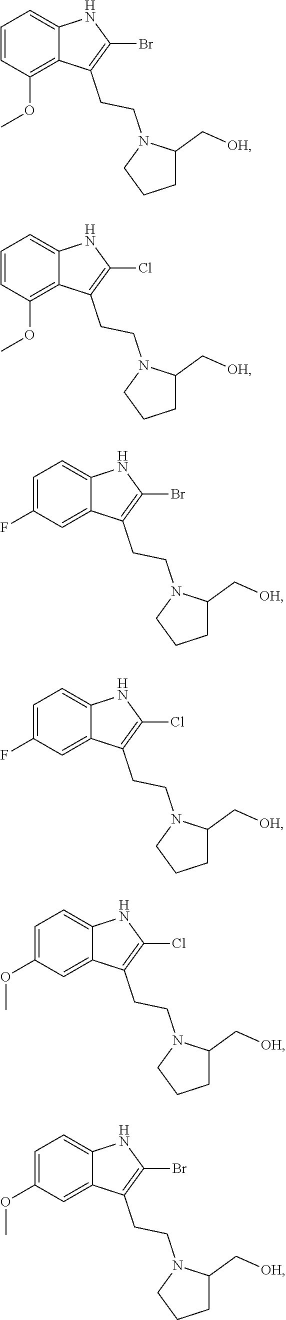 [SMILES: COc1cccc2[nH]c(Br)c(CCN3CCCC3CO)c12 ; COc1cccc2[nH]c(Cl)c(CCN3CCCC3CO)c12 ; OCC1CCCN1CCc1c(Br)[nH]c2ccc(F)cc12 ; OCC1CCCN1CCc1c(Cl)[nH]c2ccc(F)cc12 ; COc1ccc2[nH]c(Cl)c(CCN3CCCC3CO)c2c1 ; COc1ccc2[nH]c(Br)c(CCN3CCCC3CO)c2c1]

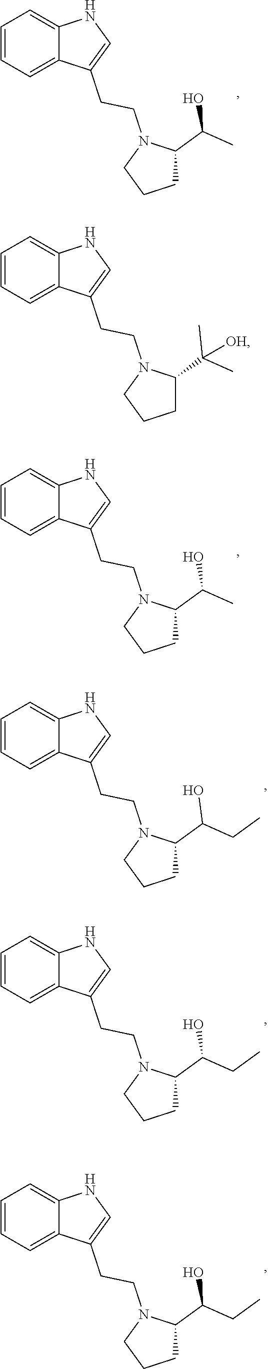 [SMILES: C[C@H](O)[C@@H]1CCCN1CCc1c[nH]c2ccccc12 ; CC(C)(O)[C@@H]1CCCN1CCc1c[nH]c2ccccc12 ; C[C@@H](O)[C@@H]1CCCN1CCc1c[nH]c2ccccc12 ; CCC(O)[C@@H]1CCCN1CCc1c[nH]c2ccccc12 ; CC[C@@H](O)[C@@H]1CCCN1CCc1c[nH]c2ccccc12 ; CC[C@H](O)[C@@H]1CCCN1CCc1c[nH]c2ccccc12]

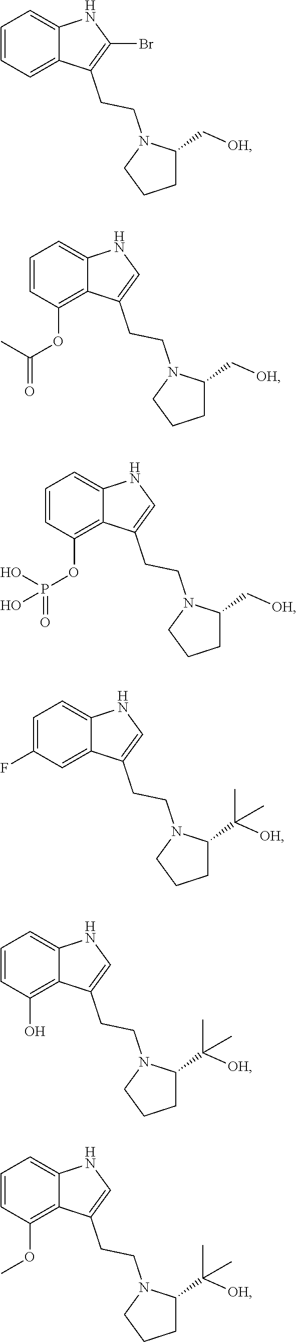 [SMILES: OC[C@@H]1CCCN1CCc1c(Br)[nH]c2ccccc12 ; CC(=O)Oc1cccc2[nH]cc(CCN3CCC[C@H]3CO)c12 ; O=P(O)(O)Oc1cccc2[nH]cc(CCN3CCC[C@H]3CO)c12 ; CC(C)(O)[C@@H]1CCCN1CCc1c[nH]c2ccc(F)cc12 ; CC(C)(O)[C@@H]1CCCN1CCc1c[nH]c2cccc(O)c12 ; COc1cccc2[nH]cc(CCN3CCC[C@H]3C(C)(C)O)c12]

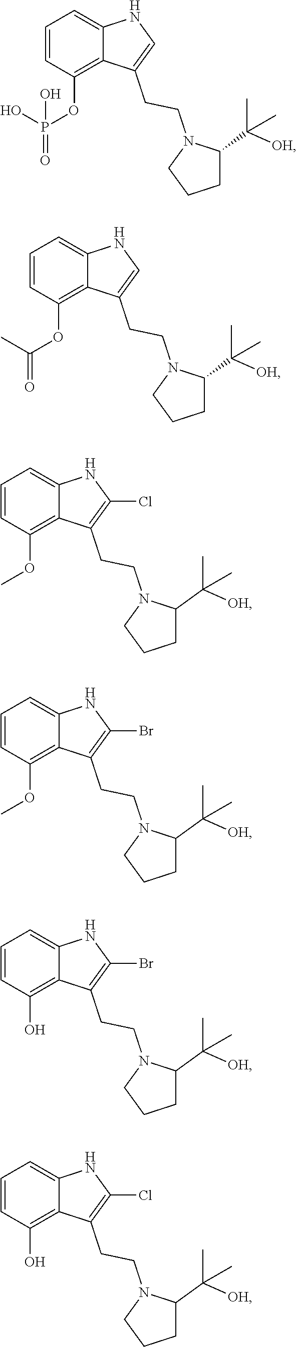 [SMILES: CC(C)(O)[C@@H]1CCCN1CCc1c[nH]c2cccc(OP(=O)(O)O)c12 ; CC(=O)Oc1cccc2[nH]cc(CCN3CCC[C@H]3C(C)(C)O)c12 ; COc1cccc2[nH]c(Cl)c(CCN3CCCC3C(C)(C)O)c12 ; COc1cccc2[nH]c(Br)c(CCN3CCCC3C(C)(C)O)c12 ; CC(C)(O)C1CCCN1CCc1c(Br)[nH]c2cccc(O)c12 ; CC(C)(O)C1CCCN1CCc1c(Cl)[nH]c2cccc(O)c12]

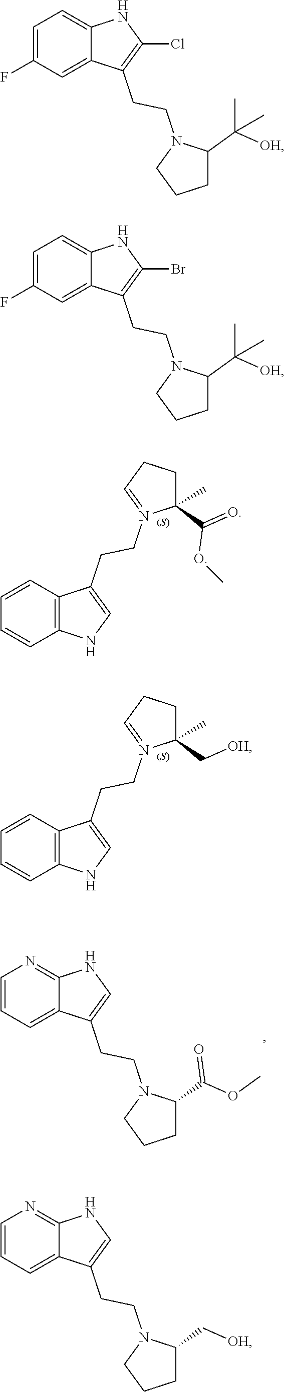

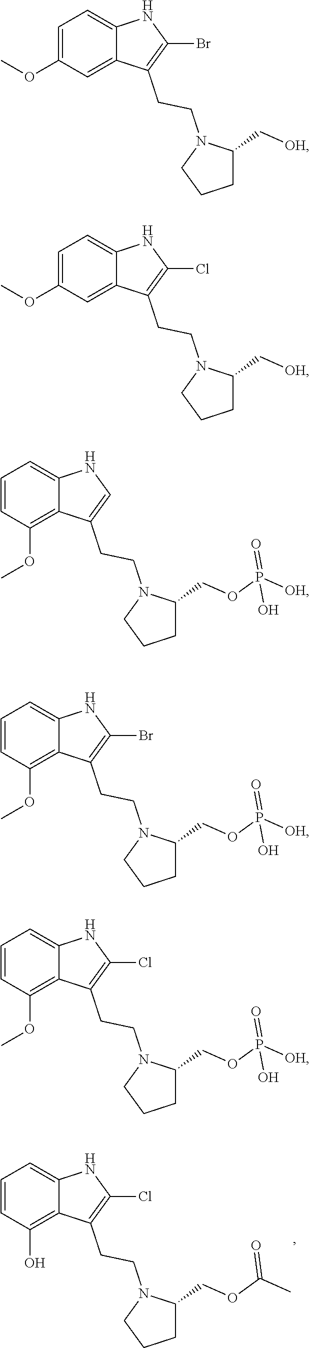 [SMILES: COc1ccc2[nH]c(Br)c(CCN3CCC[C@H]3CO)c2c1 ; COc1ccc2[nH]c(Cl)c(CCN3CCC[C@H]3CO)c2c1 ; COc1cccc2[nH]cc(CCN3CCC[C@H]3COP(=O)(O)O)c12 ; COc1cccc2[nH]c(Br)c(CCN3CCC[C@H]3COP(=O)(O)O)c12 ; COc1cccc2[nH]c(Cl)c(CCN3CCC[C@H]3COP(=O)(O)O)c12 ; CC(=O)OC[C@@H]1CCCN1CCc1c(Cl)[nH]c2cccc(O)c12]

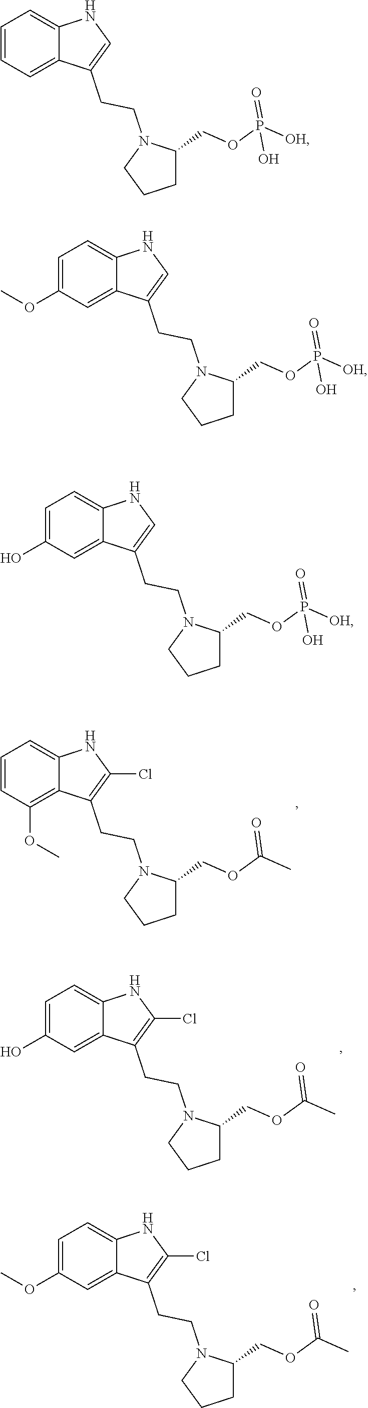

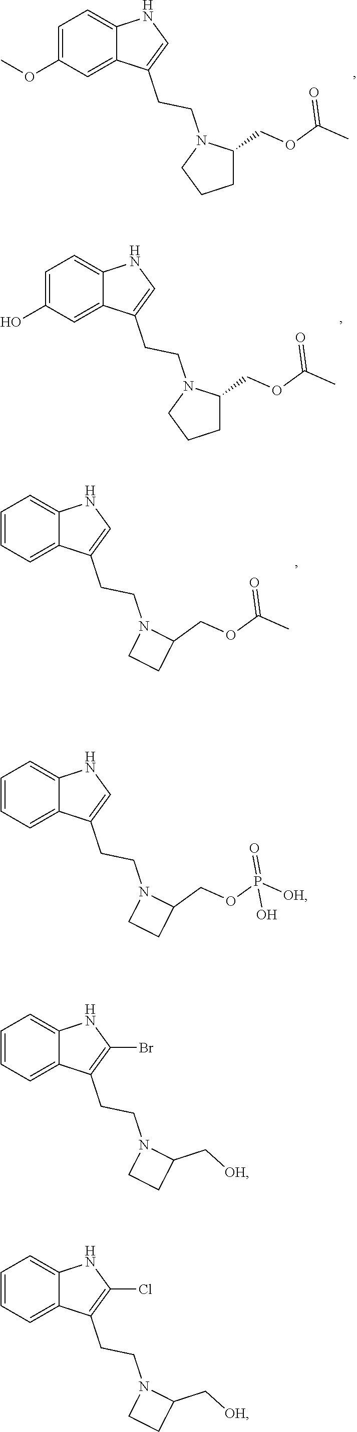 [SMILES: COc1ccc2[nH]cc(CCN3CCC[C@H]3COC(C)=O)c2c1 ; CC(=O)OC[C@@H]1CCCN1CCc1c[nH]c2ccc(O)cc12 ; CC(=O)OCC1CCN1CCc1c[nH]c2ccccc12 ; O=P(O)(O)OCC1CCN1CCc1c[nH]c2ccccc12 ; OCC1CCN1CCc1c(Br)[nH]c2ccccc12 ; OCC1CCN1CCc1c(Cl)[nH]c2ccccc12]

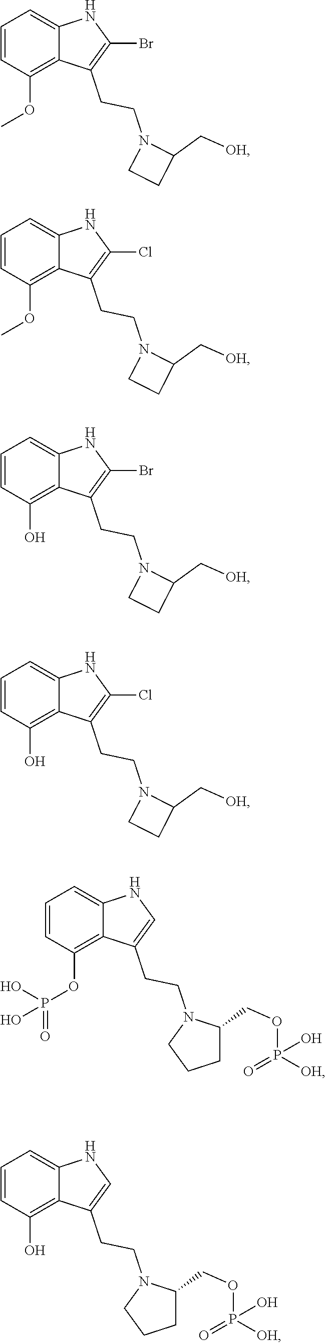 [SMILES: COc1cccc2[nH]c(Br)c(CCN3CCC3CO)c12 ; COc1cccc2[nH]c(Cl)c(CCN3CCC3CO)c12 ; OCC1CCN1CCc1c(Br)[nH]c2cccc(O)c12 ; OCC1CCN1CCc1c(Cl)[nH]c2cccc(O)c12 ; O=P(O)(O)OC[C@@H]1CCCN1CCc1c[nH]c2cccc(OP(=O)(O)O)c12 ; O=P(O)(O)OC[C@@H]1CCCN1CCc1c[nH]c2cccc(O)c12]

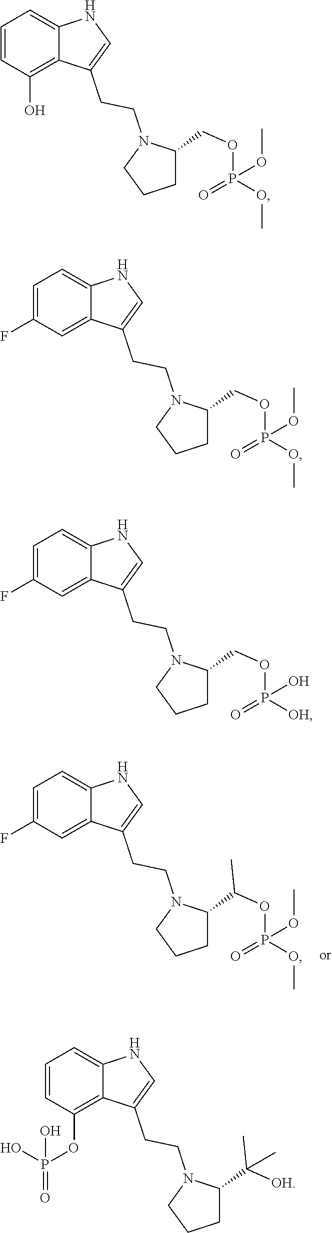 [SMILES: COP(=O)(OC)OC[C@@H]1CCCN1CCc1c[nH]c2cccc(O)c12 ; COP(=O)(OC)OC[C@@H]1CCCN1CCc1c[nH]c2ccc(F)cc12 ; O=P(O)(O)OC[C@@H]1CCCN1CCc1c[nH]c2ccc(F)cc12 ; COP(=O)(OC)OC(C)[C@@H]1CCCN1CCc1c[nH]c2ccc(F)cc12 ; "or" ; CC(C)(O)[C@@H]1CCCN1CCc1c[nH]c2cccc(OP(=O)(O)O)c12]

53. A pharmaceutical composition, comprising a compound of any one of embodiments 1-52 and a pharmaceutically acceptable excipient.
54. A method of treating a mental health disease or disorder, the method comprising administering a therapeutically effective amount of a compound of any one of embodiments 1-52 or pharmaceutical composition of embodiment 53.

### Examples

Synthesis of Compounds of the Disclosure:

Experimental Details:

Synthesis of Common Intermediate Aldehyde A

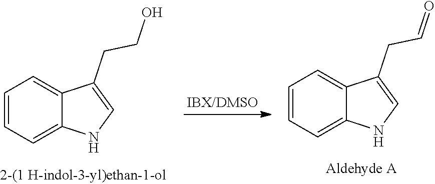 [SMILES: OCCc1c[nH]c2ccccc12 ; O=CCc1c[nH]c2ccccc12]

To a solution of 2-(1H-indol-3-yl)ethan-1-ol (6 g, 37.27 mmol, 1.0 equiv) in dimethyl sulfoxide (60 mL) was added 2-Iodoxybenzoic acid (12.5 g, 44.7 mmol, 1.2 equiv) at 40° C. under nitrogen. The mixture as stirred for 2 h at 40° C. The solution was diluted with 200 mL of dichloromethane, washed with 2×200 mL saturated sodium bicarbonate solution and 2×200 mL saturated aqueous sodium chloride respectively. The organic layer was dried over anhydrous sodium sulfate, filtered and concentrated under vacuum. The residue was applied onto a silica gel column with petroleum ether/ethyl acetate (3:1). 4 gram of 2-(1H-indol-3-yl)acetaldehyde the desired product was obtained.

The 2-(1H-indol-3-yl)acetaldehyde (A) was used for the synthesis for all compounds in Table 1.

### Synthesis of Compound 1: methyl (2-(1H-indol-3-yl)ethyl)prolinate

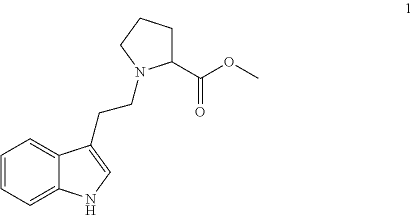 [SMILES: COC(=O)C1CCCN1CCc1c[nH]c2ccccc12]

To a solution of aldehyde A (100 mg, 0.63 mmol, 1.00 equiv) and H-DL-pro-ome HCL (311.8 mg, 1.89 mmol, 3.00 equiv) in dichloromethane (1 mL) were added triethylamine (190.9 mg, 1.89 mmol, 3.00 equiv) and stannic chloride pentahydrate (112 mg, 0.32 mmol, 0.5 equiv) at 25° C. The resulting mixture was stirred for 1 h at 25° C. under nitrogen atmosphere. To the above mixture was added sodium cyanoborohydride (58.6 mg, 0.95 mmol, 1.50 equiv) in portions. The mixture was stirred for 2 h at 25° C. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. methyl (2-(1H-indol-3-yl)ethyl)prolinate (25.8 mg) was obtained. MS m/z [M+H]⁺ (ESI): 273.10. ¹H NMR (300 MHz, DMSO-d6) δ10.77 (s, 1H), 7.49-7.46 (m, 1H), 7.34-7.31 (m, 1H), 7.16 (d, J=2.4 Hz, 1H), 7.08-7.02 (m, 1H), 6.99-6.94 (m, 1H), 3.61 (s, 3H), 3.31-3.23 (m, 1H), 3.14-3.04 (m, 1H), 3.00-2.87 (m, 1H), 2.87-2.74 (m, 2H), 2.70-2.60 (m, 1H), 2.49-2.42 (m, 1H), 2.10-1.95 (m, 1H), 1.88-1.71 (m, 3H).

### Synthesis of Compound 2: (2-(1H-indol-3-yl)ethyl)proline

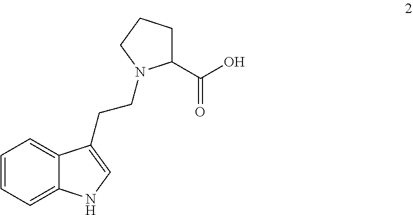 [SMILES: O=C(O)C1CCCN1CCc1c[nH]c2ccccc12]

To a solution of aldehyde A (100 mg, 0.63 mmol, 1.00 equiv) and DL-proline (217.3 mg, 1.89 mmol, 3.00 equiv) in dichloromethane (1 mL) were added triethylamine (190.9 mg, 1.89 mmol, 3.00 equiv) and stannic chloride pentahydrate (112 mg, 0.32 mmol, 0.5 equiv) at 25° C. The resulting mixture was stirred for 1 h at 25° C. under nitrogen atmosphere. To the above mixture was added sodium cyanoborohydride (58.6 mg, 0.95 mmol, 1.50 equiv) in portions. The mixture was stirred for 2 h at 25° C. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. 3.5 mg (2-(1H-indol-3-yl)ethyl)proline was obtained. MS m/z [M+H]⁺ (ESI): 259.05. ¹H NMR (300 MHz, DMSO-d6) δ 10.90 (s, 1H), 7.58-5.55 (m, 1H), 7.36-7.32 (m, 1H), 7.22-6.96 (m, 3H), 3.64-3.55 (m, 3H), 3.20-3.14 (m, 1H), 3.04-2.91 (m, 3H), 2.26-2.09 (m, 1H), 1.99-1.85 (m, 2H), 1.76-1.63 (m, 1H).

### Synthesis of Compound 3: (1-(2-(1H-indol-3-yl)ethyl)pyrrolidin-2-yl)methanol

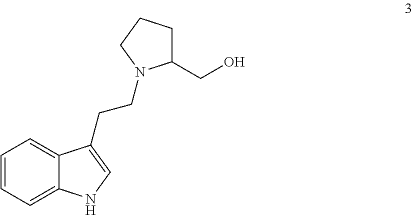 [SMILES: OCC1CCCN1CCc1c[nH]c2ccccc12]

To a solution of aldehyde A (100 mg, 0.63 mmol, 1.00 equiv) and 2-(Hydroxymethyl)pyrrolidine (190.9 mg, 1.89 mmol, 3.00 equiv) in dichloromethane (1 mL) were added triethylamine (190.9 mg, 1.89 mmol, 3.00 equiv) and stannic chloride pentahydrate (112 mg, 0.32 mmol, 0.5 equiv) at 25° C. The resulting mixture was stirred for 1 h at 25° C. under nitrogen atmosphere. To the above mixture was added sodium cyanoborohydride (58.6 mg, 0.95 mmol, 1.50 equiv) in portions. The mixture was stirred for 2 h at 25° C. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. 29.5 mg of (1-(2-(1H-indol-3-yl)ethyl)pyrrolidin-2-yl)methanol was obtained. MS m/z [M+H]⁺ (ESI): 245.20. ¹H NMR (300 MHz, DMSO-d6) δ 10.74 (s, 1H), 7.51-7.48 (m, 1H), 7.33-7.31 (m, 1H), 7.14-6.92 (m, 3H), 3.43-3.38 (m, 2H), 3.26-3.00 (m, 4H), 2.90-2.73 (m, 2H), 2.30-2.20 (m, 1H), 1.84-1.74 (m, 1H), 1.67-1.53 (m, 3H).

### Synthesis of Compound 4: methyl N-(2-(1H-indol-3-yl)ethyl)-N-methylglycinate

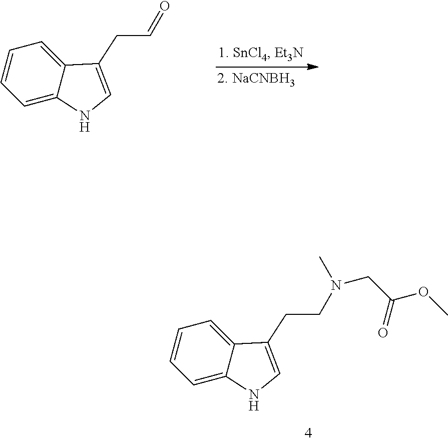 [SMILES: O=CCc1c[nH]c2ccccc12 ; COC(=O)CN(C)CCc1c[nH]c2ccccc12]

To a solution of 2-(1H-indol-3-yl)acetaldehyde (A) (100 mg, 0.63 mmol, 1.00 equiv) and 263 mg of sarcosine methyl ester hydrochloride in dichloromethane (1 mL) were added triethylamine (191 mg, 1.89 mmol, 3.00 equiv) and the solution of SnCl₄ in dichloromethane (1N, 0.32 mL, 0.5 equiv) at 25° C. The resulting mixture was stirred for 1 h at 25° C. under nitrogen. Sodium cyanoborohydride (59 mg, 0.95 mmol, 1.50 equiv) was added in portions. The mixture was stirred for 2 h at 25° C. The reaction was diluted with 20 mL of dichloromethane. The organic layer was washed with 2×20 mL saturated aqueous sodium chloride solution, dried over anhydrous sodium sulfate, filtered and concentrated under vacuum. The crude product was purified by Preparative-HPLC twice using the following conditions: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 22% B to 50% B in 9 min, 50% B; Wavelength: 254 nm. 22 mg of compound methyl N-(2-(1H-indol-3-yl)ethyl)-N-methylglycinate (4) was obtained. MS m/z [M+H]⁺ (ESI): 247.05. ¹H NMR (300 MHz, DMSO-d6) δ 10.76 (s, 1H), 7.50 (d, J=7.8 Hz, 1H), 7.32 (d, J=7.8 Hz, 1H), 7.14 (d, J=2.1 Hz, 1H), 7.08-7.02 (m, 1H), 6.99-6.93 (m, 1H), 3.61 (s, 3H), 3.32 (s, 2H), 2.86-2.79 (m, 2H), 2.77-2.71 (m, 2H), 2.36 (s, 3H).

### Synthesis of Compound 5: N-(2-(1H-indol-3-yl)ethyl)-N-methylglycine

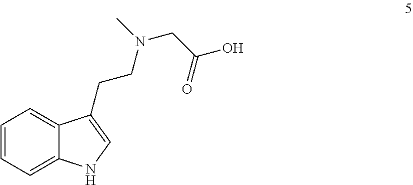 [SMILES: CN(CCc1c[nH]c2ccccc12)CC(=O)O]

N-(2-(1H-indol-3-yl)ethyl)-N-methylglycine (13.8 mg) was prepared using the same as for the preparation of 4. MS m/z [M+H]⁺ (ESI): 233.05. ¹H NMR (300 MHz, DMSO-d6) δ 10.85 (s, 1H), 7.57-7.52 (m, 1H), 7.35-7.31 (m, 1H), 7.17-6.93 (m, 3H), 3.21 (s, 2H), 2.95 (s, 4H), 2.55 (s, 3H).

### Synthesis of Compound 6: 2-((2-(1H-indol-3-yl)ethyl)(methyl)amino)ethan-1-ol

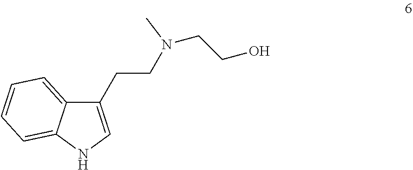 [SMILES: CN(CCO)CCc1c[nH]c2ccccc12]

2-((2-(1H-indol-3-yl)ethyl)(methyl)amino)ethan-1-ol (6) was prepared using the same as for the preparation of 4. 34 mg compound 6 was obtained. MS m/z [M+H]⁺ (ESI): 219.05. ¹H NMR (300 MHz, DMSO-d6) δ 10.68 (s, 1H), 7.42 (d, J=7.8 Hz, 1H), 7.25 (d, J=7.8 Hz, 1H), 7.07 (d, J=2.1 Hz, 1H), 7.00-6.87 (m, 2H), 4.25 (t, J=5.4 Hz, 1H), 3.45-3.39 (m, 2H), 2.77-2.72 (m, 2H), 2.66-2.54 (m, 2H), 2.44-2.40 (m, 2H), 2.21 (s, 3H).

### Synthesis of Compound 7: 1-(2-(1H-indol-3-yl)ethyl)pyrrolidin-3-ol

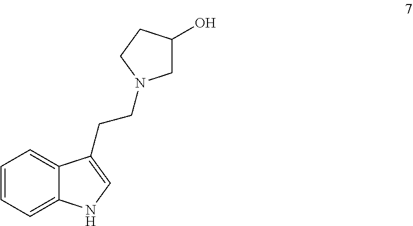 [SMILES: OC1CCN(CCc2c[nH]c3ccccc23)C1]

In a similar manner described for 4, compound 1-(2-(1H-indol-3-yl)ethyl)pyrrolidin-3-ol (5.5 mg) was prepared. MS m/z [M+H]⁺ (ESI): 231.05. ¹H NMR (300 MHz, DMSO-d6) δ 10.75 (s, 1H), 7.49 (d, J=7.8 Hz, 1H), 7.32 (d, J=7.8 Hz, 1H), 7.13 (s, 1H), 7.07-7.02 (m, 1H), 6.98-6.93 (m, 1H), 4.67 (s, 1H), 4.19 (s, 1H), 2.84-2.73 (m, 3H), 2.67-2.58 (m, 3H), 2.45-2.34 (m, 2H), 2.01-1.95 (m, 1H), 1.56-1.51 (m, 1H).

### Synthesis of Compound 8: 1-(2-(1H-indol-3-yl)ethyl)pyrrolidine-2-carboxamide

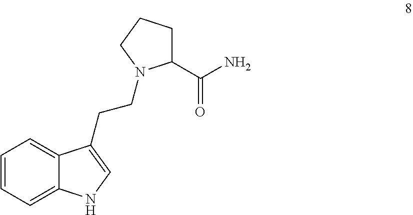 [SMILES: NC(=O)C1CCCN1CCc1c[nH]c2ccccc12]

To a solution of pyrrplodine-2-carboxamide (215.5 mg, 1.89 mmol, 3.00 equiv) and acetic acid (0.1 mL) in methanol (1 mL) were added sodium cyanoborohydride (58.6 mg, 0.95 mmol, 1.50 equiv) at 0° C. To the above mixture was added Aldehyde A (100 mg, 0.63 mmol, 1.00 equiv) in portions. The mixture was stirred for 2 h at 0° C. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. 40.9 mg 1-(2-(1H-indol-3-yl)ethyl)pyrrolidine-2-carboxamide, Compound 8 was obtained. MS m/z [M+H]⁺ (ESI): 258.10. ¹H NMR (400 MHz, DMSO-d6) δ 10.79 (s, 1H), 7.50 (d, J=8.0 Hz, 1H), 7.32 (d, J=8.0 Hz, 1H), 7.15 (d, J=2.4 Hz, 1H), 7.07-6.94 (m, 4H), 3.28-3.24 (m, 1H), 2.92-2.82 (m, 4H), 2.67-2.58 (m, 1H), 2.39-2.32 (m, 1H), 2.08-1.99 (m, 1H), 1.80-1.64 (m, 3H).

### Synthesis of Compound 9: (1-(2-(1H-indol-3-yl)ethyl)pyrrolidin-3-yl)methanol

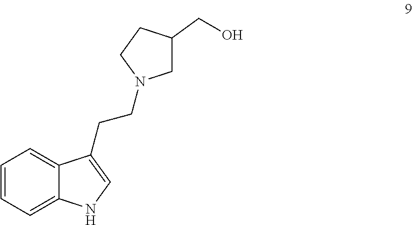 [SMILES: OCC1CCN(CCc2c[nH]c3ccccc23)C1]

To a solution of aldehyde A (100 mg, 0.63 mmol, 1.00 equiv) and 3-Hydroxymethylpyrrolidine (190.9 mg, 1.89 mmol, 3.00 equiv) in dichloromethane (1 mL) were added triethylamine (190.9 mg, 1.89 mmol, 3.00 equiv) and stannic chloride pentahydrate (112 mg, 0.32 mmol, 0.5 equiv) at 25° C. The resulting mixture was stirred for 1 h at 25° C. under nitrogen atmosphere. To the above mixture was added sodium cyanoborohydride (58.6 mg, 0.95 mmol, 1.50 equiv) in portions. The mixture was stirred for 2 h at 25° C. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. 10.3 mg of (1-(2-(1H-indol-3-yl)ethyl)pyrrolidin-3-yl)methanol, Compound 9 was obtained. MS m/z [M+H]⁺ (ESI): 245.10. ¹H NMR (300 MHz, DMSO-d6) δ 10.75 (s, 1H), 7.51-7.46 (m, 1H), 7.34-7.30 (m, 1H), 7.16-6.96 (m, 3H), 4.77-4.23 (m, 1H), 3.55-3.35 (m, 2H), 2.86-2.81 (m, 2H), 2.69-2.58 (m, 3H), 2.57-2.54 (m, 2H), 2.37-2.33 (m, 1H), 2.25-2.16 (m, 1H), 1.86-1.73 (m, 1H), 1.45-1.29 (m, 1H).

### Synthesis of Compound 10: 1-(2-(1H-indol-3-yl)ethyl)-N-methylpyrrolidine-2-carboxamide

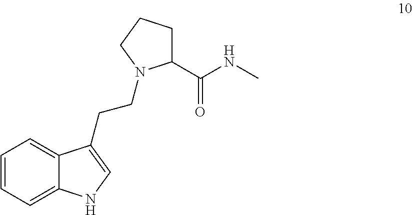 [SMILES: CNC(=O)C1CCCN1CCc1c[nH]c2ccccc12]

To a solution of Compound 2 (50 mg, 0.19 mmol, 1.00 equiv) and N,N-Diisopropylethylamine (73.5 mg, 0.57 mmol, 3.00 equiv) in dichloromethane (1 mL) was added HATU (216.6 mg, 0.57 mmol, 3.00 equiv) and methylamine hydrochloride (19.0 mg, 0.29 mmol, 1.5 equiv) at 25° C. The resulting mixture was stirred for 4 h at 25° C. under nitrogen atmosphere. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. 6.7 mg of 1-(2-(1H-indol-3-yl)ethyl)-N-methylpyrrolidine-2-carboxamide Compound 10. MS m/z [M+H]⁻ (ESI): 270.10. ¹H NMR (400 MHz, DMSO-d6) δ 10.82 (s, 1H), 7.50 (d, J=7.6 Hz, 1H), 7.35 (d, J=8.0 Hz, 1H), 7.25-7.22 (m, 1H), 7.17 (d, J=2.0 Hz, 1H), 7.09-7.04 (m, 1H), 7.01-6.96 (m, 1H), 3.30-3.26 (m, 1H), 2.94-2.78 (m, 4H), 2.71-2.63 (m, 1H), 2.38-2.28 (m, 4H), 2.07-1.97 (m, 1H), 1.77-1.56 (m, 3H).

### Synthesis of Compound 29: 3-(2-(2-(hydroxymethyl)pyrrolidin-1-yl)ethyl)-1H-indol-4-ol

Desired compound 29 was prepared in 4 steps from commercially available 4-methoxy-1H-indole, Scheme 1.

To a stirred solution of 4-methoxy-1H-indole, 29.1 (200 mg, 1.36 mmol, 1.0 equiv) in diethyl ether (5.0 mL) was added oxalyl chloride (0.18 g, 1.36 mmol, 1.0 equiv) dropwise at 0° C. under nitrogen atmosphere. The resulting mixture was stirred for additional 3h at room temperature. The resulting mixture 29.2 was used in the next step directly without further purification.

To a solution of 29.2 (100 mg, 0.42 mmol, 1.00 equiv) in tetrahydrofuran (2 mL) was added 2-(Hydroxymethyl)pyrrolidine (212.1 mg, 2.10 mmol, 5.00 equiv) and triethylamine (212.1 mg, 2.10 mmol, 5.00 equiv) at 0° C. The resulting mixture was stirred for 1 h at 25° C. under nitrogen atmosphere. The reaction was diluted with 20 mL of ethyl acetate. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. 100 mg of 29.3 was obtained. MS m/z [M+H]⁺ (ESI): 303.20.

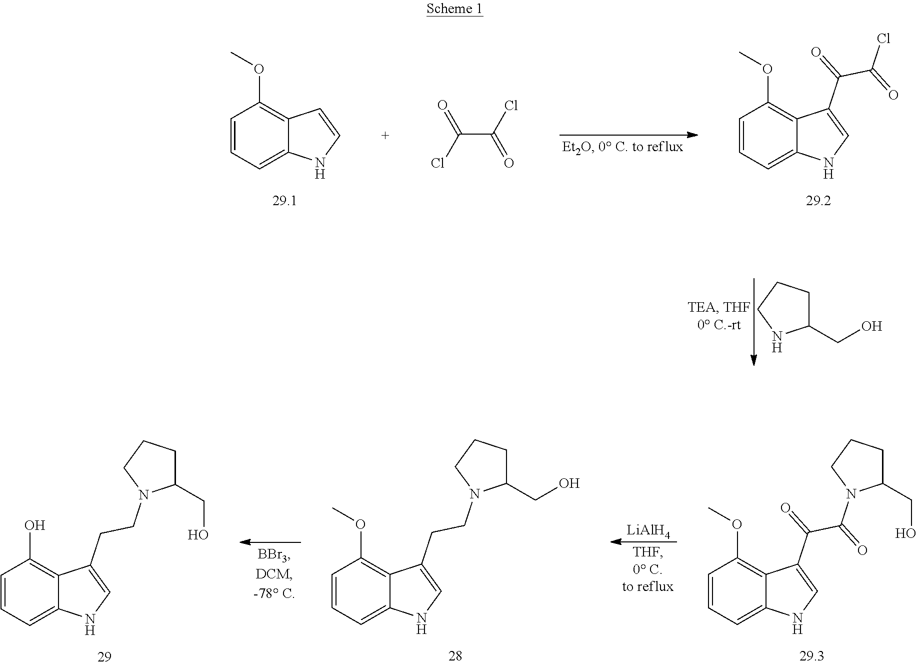

To a solution of 29.3 (100 mg, 0.33 mmol, 1.00 equiv) in tetrahydrofuran (2 mL) were added lithium aluminum hydride (31.5 mg, 0.83 mmol, 2.50 equiv) at 0° C. for 10 min. Then the reaction was stirred for 16 h at 66° C. The reaction was diluted with 20 mL of ethyl acetate. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. 50 mg of 28 was obtained. MS m/z [M+H]⁺ (ESI): 275.10.

To a solution of 29.4 (50 mg, 0.18 mmol, 1.00 equiv) and in dichloromethane (1 mL) were added boron tribromide (228.2 mg, 0.90 mmol, 5.0 equiv) at −78° C. The resulting mixture was stirred for 1 h at −78° C. The reaction was quenched with 1 mL of methanol at −78° C. and diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. 5.7 mg of Compound 29, 3-(2-(2-(hydroxymethyl)pyrrolidin-1-yl)ethyl)-1H-indol-4-ol: was obtained. MS m/z [M+H]⁺ (ESI): 261.05. ¹H NMR (400 MHz, Methanol-d4) δ6.93-6.84 (m, 3H), 6.38-6.36 (m, 1H), 3.64-3.55 (m, 2H), 3.45-3.35 (m, 2H), 3.14-3.10 (m, 2H), 3.02-2.95 (m, 2H), 2.75-2.68 (m, 1H), 2.14-2.05 (m, 1H), 1.96-1.88 (m, 2H), 1.82-1.74 (m, 1H).

### Synthesis of Compound 30: 3-(2-(2-(hydroxymethyl)pyrrolidin-1-yl)ethyl)-1H-indol-4-ol

Desired compound 30 was prepared in 2 steps from commercially available 2-(5-methoxy-1H-indol-3-yl)ethan-1-ol, Scheme 2.

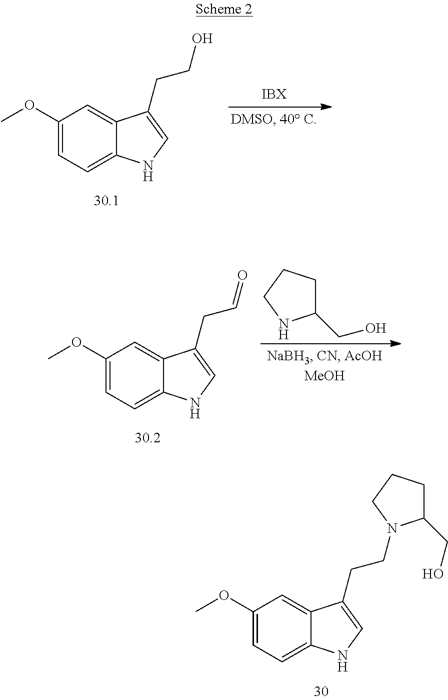

To a solution of 5-methoxytryptophol, 30.1 (200 mg, 1.05 mmol, 1.0 equiv) in dimethyl sulfoxide (2 mL) was added 2-Iodoxybenzoic acid (351.8 mg, 1.25 mmol, 1.2 equiv) at 40° C. under nitrogen atmosphere. The mixture solution was stirred for 2 h at 40° C. The reacting solution was diluted with 20 mL of dichloromethane, washed with 2×20 mL saturated sodium bicarbonate solution and 2×20 mL saturated aqueous sodium chloride respectively. The organic layer was dried over anhydrous sodium sulfate, filtered and concentrated under reduced pressure. The residue was applied onto a silica gel column with petroleum ether/ethyl acetate (3:1). 100 mg of 30.2 was obtained.

To a solution of 2-(Hydroxymethyl)pyrrolidine, (160.6 mg, 1.59 mmol, 3.00 equiv) and acetic acid (0.1 mL) in methanol (1 mL) was added sodium cyanoborohydride (50.4 mg, 0.80 mmol, 1.50 equiv) at 0° C. To the above mixture was added 30.2 (100 mg, 0.53 mmol, 1.00 equiv) in portions. The mixture was stirred for 2 h at 0° C. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃), 10% to 100% gradient in 20 min; detector, UV 254 nm. 23.5 mg of Compound 30, 3-(2-(2-(hydroxymethyl)pyrrolidin-1-yl)ethyl)-1H-indol-4-ol, was obtained as a brown. MS m/z [M+H]⁺ (ESI): 275.15. ¹H NMR (400 MHz, DMSO-d6) δ 10.59 (s, 1H), 7.21 (d, J=8.8 Hz, 1H), 7.10-6.97 (m, 2H), 6.72-6.67 (m, 1H), 4.34 (s, 1H), 3.76 (s, 3H), 3.26-3.03 (m, 6H), 2.86-2.70 (m, 2H), 2.28-2.21 (m, 1H), 1.85-1.76 (m, 1H), 1.70-1.50 (m, 3H).

### Synthesis of Compound 31: (S)-(1-(2-(1H-indol-3-yl)ethyl)pyrrolidin-2-yl)methanol

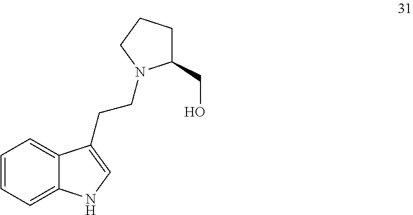 [SMILES: OC[C@@H]1CCCN1CCc1c[nH]c2ccccc12]

To a solution of L-(+)-Prolinol (954.4 mg, 9.45 mmol, 3.00 equiv) and acetic acid (0.5 mL) in methanol (5 mL) were added sodium cyanoborohydride (293 mg, 4.75 mmol, 1.50 equiv) at 0° C. To the above mixture was added aldehyde A (500 mg, 3.15 mmol, 1.00 equiv) in portions over 2 h at 0° C. The reaction was diluted with 50 mL of dichloromethane. The organic layer was washed with 2×50 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% HCl) and acetonitrile, 10% to 100% gradient in 20 min; detector, UV 254 nm. 237.4 mg of 31 was obtained. MS m/z [M+H]⁺ (ESI): 245.05. ¹H NMR (400 MHz, Methanol-d4) δ 7.65-7.60 (m, 1H), 7.42-7.37 (m, 1H), 7.22 (s, 1H), 7.17-7.11 (m, 1H), 7.10-7.03 (m, 1H), 3.94-3.88 (m, 1H), 3.83-3.72 (m, 3H), 3.71-3.62 (m, 1H), 3.41-3.34 (m, 1H), 3.30-3.21 (m, 3H), 2.26-1.98 (m, 3H), 1.94-1.82 (m, 1H).

### Synthesis of Compound 32: (R)-(1-(2-(1H-indol-3-yl)ethyl)pyrrolidin-2-yl)methanol

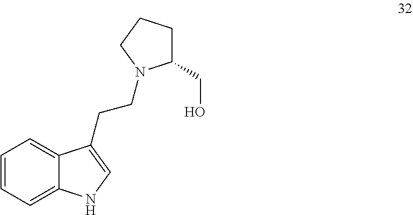 [SMILES: OC[C@H]1CCCN1CCc1c[nH]c2ccccc12]

To a solution of D-Prolinol (190.9 mg, 1.89 mmol, 3.00 equiv) and acetic acid (0.1 mL) in methanol (1 mL) were added sodium cyanoborohydride (58.6 mg, 0.95 mmol, 1.50 equiv) at 0° C. To the above mixture was added aldehyde A (100 mg, 0.63 mmol, 1.00 equiv) in portions over 2 h at 0° C. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃) and acetonitrile, 10% to 100% gradient in 20 min; detector, UV 254 nm. 20.7 mg of 32, (S)-(1-(2-(1H-indol-3-yl)ethyl)pyrrolidin-2-yl)methanol was obtained. MS m/z [M+H]⁺ (ESI): 245.15. ¹H NMR (400 MHz, DMSO-d6) δ 10.76 (s, 1H), 7.51 (d, J=7.8 Hz, 1H), 7.33 (d, J=8.0 Hz, 1H), 7.15 (d, J=2.3 Hz, 1H), 7.10-7.02 (m, 1H), 7.00-6.94 (m, 1H), 4.52-3.90 (m, 1H), 3.52-3.39 (m, 1H), 3.27-3.20 (m, 1H), 3.19-3.13 (m, 1H), 3.12-3.03 (m, 1H), 2.92-2.75 (m, 2H), 2.58-2.53 (m, 1H), 2.49-2.44 (m, 1H), 2.34-2.18 (m, 1H), 1.87-1.75 (m, 1H), 1.73-1.51 (m, 3H).

### Synthesis of Compound 33: methyl (2-(1H-indol-3-yl)ethyl)-L-prolinate

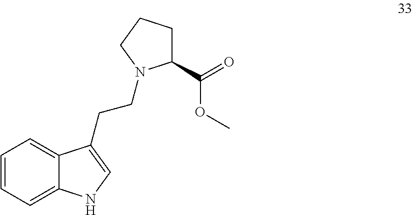 [SMILES: COC(=O)[C@@H]1CCCN1CCc1c[nH]c2ccccc12]

To a stirred solution/mixture of methyl (2S)-pyrrolidine-2-carboxylate hydrochloride (2.1 g, 12.56 mmol, 10.0 equiv), sodium cyanoborohydride (394.8 mg, 6.28 mmol, 5.0 equiv) and Acetic acid (0.2 mL) in methyl alcohol (2.0 mL) were added Aldehyde A (200.0 mg, 1.26 mmol, 1.0 equiv) dropwise/in portions at 0° C. under nitrogen atmosphere. The aqueous layer was extracted with ethyl acetate (3×30.0 mL). The residue was purified by reversed-phase flash chromatography with the following conditions: column, C18 silica gel; mobile phase, acetonitrile in Water (10 mmol/L NH₄HCO₃), 10% to 100% gradient in 20 min; detector, UV 254 nm. to afford 40.2 mg of compound 33, methyl (2-(1H-indol-3-yl)ethyl)-L-prolinate. MS m/z [M+H]⁺ (ESI): 273.20. H NMR (300 MHz, DMSO-d6) δ 10.77 (s, 1H), 7.48 (d, J=8.2 Hz, 1H), 7.35-7.31 (m, 1H), 7.16 (d, J=2.4 Hz, 1H), 7.08-6.94 (m, 2H), 3.61 (s, 3H), 3.29-3.25 (m, 1H), 3.13-3.06 (m, 1H), 2.99-2.76 (m, 3H), 2.69-2.61 (m, 1H), 2.50-2.43 (m, 1H), 2.11-1.98 (m, 1H), 1.89-1.73 (m, 3H).

### Synthesis of Compound 34: methyl (2-(1H-indol-3-yl)ethyl)-D-prolinate

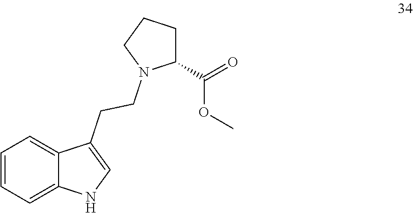 [SMILES: COC(=O)[C@H]1CCCN1CCc1c[nH]c2ccccc12]

To a stirred solution/mixture of methyl (2R)-pyrrolidine-2-carboxylate hydrochloride (2.1 g, 12.56 mmol, 10.0 equiv), sodium cyanoborohydride (394.8 mg, 6.28 mmol, 5.0 equiv) and Acetic acid (0.2 mL) in methyl alcohol (2.0 mL) were added aldehyde (200.0 mg, 1.26 mmol, 1.0 equiv) dropwise in portions at 0° C. under nitrogen atmosphere. The aqueous layer was extracted with ethyl acetate (3×30.0 mL). The residue was purified by reversed-phase flash chromatography with the following conditions: column, C18 silica gel; mobile phase, acetonitrile in Water (10 mmol/L NH₄HCO₃), 10% to 100% gradient in 100 min; detector, UV 254 nm. to afford c26.5 mg of compound 34, methyl (2-(1H-indol-3-yl)ethyl)-D-prolinate. MS m/z [M+H]⁺ (ESI): 273.10. H NMR (300 MHz, DMSO-d6) δ 10.77 (s, 1H), 7.48 (d, J=7.8 Hz, 1H), 7.34-7.31 (m, 1H), 7.16 (d, J=2.3 Hz, 1H), 7.03-6.94 (m, 2H), 3.61 (s, 3H), 3.33-3.25 (m, 1H), 3.15-3.05 (m, 1H), 3.00-2.89 (m, 1H), 2.89-2.83 (m, 1H), 2.82-2.74 (m, 1H), 2.71-2.60 (m, 1H), 2.49-2.43 (m, 1H), 2.12-1.97 (m, 1H), 1.88-1.73 (m, 3H).

### Synthesis of Compound 35: 3-(2-(2-(hydroxymethyl)pyrrolidin-1-yl)ethyl)-1H-indol-4-ol

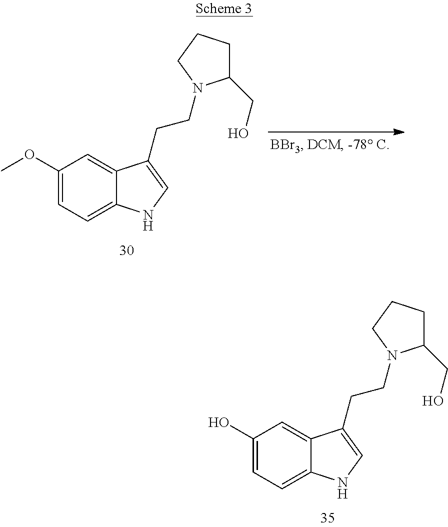 [SMILES: COc1ccc2[nH]cc(CCN3CCCC3CO)c2c1 ; OCC1CCCN1CCc1c[nH]c2ccc(O)cc12]

To a solution of 30 (100 mg, 0.36 mmol, 1.00 equiv) in dichloromethane (1 mL) was added boron tribromide (456.2 mg, 1.82 mmol, 5.0 equiv) at −78° C. The mixture was stirred for 1 h at -78° C. The reaction was quenched with 1 mL of methanol at −78° C. and diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% FA), 10% to 100% gradient in 20 min; detector, UV 254 nm. 32.2 mg of compound 35, 3-(2-(2-(hydroxymethyl)pyrrolidin-1-yl)ethyl)-1H-indol-4-ol was obtained. MS m/z [M+H]⁺ (ESI): 261.05. ¹H NMR (400 MHz, Methanol-d4) δ 7.21 (d, J=8.4 Hz, 1H), 7.15 (s, 1H), 6.95 (d, J=2.0 Hz, 1H), 6.74-6.71 (m, 1H), 3.94-3.90 (m, 1H), 3.81-3.63 (m, 4H), 3.39-3.36 (m, 1H), 3.30-3.25 (m, 1H), 3.20-3.16 (m, 2H), 2.29-2.11 (m, 2H), 2.09-1.99 (m, 1H), 1.96-1.88 (m, 1H).

### Synthesis of Compound 42: (1-(2-(7-bromo-4-methoxy-1H-indol-3-yl)ethyl)pyrrolidin-2-yl)methanol

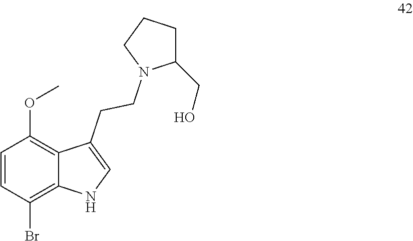 [SMILES: COc1ccc(Br)c2[nH]cc(CCN3CCCC3CO)c12]

To a solution of 28 (300 mg, 1.093 mmol, 1 equiv) in 1,2-dichloroethane (3.00 mL, 34.66 equiv) was added the solution of dibromocopper (0.73 g, 3.279 mmol, 3 equiv) in acetonitrile (3 mL) by dropwise at 0° C. Then, the mixture was stirred for one hour at room temperature. The residue was purified by reversed-phase flash chromatography with the following conditions: column, C18 silica gel; mobile phase, acetonitrile in water (0.1% NH₃·H₂O+10 mmol/L NH₄HCO₃), 5% to 50% gradient in 12 min; detector, UV 254 nm. This resulted in a crude product. The residue was purified by column: XBridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient: 35% B to 55% B in 12 min; Wave Length: 220 nm; RT1(min): 7.52. This resulted in 19.1 mg of desired compound 42, (1-(2-(7-bromo-4-methoxy-1H-indol-3-yl)ethyl)pyrrolidin-2-yl)methanol. MS m/z [M+H]⁺ (ESI): 353.15, 355.15. ¹H NMR (300 MHz, Methanol-d₄) δ 7.13 (d, J=8.1 Hz, 1H), 7.00 (s, 1H), 6.42 (d, J=8.1 Hz, 1H), 3.91 (s, 3H), 3.69-3.61 (m, 1H), 3.58-3.50 (m, 1H), 3.40-3.33 (m, 1H), 3.29-3.18 (m, 1H), 3.15-2.97 (m, 2H), 2.86-2.63 (m, 2H), 2.63-2.50 (m, 1H), 2.10-1.96 (m, 1H), 1.91-1.79 (m, 2H), 1.78-1.64 (m, 1H).

### Synthesis of Compound 48 (S)-(1-(2-(4-methoxy-1H-indol-3-yl)ethyl)pyrrolidin-2-yl)methanol and 49 (S)-3-(2-(2-(hydroxymethyl)pyrrolidin-1-yl)ethyl)-1H-indol-4-ol

48 and 49 was prepared from previously synthesized intermediate 29.2. Described in Scheme 4.

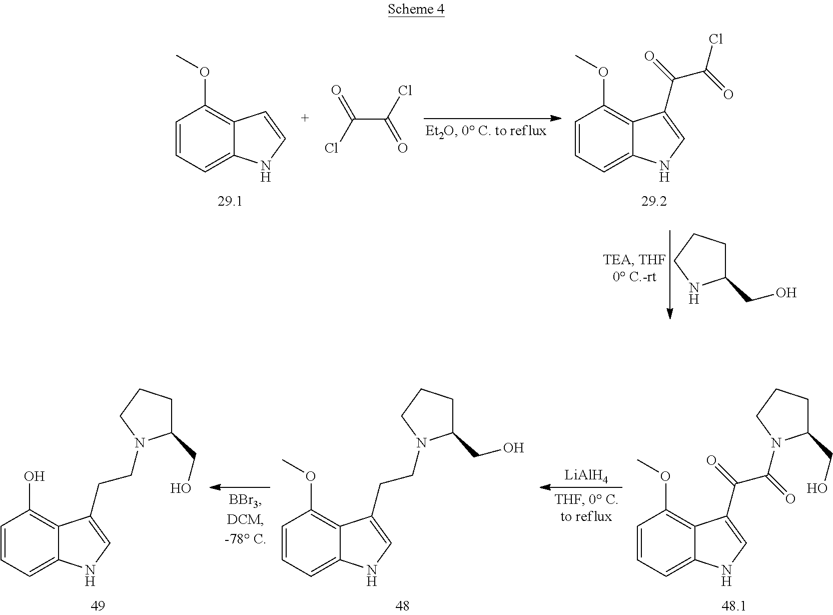

To a solution of 29.2 (200 mg, 0.84 mmol, 1.00 equiv) in tetrahydrofuran (4 mL) were added L-Prolinol (424.2 mg, 4.20 mmol, 5.00 equiv) and triethylamine (424.2 mg, 2.10 mmol, 5.00 equiv) at 0° C. The resulting mixture was stirred for 1 h at 25° C. under nitrogen atmosphere. The reaction was diluted with 20 mL of ethyl acetate. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃) and acetonitrile, 10% to 100% gradient in 20 min; detector, UV 254 nm. 200 mg of 48.1 was obtained. MS m/z [M+H]⁺ (ESI): 303.20.

To a solution of 48.1 (200 mg, 0.66 mmol, 1.00 equiv) in tetrahydrofuran (4 mL) were added lithium aluminum hydride (63.0 mg, 1.66 mmol, 2.50 equiv) at 0° C. for 10 min. Then the reaction was stirred for 16 h at 66° C. The reaction was diluted with 20 mL of ethyl acetate. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃) and acetonitrile, 10% to 100% gradient in 20 min; detector, UV 254 nm. 100 mg of 48, (S)-(1-(2-(4-methoxy-1H-indol-3-yl)ethyl)pyrrolidin-2-yl)methanol was obtained. MS m/z [M+H]⁺ (ESI): 275.15. ¹H NMR (400 MHz, Methanol-d4) δ 7.01 (t, J=7.9 Hz, 1H), 6.97-6.92 (m, 2H), 6.48 (d, J=7.6 Hz, 1H), 3.93 (s, 3H), 3.75-3.67 (m, 1H), 3.63-3.54 (m, 1H), 3.40 (s, 1H), 3.32-3.26 (m, 1H), 3.21-3.03 (m, 2H), 2.96-2.55 (m, 3H), 2.12-2.00 (m, 1H), 1.97-1.82 (m, 2H), 1.82-1.68 (m, 1H). HRMS m/z [M+H]⁺ (ESI): calc. Mass 275.1760; found 275.1764. ¹³CNMR (75 MHz, Methanol-d₄) δ 154.25, 138.55, 121.86. 120.81, 116.93, 111.98, 104.46, 98.30, 66.41, 62.68, 57.44, 54.01, 53.94, 27.23, 25.08, 22.23.

To a solution of 48 (100 mg, 0.36 mmol, 1.00 equiv) and in dichloromethane (2 mL) were added boron tribromide (556.4 mg, 1.80 mmol, 5.0 equiv) at −78° C. The resulting mixture was stirred for 1 h at −78° C. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃) and acetonitrile, 10% to 100% gradient in 20 min; detector, UV 254 nm. 16.6 mg of 49, (S)-3-(2-(2-(hydroxymethyl)pyrrolidin-1-yl)ethyl)-1H-indol-4-ol was obtained. MS m/z [M+H]⁺ (ESI): 261.05. ¹H NMR (400 MHz, Methanol-d4) δ 6.95-6.81 (m, 3H), 6.40-6.32 (m, 1H), 3.64-3.56 (m, 1H), 3.55-3.47 (m, 1H), 3.41-3.35 (m, 1H), 3.30-3.23 (m, 1H), 3.13-3.04 (m, 2H), 2.94-2.83 (m, 2H), 2.66-2.57 (m, 1H), 2.11-2.02 (m, 1H), 1.94-1.85 (m, 2H), 1.81-1.71 (m, 1H). HRMS m/z [M+H]⁺ (ESI): calc. Mass 261.1603; found 261.1599. ¹³CNMR (75 MHz, Methanol-d₄) δ 150.99, 139.25, 122.39, 121.48, 116.15, 108.97, 103.18, 102.70, 68.41, 58.91, 54.12, 48.45, 25.65, 23.13, 22.08.

### Synthesis of Compound 50: (S)-3-(2-(2-(hydroxymethyl)pyrrolidin-1-yl)ethyl)-1H-indol-5-ol and 51

(S)-(1-(2-(5-methoxy-1H-indol-3-yl)ethyl)pyrrolidin-2-yl)methanol. Described in Scheme 5.

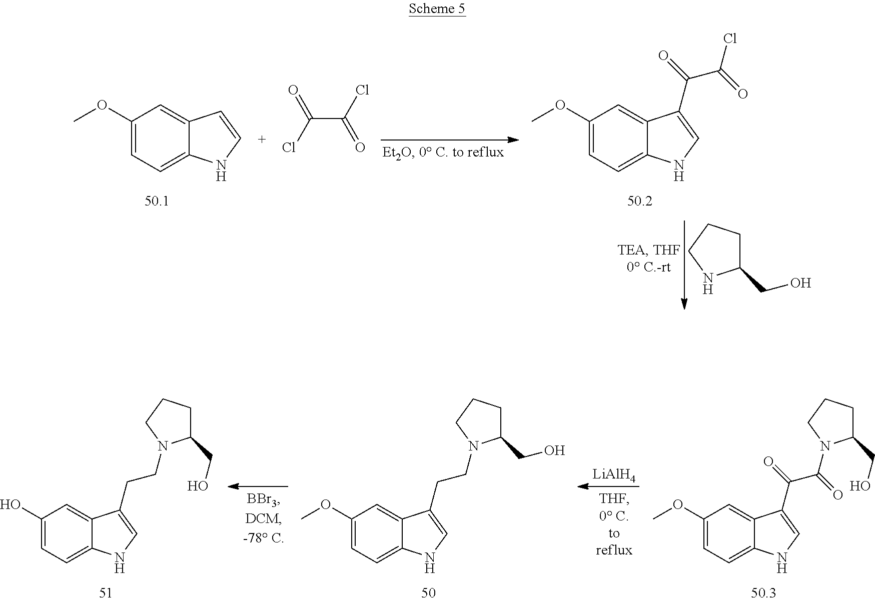

To a solution of 5-methoxyindole 50.1 (5 g, 33.973 mmol, 1.00 equiv) in diethyl ether (100 mL) was added oxalyl chloride (4.31 g, 33.973 mmol, 1.00 equiv) by dropwise at 0° C. The mixture was stirred for one hour. It was used at next step directly.

A solution of 50.2 (5 g, 21.040 mmol, 1 equiv) in tetrahydrofuran (100 mL) was treated with prolinol (10.64 g, 105.200 mmol, 5 equiv) for 30 min at 0° C. Into a 3-necked round-bottom flask were added C₆H₁₅N (21.29 g, 210.400 mmol, 10 equiv) at 0° C. The resulting mixture was stirred for 4 h at room temperature. The reaction was diluted with 20 mL of ethyl acetate. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (0.05% ammonium bicarbonate) and acetonitrile (20% acetonitrile up to 100% within 15 min, hold 100% for 5 min; Detector, UV 254 nm). 1.5 g of 50.3 was obtained. MS m/z [M+H]⁺ (ESI): 302.13. This crude material was used directly for the next step.

To a solution of 50.3 (500 mg, 1.654 mmol, 1.00 equiv) in tetrahydrofuran (10 mL) were added lithium aluminum hydride (156.92 mg, 4.134 mmol, 2.50 equiv) at 0° C. for 10 min. Then the reaction was stirred for 16 h at 66° C. The reaction was diluted with 20 mL of ethyl acetate. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (0.05% ammonium bicarbonate) and acetonitrile (20% acetonitrile up to 100% within 15 min, hold 100% for 5 min; Detector, UV 254 nm). 150 mg of compound 50, (S)-3-(2-(2-(hydroxymethyl)pyrrolidin-1-yl)ethyl)-1H-indol-5-ol was obtained. MS m/z [M+H]⁺ (ESI): 275.15. ¹H NMR (400 MHz, CD₃OD) δ 7.24 (d, J=8 Hz, 1H), 7.04-7.02 (m, 2H), 6.76-6.73 (m, 1H), 3.81 (s, 3H), 3.66-3.57 (m, 2H), 3.55-3.37 (m, 1H), 3.32-3.30 (m, 1H), 3.29-2.95 (m, 2H), 2.85-2.75 (m, 2H), 2.59-2.56 (m, 1H), 2.02-1.99 (m, 1H), 1.88-1.84 (m, 2H), 1.71 (s, 1H). HRMS m/z [M+H]⁺ (ESI): calc. Mass 275.1760; found 275.1772. ¹³CNMR (75 MHz, Methanol-d₄) δ 153.49, 132.00, 127.58. 122.43, 112.32, 111.49, 111.06, 99.94, 65.83, 63.73, 56.14, 54.92, 54.12, 27.51, 23.98, 22.30.

A solution of compound 50 (1.00 g, 3.64 mmol, 1.00 equiv) and BBr₃ (4.57 g, 18.22 mmol, 5.00 equiv) in DCM (10 mL) was stirred for 2 h at −78° C. The reaction was diluted with 20 mL of dichloromethane. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% ammonium bicarbonate), 10% to 100% gradient in 20 min; detector, UV 254 nm. 144.4 mg of compound 51, (S)-(1-(2-(5-methoxy-1H-indol-3-yl)ethyl)pyrrolidin-2-yl)methanol was obtained. MS m/z [M+H]⁺ (ESI): 261.25. ¹H NMR (400 MHz, Methanol-d₄) δ 7.13 (d, J=8.6 Hz, 1H), 7.02 (s, 1H), 6.93 (d, J=2.3 Hz, 1H), 6.71-6.64 (m, 1H), 3.70-3.62 (m, 1H), 3.59-3.50 (m, 1H), 3.40-3.34 (m, 1H), 3.30-3.18 (m, 1H), 3.02-2.92 (m, 1H), 2.95-2.85 (m, 1H), 2.77-2.63 (m, 2H), 2.56-2.45 (m, 1H), 2.02-1.97 (m, 1H), 1.85-1.80 (m, 2H), 1.73-1.68 (m, 1H). HRMS m/z [M+H]⁺ (ESI): calc. Mass 261.1603; found 261.1604. ¹³CNMR (75 MHz, Methanol-d₄) δ 149.69, 131.69, 127.93. 122.59, 111.50, 111.30, 110.94, 102.06, 66.09, 63.42, 56.12, 54.11, 27.42, 23.89, 22.26.

### Synthesis of Compound 50: ethyl (2-(1H-indol-3-yl)ethyl)-L-prolinate

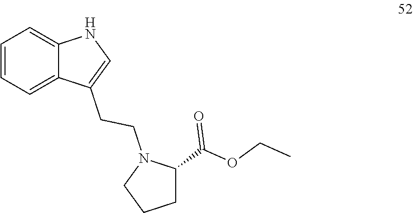 [SMILES: CCOC(=O)[C@@H]1CCCN1CCc1c[nH]c2ccccc12]

To a solution of L-Proline ethyl ester hydrochloride (338.3 mg, 1.89 mmol, 3.00 equiv) and acetic acid (0.1 mL) in methanol (1 mL) were added sodium cyanoborohydride (58.6 mg, 0.95 mmol, 1.50 equiv) at 0° C. To the above mixture was added Aldehyde A (100 mg, 0.63 mmol, 1.00 equiv) in portions over 2 h at 0° C. The reaction was diluted with 10 mL of dichloromethane. The organic layer was washed with 2×10 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% NH₄HCO₃) and acetonitrile, 10% to 100% gradient in 20 min; detector, UV 254 nm. 31.4 mg of Compound 52, ethyl (2-(1H-indol-3-yl)ethyl)-L-prolinate was obtained. MS m/z [M+H]⁺ (ESI): 287.10. ¹H NMR (400 MHz, Methanol-d4) δ 7.56-7.50 (m, 1H), 7.35-7.31 (m, 1H), 7.12-6.97 (m, 3H), 4.24-4.10 (m, 2H), 3.31-3.23 (m, 2H), 3.14-2.89 (m, 3H), 2.79-2.67 (m, 1H), 2.56-2.45 (m, 1H), 2.25-2.11 (m, 1H), 1.99-1.83 (m, 3H), 1.27-1.20 (m, 3H).

### Synthesis of 53: (R)-(1-(2-(1H-indol-3-yl)ethyl)azetidin-2-yl)methanol

To a solution of (R)-2-AzetidineMethano (164 mg, 1.89 mmol, 3.00 equiv) and acetic acid (0.1 mL) in methanol (1 mL) were added sodium cyanoborohydride (58.6 mg, 0.95 mmol, 1.50 equiv) at 0° C. To the above mixture was added Aldehyde A (100 mg, 0.63 mmol, 1.00 equiv) in portions over 2 h at 0° C. The reaction was diluted with 10 mL of dichloromethane. The organic layer was washed with 2×10 mL saturated aqueous sodium chloride solution, dried over anhydrous sodium sulfate.

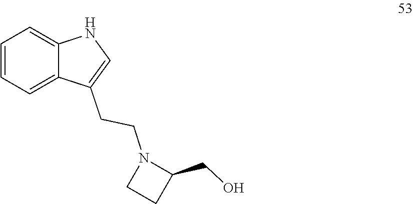 [SMILES: OC[C@H]1CCN1CCc1c[nH]c2ccccc12]

The resulting solution was concentrated under reduced pressure and purified by reverse-phase flash with the following conditions: Column, C18 silica gel; mobile phase, water (with 0.05% NH₄HCO₃) and acetonitrile, 10% to 100% gradient in 20 min; detector, UV 254 nm. 25.2 mg of Compound 53, (R)-(1-(2-(1H-indol-3-yl)ethyl)azetidin-2-yl)methanol was obtained. MS m/z [M+H]⁺ (ESI): 231.25. ¹H NMR (400 MHz, DMSO-d6) δ 10.75 (s, 1H), 7.50-7.45 (m, 1H), 7.32-7.25 (m, 1H), 7.12-7.10 (m, 1H), 7.09-7.01 (m, 1H), 6.99-6.93 (m, 1H), 4.43 (s, 1H), 3.48-3.35 (m, 3H), 3.14-3.06 (m, 1H), 2.90-2.81 (m, 1H), 2.78-2.64 (m, 3H), 2.59-2.52 (m, 1H), 2.00-12.86 (m, 1H), 1.85-1.72 (m, 1H). HRMS m/z [M+H]⁺ (ESI): calc. Mass 231.1497; found 231.1494. ¹³CNMR (75 MHz, Methanol-d₄) δ 136.62, 127.69, 122.87. 121.22, 118.71, 118.54, 113.02, 111.76, 67.95, 68.56, 59.92, 51.37, 23.94, 20.89.

### Synthesis of 54: (S)-(1-(2-(1H-indol-3-yl)ethyl)azetidin-2-yl)methanol

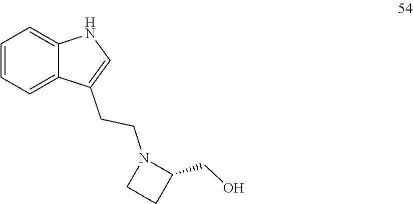 [SMILES: OC[C@@H]1CCN1CCc1c[nH]c2ccccc12]

To a stirred solution of (2S)-azetidin-2-ylmethanol (273 mg, 3.14 mmol, 5.00 equiv) and sodium cyanoborohydride (197 mg, 3.14 mmol, 5.00 equiv) in methanol (1 mL) were added acetic acid (0.10 mL, 1.74 mmol, 2.78 equiv) in portions at 0° C. To the above mixture was added Aldehyde A (100 mg, 0.62 mmol, 1.00 equiv) drop wise over 1 h at 0° C. The resulting mixture was stirred for additional 2 h at room temperature. The reaction was quenched with methanol at room temperature. The resulting mixture was extracted with dichloromethane. The combined organic layers were washed with water. The resulting solution was concentrated under reduced pressure and purified by reverse-phase flash with the following conditions: Column, C18 silica gel; mobile phase, water (with 0.05% ammonium bicarbonate), 10% to 100% gradient in 20 min; detector, UV 254 nm. 26.8 mg of Compound 54, (S)-(1-(2-(1H-indol-3-yl)ethyl)azetidin-2-yl)methanol was obtained. MS m/z [M+H]⁺ (ESI): 231.20. ¹H NMR (400 MHz, Methanol-d₄) δ 7.53-7.49 (m, 1H), 7.31-7.27 (m, 1H), 7.07-7.04 (m, 2H), 7.04-6.99 (m, H), 3.67-3.52 (m, 2H), 3.50-3.48 (m, 2H), 3.10-3.06 (m, 2H), 2.90-2.82 (m, 3H), 2.13-2.05 (m, 2H). HRMS m/z m/z [M+H]+(ESI): calc. Mass 231.1497; found 231.1495. ¹³CNMR (75 MHz, Methanol-d₄) δ 136.75, 127.22, 121.81. 120.91, 118.15, 117.81, 111.82, 110.85, 68.07, 64.21, 59.03, 50.90, 22.84, 19.68.

### Synthesis of 55: isopropyl (2-(1H-indol-3-yl)ethyl)-L-prolinate

Compound 55 was synthesized from product 52, with simple hydrolysis and esterification with isopropyl alcohol. Scheme 6.

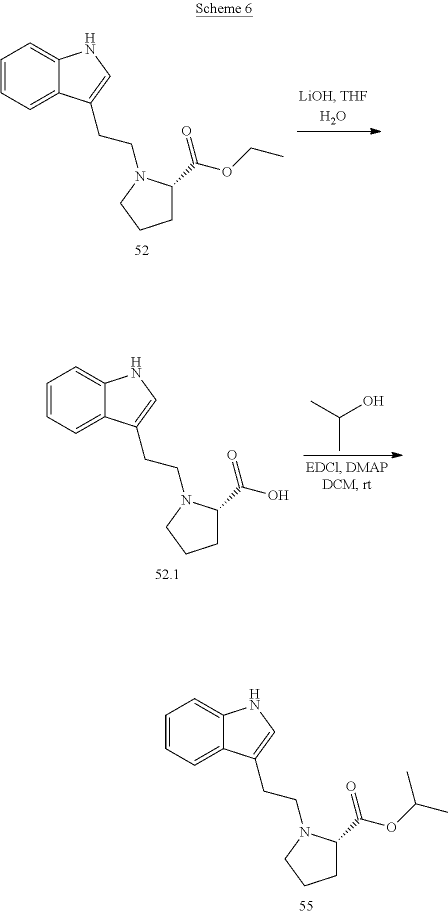

To a stirred solution of 52 (300 mg, 0.78 mmol, 1.00 equiv) and lithium hydroxide (93 mg, 3.90 mmol, 5.00 equiv) in THF (2.10 mL) was added water (0.90 mL, 49.95 mmol, 64.01 equiv) dropwise at room temperature. The resulting mixture was stirred for additional 16h at room temperature. The resulting mixture was concentrated under reduced pressure. The residue was purified by reversed-phase flash chromatography with the following conditions: column, C18 silica gel; mobile phase, acetonitrile in water (with 0.05% ammonium bicarbonate), 10% to 100% gradient in 20 min; detector, UV 254 nm. 100 mg of Compound 52.1 was obtained as a white solid. MS m/z [M+H]⁺ (ESI): 259.14.

To a stirred solution of 52.1 (100 mg, 0.38 mmol, 1.00 equiv) and isopropyl alcohol (116 mg, 1.93 mmol, 5.00 equiv) in N,N-dimethylformamide (1 mL) were added 1-ethyl-3-(3-dimethylaminopropyl)-carbodiimide hydrochloride (111 mg, 0.58 mmol, 1.5 equiv) and 4-dimethylaminopyridine (4 mg, 0.04 mmol, 0.10 equiv) in portions at room temperature. The resulting mixture was stirred for additional 16 h at room temperature. The resulting mixture was extracted with dichloromethane. The combined organic layers were washed with water. The resulting solution was concentrated under reduced pressure and purified by reverse-phase flash with the following conditions: Column, C18 silica gel; mobile phase, water (with 0.05% ammonium bicarbonate), 10% to 100% gradient in 20 min; detector, UV 254 nm. 8.0 mg of 55 was obtained. MS m/z [M+H]⁺ (ESI): 301.25. ¹H NMR (400 MHz, Methanol-d₄) δ 7.57-7.50 (m, 1H), 7.32-7.30 (m, 1H), 7.08-7.03 (m, 2H), 7.00-6.96 (m, 1H), 5.04-5.00 (m, 1H), 3.31-3.25 (m, 2H), 3.23-3.18 (m, 1H), 3.04-2.95 (m, 1H), 2.94-2.90 (m, 1H), 2.73-2.68 (m, 1H), 2.55-2.44 (m, 1H), 2.24-2.11 (m, 1H), 1.91-1.84 (m, 3H), 1.26-1.21 (m, 6H).

### Synthesis of compound 56: (S)-(1-(2-(5-(methoxy-d₃)-1H-indol-3-yl)ethyl)pyrrolidin-2-yl)methanol

Compound 56 was synthesized from commercially available 5-Hydroxyindole in 4 steps reaction Scheme 7.

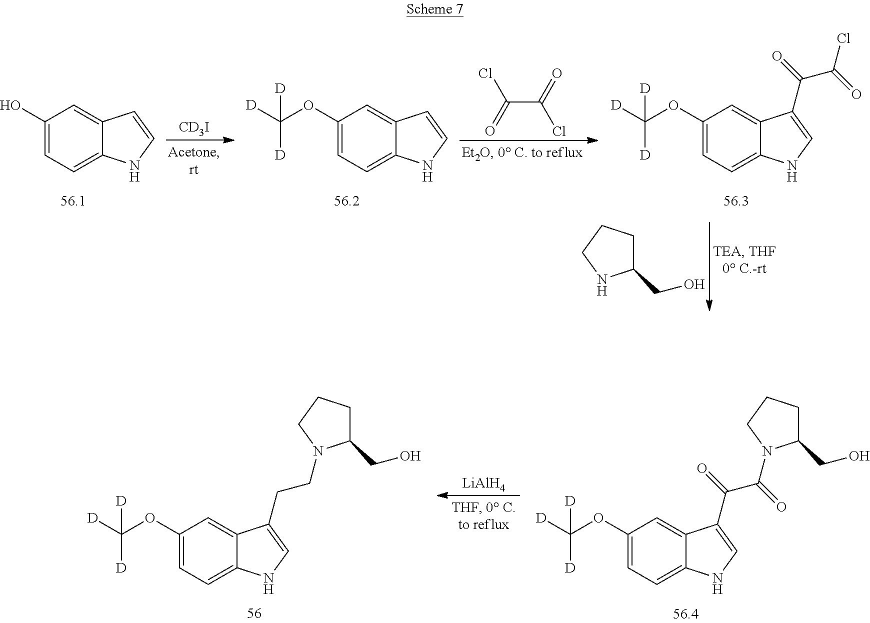

To a solution of 5-Hydroxyindole 56.1 (1 g, 7.5 mmol, 1.0 equiv) in acetone (10 mL), CD₃I (3.3 g, 22.5 mmol, 3.0 equiv) was added at room temperature. The mixture solution was stirred for 1 hour at room temperature and then concentrated under vacuum. The resulting solution was diluted with 20 mL of ethyl acetate and washed with 2×10 mL of saturated NH₄Cl solution and 10 mL of saturated sodium chloride solution respectively. The combined organic layer was washed with 2×20 mL of brine, dried over anhydrous sodium sulfate and concentrated under vacuum. The crude product was purified by Prep-HPLC. 900 mg of compound 56.2 was obtained.

To a solution of compound 56.2 (900 mg, 3 mmol, 1.0 equiv) in THF (10 mL), oxalyl chloride (0.4 mL, 3.1 mmol, 1.03 equiv) was added at room temperature. The mixture solution was stirred for 1 hour at 25° C. and then proline (500 mg, 3.3 mmol, 1.1 equiv) was added in this crude product (compound 56.3). The mixture solution was stirred for 3 hours at 25° C. The resulting solution was diluted with 20 mL of ethyl acetate and washed with 2×10 mL of saturated NaHCO₃ solution and 10 mL of saturated sodium chloride solution respectively. The combined organic layer was washed with 2×20 mL of brine, dried over anhydrous sodium sulfate and concentrated under vacuum. The resulting solution was concentrated under reduced pressure and purified by reverse-phase flash with the following conditions: Column, C18 silica gel; mobile phase, water (with 0.05% HCl), 10% to 100% gradient in 20 min; detector, UV 254 nm. 1 g (50%) of Compound 56.4 was obtained. MS m/z [M+H]⁺ (ESI): 306.35.

A solution/mixture of 56.4 (150 mg) and LiAlH₄ (100 mg, 3 equiv) in THF was stirred for 12 hours at 65° C. The resulting solution was diluted with 20 mL of ethyl acetate and washed with 2×10 mL of saturated NH₄Cl solution. The combined organic layer was washed with 2×20 mL of brine, dried over anhydrous sodium sulfate and concentrated under vacuum. The resulting solution was concentrated under reduced pressure and purified by reverse-phase flash with the following conditions: Column, C18 silica gel; mobile phase, water (with 0.05% HCl), 10% to 100% gradient in 20 min; detector, UV 254 nm. 68.2 mg of Compound 56, S)-(1-(2-(5-(methoxy-d₃)-1H-indol-3-yl)ethyl)pyrrolidin-2-yl)methanol was obtained. MS m/z [M+H]⁺ (ESI): 278.20. ¹H NMR (300 MHz, CD₃OD) δ 7.21-6.74 (m, 4H), 3.66-3.55 (m, 2H), 3.59-3.30 (m, 2H), 3.00-2.76 (m, 5H), 2.02-1.70 (m, 4H). HRMS m/z [M+H]⁺ (ESI): calc. Mass 278.1870; found 278.1419. ¹³CNMR (75 MHz, Methanol-d₄) δ 153.47, 131.98, 127.59. 122.942, 112.32, 111.49, 111.05, 99.88, 65.82, 63.77, 56.16, 54.13, 27.52, 24.00, 22.30.

### Synthesis of compound 57: (S)-(1-(2-(2-bromo-1H-indol-3-yl)ethyl)pyrrolidin-2-yl)methanol

Compound 57 was synthesized from commercially available methyl 2-(1H-indol-3-yl) acetatein 6 simple steps Scheme 8.

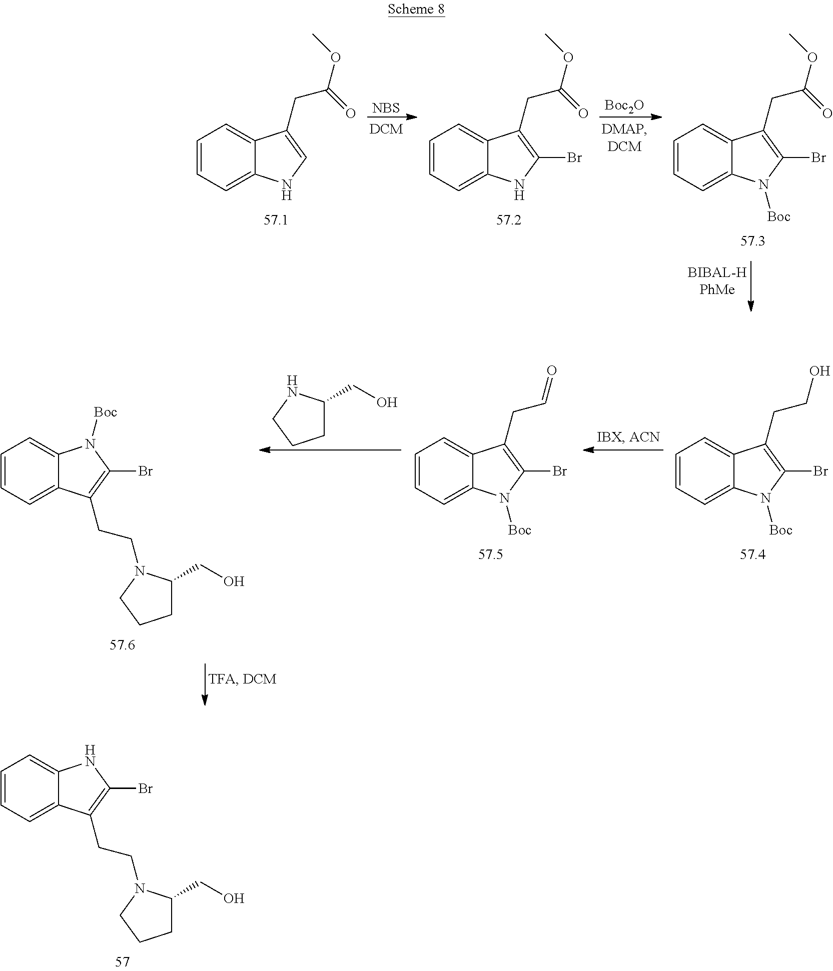

### Synthesis of Intermediate methyl 2-(2-bromo-1H-indol-3-yl) acetate, 57.2

Into a 40 mL vial, to a stirred mixture of methyl 2-(1H-indol-3-yl) acetate, 57.1 (1.0 g, 5.3 mmol, 1 equiv) in DCM was added NBS (940.7 mg, 5.3 mmol, 1.0 equiv) in portions at 0° C. under argon atmosphere. The resulting mixture was stirred for additional 3 h at 0° C. Desired products could be detected by LCMS. The reaction was quenched with ice water at 0° C. The aqueous layer was extracted with EtOAc (4×200 mL). dried over anhydrous Na₂SO₄. After filtration, the filtrate was concentrated under reduced pressure. The residue was purified by silica gel column chromatography, eluted with PE/EA (3:1), to afford 800 mg of methyl 2-(2-bromo-1H-indol-3-yl) acetate, 57.2 which was used directly for the next step without further purification.

### Synthesis of Intermediate tert-butyl 2-bromo-3-(2-methoxy-2-oxoethyl) indole-1-carboxylate, Compounds 57.3

Into a 100 mL round-bottom flask, to a stirred solution methyl 2-(2-bromo-1H-indol-3-yl) acetate 57.2 (800.0 mg, 2.9 mmol, 1.0 equiv) in DCM were added DMAP (36.5 mg, 0.3 mmol, 0.1 equiv) and Boc₂O (781.5 mg, 3.6 mmol, 1.2 equiv) in portions at room temperature under argon atmosphere. The resulting mixture was stirred for additional 3 h at room temperature. Desired product could be detected by LCMS. The resulting mixture was concentrated under vacuum. The residue was purified by silica gel column chromatography, eluted with PE/EA (3:1) to afford 850 mg of tert-butyl 2-bromo-3-(2-methoxy-2-oxoethyl) indole-1-carboxylate, compounds 57.3 which was used directly for the next step without further purification.

### Synthesis of Intermediate tert-butyl 2-bromo-3-(2-hydroxyethyl) indole-1-carboxylate, Compound 57.4

Into a 50 mL 3-necked round-bottom flask, To a stirred mixture of tert-butyl 2-bromo-3-(2-methoxy-2-oxoethyl) indole-1-carboxylate, compounds 57.3 (800.0 mg, 2.2 mmol, 1.0 equiv) in toluene dropwise at room temperature under air atmosphere. To the above mixture was added DIBAL-H (1853.9 mg, 13.0 mmol, 6.0 equiv) dropwise over 2 min at -78° C. under argon atmosphere. The resulting mixture was stirred for additional 2h at −78° C., Desired product could be detected by LCMS. The reaction was quenced with ice water at 0° C.

The precipitated solids were collected by filtration and washed with EtOAc (2×100 mL) and toluene (2×50 mL). dried over anhydrous Na₂SO₄. After filtration, the filtrate was concentrated under reduced pressure. The resulting mixture was concentrated under vacuum. The residue was purified by reversed-phase flash chromatography with the following conditions: column, C18 silica gel; mobile phase, MeCN in Water, 5% to 100% gradient in 30 min; detector, UV 254 nm to afford 540 mg of tert-butyl 2-bromo-3-(2-hydroxyethyl) indole-1-carboxylate, compound 57.4, which was used directly for the next step without further purification.

### Synthesis of Intermediate tert-butyl 2-bromo-3-(2-oxoethyl) indole-1-carboxylate, Compound 57.5

Into a 100 mL round-bottom flask, to a stirred solution of tert-butyl 2-bromo-3-(2-hydroxyethyl) indole-1-carboxylate, compound 57.4 (470.0 mg, 1.4 mmol, 1.0 equiv) in ACN were added IBX (1160.5 mg, 4.1 mmol, 3.0 equiv) in portions at room temperature under argon atmosphere. The resulting mixture was stirred for additional 3h at 80° C. Desired product could be detected by LCMS. The precipitated solids were collected by filtration and washed with ACN (3×50 mL). The resulting mixture was concentrated under vacuum. The residue was purified by silica gel column chromatography, eluted with PE/EA (5:1) to afford 360 mg of tert-butyl 2-bromo-3-(2-oxoethyl) indole-1-carboxylate, compound 57.5, which was used directly for the next step without further purification.

### Synthesis of Intermediate tert-butyl 2-bromo-3-{2-[(2S)-2-(hydroxymethyl) pyrrolidin-1-yl]ethyl} indole-1-carboxylate, Compound 57.6

Into a 40 mL vial, to a stirred solution of [(2S)-pyrrolidin-2-yl] methanol, compound 57.5 (269.2 mg, 2.7 mmol, 2.5 equiv) in MeOH (5.0 mL) were added NaBH₃CN (334.5 mg, 5.3 mmol, 5.0 equiv) and AcOH (0.4 mL, 6.3 mmol, 5.9 equiv) in portions at room temperature under argon atmosphere. To the above mixture was added tert-butyl 2-bromo-3-(2-oxoethyl) indole-1-carboxylate (360.0 mg, 1.1 mmol, 1.0 equiv) in portions over 5 min at 0° C. The resulting mixture was stirred for additional overnight at room temperature. Desired product could be detected by LCMS. The reaction was quenched with ice water at 0° C. The aqueous layer was extracted with EtOAc (4×100 mL). dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 5% to 100% gradient in 30 min; detector, UV 254 nm to afford 270 mg of tert-butyl 2-bromo-3-{2-[(2S)-2-(hydroxymethyl) pyrrolidin-1-yl] ethyl} indole-1-carboxylate, compound 57.6, which was used directly for the next step without further purification.

Into a 40 mL vial, To a stirred solution of tert-butyl 2-bromo-3-{2-[(2S)-2-(hydroxymethyl) pyrrolidin-1-yl] ethyl} indole-1-carboxylate, compound 57.6 (200.0 mg, 0.5 mmol, 1.0 equiv) in DCM (10.0 mL, 157.3 mmol, 332.9 equiv) was added TFA (2.0 mL, 26.9 mmol, 57.0 equiv) dropwise at 0° C. under argon atmosphere. The resulting mixture was stirred for additional 2h at room temperature. Desired product could be detected by LCMS. The reaction was quenched with sat. NaHCO₃ (aq.) at room temperature. The aqueous layer was extracted with CH₂Cl₂ (3×60 mL). dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 5% to 100% gradient in 300 min; detector, UV 254 nm. The crude product (140 mg) was purified by Prep-HPLC with the following conditions (Column: XBridge Shield RP18 OBD Column 30*150 mm, 5m; Mobile Phase A: Water (10 mmol/L NH₄HCO₃), Mobile Phase B: 20 mm NaOH+10% ACN; Flow rate: 60 mL/min mL/min; Gradient: 15% B to 32% B in 8 min; Wave Length: 220 nm/200 nm; RT1(min): 11.1) to afford 52.8 mg of [(2S)-1-[2-(2-bromo-1H-indol-3-yl) ethyl] pyrrolidin-2-yl] methanol, compound 57. MS m/z [M+H]⁺ (ESI): 322.13. ¹H NMR (300 MHz, Methanol-d₄) δ 11.5 (s, 1H), 7.50-7.48 (m, 1H), 7.27-7.21 (m, 1H), 7.12-7.04 (m, 1H), 7.03-7.69 (m, 1H), 4.27 (s, 1H), 3.42-3.25 (m, 2H), 3.20-3.14 (m, 2H), 3.02-2.90 (m, 1H), 2.85-2.70 (m, 2H), 2.50-2.38 (m, 1H), 2.32-2.22 (m, 1H), 1.82-1.75 (m, 1H), 1.73-1.57 (m, 2H), 1.56-1.49 (m, 1H).

### Synthesis of Compound 59: (S)-(1-(2-(5-fluoro-1H-indol-3-yl)ethyl)pyrrolidin-2-yl)methanol

Compound 59 was synthesized in two steps reaction from commercially available 2-(5-fluoro-1H-indol-3-yl) ethanol, Scheme 9.

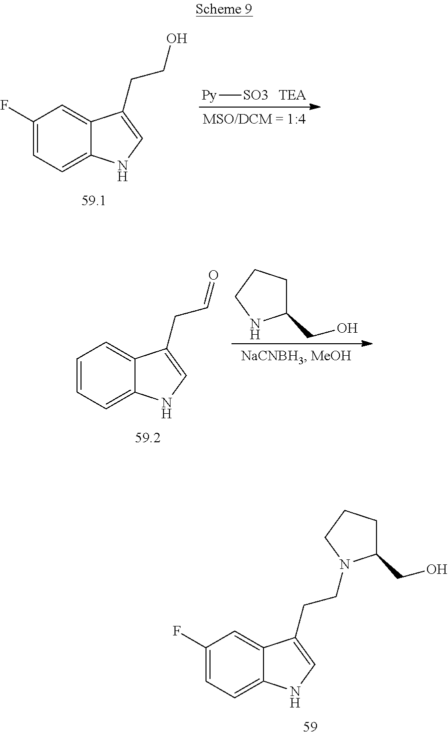

To a stirred solution of 2-(5-fluoro-1H-indol-3-yl) ethanol, 59.1 (400 mg, 2.23 mmol, 1.00 equiv) and pyridine sulfur trioxide (1.776 g, 11.16 mmol, 5.00 equiv) in dichloromethane (16 mL) and dimethyl sulfoxide (4 mL) at 0° C. under nitrogen atmosphere. The resulting mixture was stirred for 2 h at room temperature under nitrogen atmosphere. The organic layer was washed with 2×20 mL saturated aqueous sodium chloride solution, dried over anhydrous sodium sulfate. The residue was purified by silica gel column chromatography, eluted with PE/EA (3:1) to afford 100 mg of 59.2 MS m/z [M+H]+ (ESI): 178.06.

To a stirred solution of prolinol (142 mg, 1.41 mmol, 2.50 equiv) and sodium cyanoborohydride (177 mg, 2.82 mmol, 5.00 equiv) in methanol (1 mL) was added 59.2 (100 mg, 0.56 mmol, 1.00 equiv) in portions at 0° C. under nitrogen atmosphere. The reaction was diluted with 20 mL of ethyl acetate. The organic layer was washed with 2×20 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% ammonium bicarbonate), 10% to 100% gradient in 20 min; detector, UV 254 nm to afford 27.7 mg of compound 59, (S)-(1-(2-(5-fluoro-1H-indol-3-yl)ethyl)pyrrolidin-2-yl)methanol. MS m/z [M+H]⁺ (ESI): 263.20. ¹H NMR (400 MHz, Methanol-d₄) δ 7.28-7.26 (m, 1H), 7.21-7.18 (m, 1H), 7.13 (s, 1H), 6.87-6.82 (m, 1H), 3.65-3.60 (m, 1H), 3.56-3.35 (m, 1H), 3.34-3.32 (m, 1H), 3.31-3.30 (m, 1H), 3.25-2.93 (m, 2H), 2.77-2.70 (m, 2H), 2.53-2.51 (m, 1H), 2.02-1.98 (m, 1H), 1.86-1.84 (m, 2H), 1.83-1.70 (m, 1H). HRMS m/z [M+H]⁺ (ESI): calc. Mass 263.1560; found 263.1569. ¹³CNMR (75 MHz, Methanol-d₄) δ 158.93, 133.26, 127.65. 123.74, 112.86, 112.79, 111.62, 111.49, 109.07, 108.72, 102.57, 102.26, 65.79, 63.76, 56.13, 54.11, 27.52, 23.91, 22.30.

### Synthesis of Compound 61: (S)-(1-(2-(5-(trifluoromethoxy)-1H-indol-3-yl)ethyl)pyrrolidin-2-yl)methanol

Compound 61 was synthesized in three steps reaction from commercially available 5-(trifluoromethoxy)-1H-indole, Scheme 10.

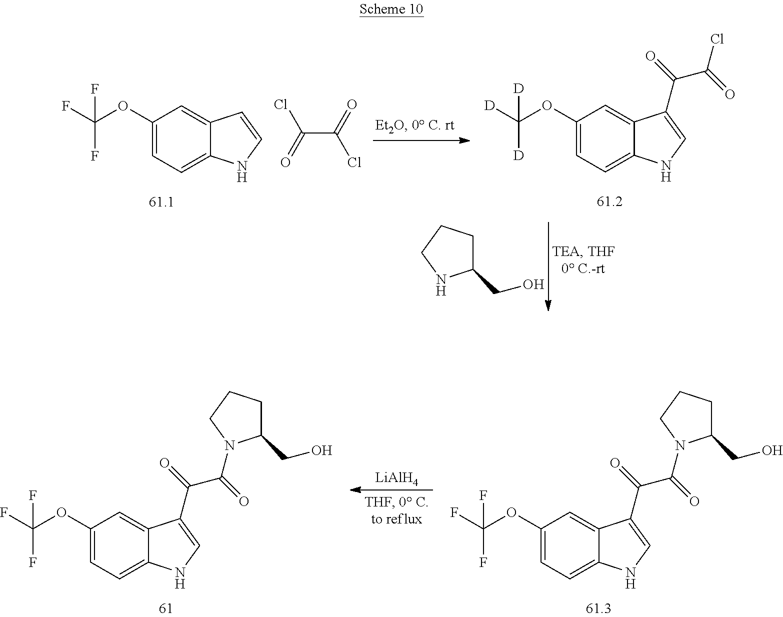

To a solution of 5-(trifluoromethoxy)-1H-indole 61.1 (500 mg, 2.486 mmol, 1 equiv) in diethyl ether (10 mL) was added oxalyl chloride (315.49 mg, 2.486 mmol, 1 equiv) by dropwise at 0° C. The mixture was stirred for one hour. Crude material (61.2) was used at next step directly.

To a solution of prolinol (625.22 mg, 6.180 mmol, 2.5 equiv) and triethylamine (1250.99 mg, 12.360 mmol, 5 equiv) in tetrahydrofuran (10 mL) was added compound 61.2 in diethyl ether by dropwise at 0° C. The mixture was stirred for 16 hours at room temperature. Then, the solvent was removed under reduced pressure. The residue was purified by reversed-phase flash chromatography with the following conditions: column, C18 silica gel; mobile phase, acetonitrile in water (0.05% NH₄HCO₃), 10% to 100% gradient in 20 min; detector, UV 254 nm. This resulted in 400 mg of compound 61.3. MS m/z [M+H]⁺ (ESI): 357.05.

To a solution of compound 61.3 (100 mg, 0.281 mmol, 1 equiv) in tetrahydrofuran (2 mL) was added LiAlH₄ (26.63 mg, 0.703 mmol, 2.5 equiv) at 0° C. The mixture was stirred for 16 hours. It was cooled to 0° C. and quenched with water. The residue was purified by reversed-phase flash chromatography with the following conditions: column, C18 silica gel; mobile phase, acetonitrile in water (0.05% NH₄HCO₃), 10% to 100% gradient in 20 min; detector, UV 210 nm. This resulted in 22 mg of (S)-(1-(2-(5-(trifluoromethoxy)-1H-indol-3-yl)ethyl)pyrrolidin-2-yl)methanol, compound 61. MS m/z [M+H]⁺ (ESI): 329.05. ¹H NMR (400 MHz, Methanol-d₄) δ 8.20-8.13 (m, 1H), 8.03-7.95 (m, 1H), 7.24 (s, 1H), 7.13-7.04 (m, 1H), 3.71 (s, 3H), 3.31-3.23 (m, 2H), 3.12-2.88 (m, 3H), 2.81-2.67 (m, 1H), 2.54-2.43 (m, 1H), 2.25-2.11 (m, 1H), 1.99-1.83 (m, 3H). ¹⁹F NMR (400 MHz, Methanol-d₄) 5-59.62. HRMS m/z [M+H]⁺ (ESI): calc. Mass 329.1477; found 329.1474. ¹³CNMR (75 MHz, Methanol-d₄) δ 142.19, 135.06, 127.44. 124.10, 119.26, 114.57, 113.26, 111.58, 110.30, 65.83, 63.70, 56.11, 54.14, 27.49, 23.78, 22.30.

### Synthesis of 67: (1-(2-(2-bromo-4-methoxy-1H-indol-3-yl)ethyl)pyrrolidin-2-yl)methanol

Compound 67 was prepared from previously synthesized compound 28 in 4 steps reaction, Scheme 11.

To a solution of compound 28 (500 mg, 1.822 mmol, 1 equiv) in dichloromethane (10 mL) was added triethylamine (276.62 mg, 2.733 mmol, 1.5 equiv) and t-butyldimethylchlorosilane (302.14 mg, 2.004 mmol, 1.1 equiv). The mixture was stirred for 16 hours at room temperature. Then, water (10 mL) was added. The aqueous layer was extracted with ethyl acetate (3×10 mL). The combined organic phase was dried over anhydrous Na₂SO₄ and concentrated under reduced pressure. This resulted in 550 mg of compound 67.1. MS m/z [M+H]⁺ (ESI): 389.30.

To a solution of 67.1 (550 mg, 1.415 mmol, 1 equiv) in DCM (10 mL) was added sodium hydride (84.91 mg, 2.123 mmol, 1.5 equiv, 60% in oil) at 0° C. The mixture was stirred for 30 minutes at rt. Then, benzenesulfonyl chloride (274.94 mg, 1.557 mmol, 1.1 equiv) was added at 0° C., and the mixture was stirred for 2 hours at rt. The reaction was quenched with sat. NH₄Cl (aq.) at 0° C. The aqueous layer was extracted with dichloromethane (2×50 mL). The combined organic phase was dried over anhydrous Na₂SO₄ and concentrated under reduced pressure. The residue was purified by reversed-phase flash chromatography with the following conditions: column, C18 silica gel; mobile phase, tetrahydrofuran in water (0.1% Formic acid), 10% to 50% gradient in 10 min; detector, UV 220 nm. This resulted in 220 mg of compound 77.2. MS m/z [M+H]⁺ (ESI): 529.45.

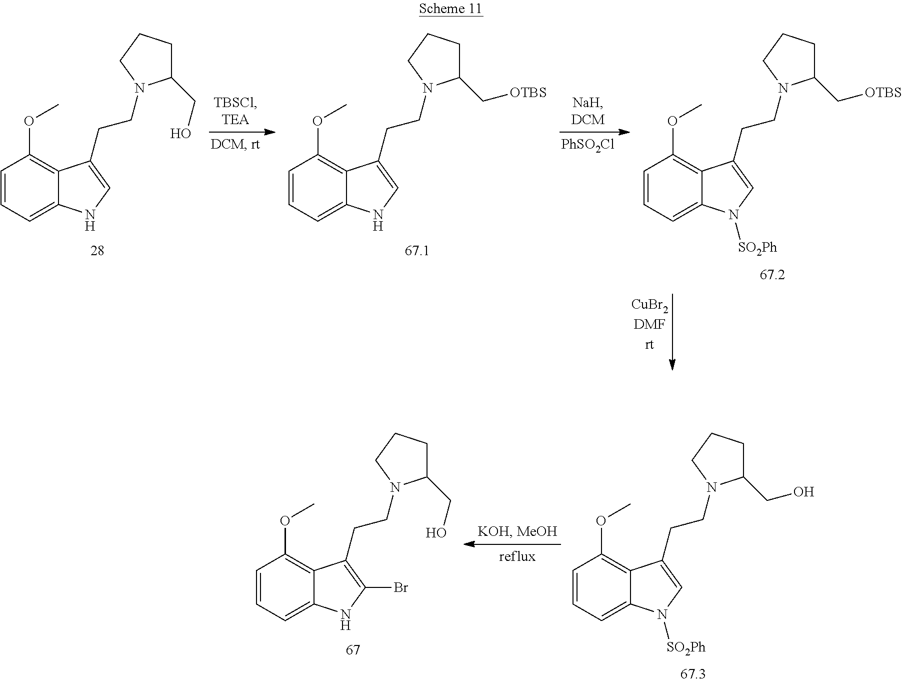

To a solution of 77.2 (210 mg, 0.397 mmol, 1 equiv) in acetonitrile (4.2 mL) was added dibromocopper (798.33 mg, 3.573 mmol, 9 equiv). The mixture was stirred for 3 hours at room temperature. The residue was purified by reversed-phase flash chromatography with the following conditions: column, C18 silica gel; mobile phase, acetonitrile in water (0.1% Formic acid), 5% to 100% gradient in 20 min; detector, UV 210 nm. This resulted in a crude product. The residue was purified by Column: Xselect CSH Prep C18 OBD, 30*150 mm, 5 um; Mobile Phase A: Water (0.1% TFA); Gradient: 26% B to 36% B in 10 min; Wave Length: 254 nm/220 nm nm; RT1(min): 8.62. This resulted in 50 mg of compound 77.3. MS m/z [M+H]⁺ (ESI): 493.10, 495.10.

To a solution of 77.3 (50 mg, 0.101 mmol, 1 equiv) in methanol (2.5 mL) was added potassiumol (28.43 mg, 0.505 mmol, 5 equiv). The mixture was refluxed for 2 hours. Then, it was cooled to room temperature. The residue was purified by Column: XBridge Prep C18 OBD Column 30*150 mm, 5 μm; Mobile Phase A: Water (10 mmol/L NH₄HCO₃), Mobile Phase B: ACN; Flow rate: 60 mL/min mL/min; Gradient: 14% B to 29% B in 11 min; Wave Length: 254 nm/220 nm nm; RT1(min): 10.17. This resulted in 3.3 mg of compound 67, (1-(2-(2-bromo-4-methoxy-1H-indol-3-yl)ethyl)pyrrolidin-2-yl)methanol. MS m/z [M+H]⁺ (ESI): 353.05, 355.05. ¹H NMR (300 MHz, DMSO-d₆) δ 11.55 (s, 1H), 6.98 (t, J=8.1 Hz, 1H), 6.86 (d, J=8.1 Hz, 1H), 6.48 (d, J=7.8 Hz, 1H), 4.30 (s, 1H), 3.84 (s, 3H), 3.48-3.38 (m, 2H), 3.24-3.09 (m, 2H), 3.02-2.73 (m, 3H), 2.44-2.25 (m, 2H), 1.89-1.75 (m, 1H), 1.72-1.50 (m, 3H).

### Synthesis of 69: (1-(2-(2-bromo-5-fluoro-1H-indol-3-yl)ethyl)pyrrolidin-2-yl)methanol

Compound 69 was prepared from commercially available aldehyde in 3 steps reaction, Scheme 12.

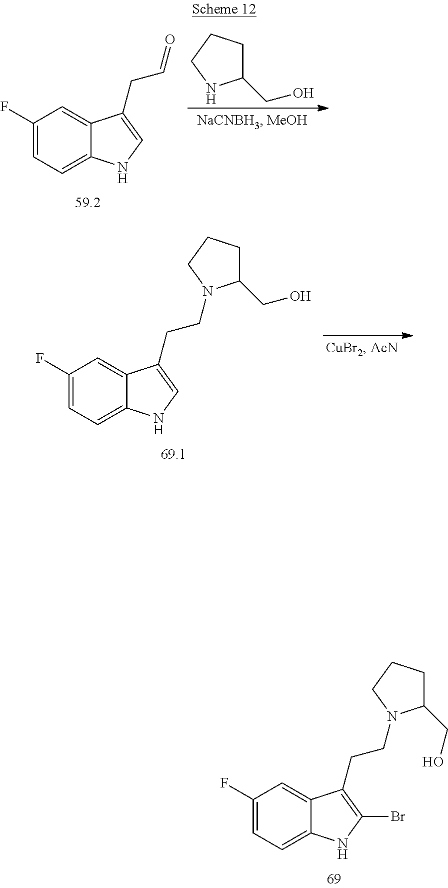

To a stirred solution of prolinol (355 mg, 3.53 mmol, 2.50 equiv) and sodium cyanoborohydride (442 mg, 7.05 mmol, 5.00 equiv) in methanol (3 mL) was added 59.2 (250 mg, 1.41 mmol, 1.00 equiv) in portions at 0° C. under nitrogen atmosphere. The resulting mixture was stirred for 16 h at room temperature under nitrogen atmosphere. The reaction was diluted with 50 mL of ethyl acetate. The organic layer was washed with 2×50 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% HCl), 10% to 100% gradient in 20 min; detector, UV 254 nm to afford 165 mg compound 69.1. MS m/z [M+H]⁺ (ESI): 263.10.

To a stirred solution of compound 69.1. (165 mg, 0.63 mmol, 1.00 equiv) and copper(II) bromide (125 mg, 0.95 mmol, 1.50 equiv) in acetonitrile (3 mL) at 0° C. under nitrogen atmosphere. The resulting mixture was stirred for 3 h at room temperature under nitrogen atmosphere. The reaction was diluted with 100 mL of ethyl acetate. The organic layer was washed with 2×100 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% HCl), 10% to 100% gradient in 20 min; detector, UV 254 nm to afford 31.1 mg of (1-(2-(2-bromo-5-fluoro-1H-indol-3-yl)ethyl)pyrrolidin-2-yl)methanol, compound 69. MS m/z [M+H]⁺ (ESI): 341.00. ¹H NMR (400 MHz, Methanol-d₄) δ 7.31-7.22 (m, 2H), 6.94-6.84 (m, 1H), 3.96-3.85 (m, 1H), 3.83-3.67 (m, 2H), 3.65-3.54 (m, 2H), 3.31-3.09 (m, 4H), 2.32-1.96 (m, 3H), 1.95-1.80 (m, 1H). HRMS m/z [M+H]⁺ (ESI): calc. Mass 341.0665; found 341.0666. ¹³CNMR (75 MHz, Methanol-d₄) δ 159.09, 156.76, 133.17, 127.16, 111.61, 111.51, 110.56, 110.02, 109.75, 108.54, 102.20, 101.95, 120.81, 69.14, 59.67, 54.13, 53.85, 25.79, 22.21, 21.18.

### Synthesis of Compound 70: (1-(2-(2-chloro-5-fluoro-1H-indol-3-yl)ethyl)pyrrolidin-2-yl)methanol

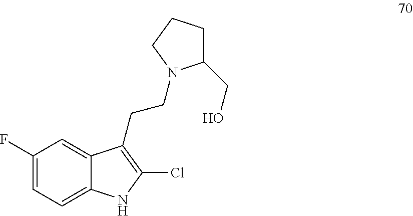 [SMILES: OCC1CCCN1CCc1c(Cl)[nH]c2ccc(F)cc12]

To a stirred solution of 69 (165 mg, 0.29 mmol, 1.00 equiv) in 2M HCl in 1,4-Dioxane (2 mL) at 0° C. under nitrogen atmosphere. The resulting mixture was stirred for 8 h at room temperature under nitrogen atmosphere. The reaction was diluted with 100 mL of ethyl acetate. The organic layer was washed with 2×100 mL saturated aqueous sodium chloride solution, dried over anhydrous sodium sulfate. The resulting solution was concentrated under reduced pressure and purified by reverse-phase flash with the following conditions: Column, C18 silica gel; mobile phase, water (with 0.05% HCl), 10% to 100% gradient in 20 min; detector, UV 254 nm to afford 27.3 mg of (1-(2-(2-chloro-5-fluoro-1H-indol-3-yl)ethyl)pyrrolidin-2-yl)methanol, compound 70 was obtained. MS m/z [M+H]⁺ (ESI): 297.05. ¹H NMR (400 MHz, Methanol-d₄) δ 7.31-7.22 (m, 2H), 6.94-6.84 (m, 1H), 3.96-3.85 (m, 1H), 3.84-3.79 (m, 1H), 3.78-3.56 (m, 3H), 3.40-3.30 (m, 1H), 3.29-2.12 (m, 3H), 2.30-2.12 (m, 2H), 2.11-1.95 (m, 1H), 1.94-1.83 (m, 1H).

### Synthesis of Compound 72: (1-(2-(2-bromo-5-methoxy-1H-indol-3-yl)ethyl)pyrrolidin-2-yl)methanol

Compound 72 was prepared from commercially available 4-methoxy-1H-indole 72.1 in following five steps, Scheme 13.

To a solution of 4-methoxy-1H-indole, 72.1 (10 g, 67.945 mmol, 1 equiv) in diethyl ether (200 mL) was added oxalyl chloride (8.62 g, 67.945 mmol, 1 equiv) by dropwise at 0° C. The mixture was stirred for one hour. This crude material (72.2) was used at next step directly.

To a solution of 2-pyrrolidinemethanol (17.03 g, 168.322 mmol, 2.5 equiv) and TEA (34.07 g, 336.645 mmol, 5 equiv) in tetrahydrofuran (100 mL) was added 72.2 in diethyl ether by dropwise at 0° C. The mixture was stirred for 12 hours at room temperature. Then, the solvent was removed under reduced pressure. The residue was purified by reversed-phase flash chromatography with the following conditions: column, C18 silica gel; mobile phase, acetonitrile in water (0.05% NH₄HCO₃), 10% to 100% gradient in 20 min; detector, UV 254 nm. This resulted in 6 grams of 72.3. MS m/z [M+H]⁺ (ESI): 303.20.

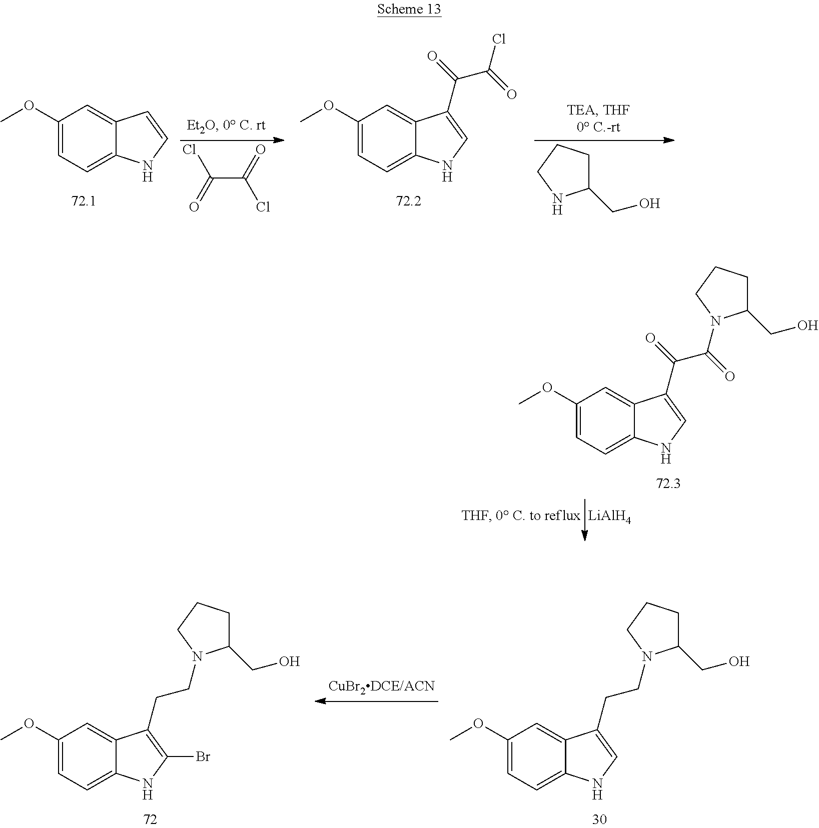

To a solution of 72.3 (6 g, 19.846 mmol, 1 equiv) in tetrahydrofuran (120 mL) was added LiAlH₄ (1.88 g, 49.615 mmol, 2.5 equiv) at 0° C. The mixture was refluxed for 32 hours. It was cooled to 0° C. and quenched with saturated Na₂S₂O₃ aqueous solution. The residue was purified by reversed-phase flash chromatography with the following conditions: column, C18 silica gel; mobile phase, acetonitrile in water (0.1% NH₃·H₂O+10 mmol/L NH₄HCO₃), 10% to 100% gradient in 20 min; detector, UV 210 nm. This resulted in 2.5 grams of 72.4. MS m/z [M+H]⁺ (ESI): 275.25.

To a solution of 30 (300 mg, 1.093 mmol, 1 equiv) in 1,2-dichloroethane (3.00 mL, 34.66 equiv) was added the solution of dibromocopper (0.73 g, 3.279 mmol, 3 equiv) in acetonitrile (3 mL) by dropwise at 0° C. Then, the mixture was stirred for one hour at room temperature. The residue was purified by reversed-phase flash chromatography with the following conditions: column, C18 silica gel; mobile phase, acetonitrile in water (0.1% NH₃·H₂O+10 mmol/L NH₄HCO₃), 5% to 50% gradient in 12 min; detector, UV 254 nm. This resulted in a crude product. The residue was purified by column: XBridge Prep OBD C18 Column, 30*150 mm, 5 μm; Mobile Phase A: Water (10 mmol/L HCl), Mobile Phase B: ACN; Flow rate: 60 mL/min mL/min; Gradient: 35% B to 55% B in 12 min; Wave Length: 220 nm nm; RT1(min): 7.52. This resulted in 7.1 mg of 72. MS m/z [M+H]⁺ (ESI): 353.10, 355.10. ¹H NMR (300 MHz, Methanol-d₄) δ 7.30-7.22 (m, 1H), 7.11 (s, 1H), 6.95-6.85 (m, 1H), 3.85 (s, 3H), 3.69-3.61 (m, 1H), 3.50-3.42 (m, 1H), 3.32-3.11 (m, 4H), 2.71-2.57 (m, 2H), 2.56-2.39 (m, 1H), 2.08-1.85 (m, 1H), 1.86-1.54 (m, 3H).

### Synthesis of Compound 73: (S)-1-((S)-1-(2-(1H-indol-3-yl)ethyl)pyrrolidin-2-yl)ethan-1-ol

Compound 73 was prepared from aldehyde A in one steps.

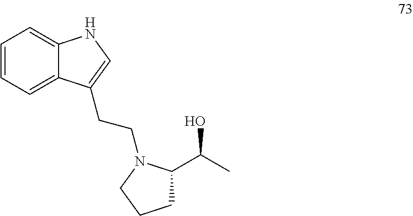 [SMILES: C[C@H](O)[C@@H]1CCCN1CCc1c[nH]c2ccccc12]

Into a 8 mL vial, To a stirred solution of (1S)-1-[(2S)-pyrrolidin-2-yl] ethan-1-ol (144.7 mg, 1.3 mmol, 2 equiv) in MeOH were added NaBH₃CN (197.4 mg, 3.1 mmol, 5.0 equiv) and AcOH (0.1 mL, 1.7 mmol, 2.8 equiv) in portions at room temperature under argon atmosphere. To the above mixture was added indole-3-acetaldehyde A (100.0 mg, 0.6 mmol, 1.0 equiv) in portions over 30 min at 0° C. The resulting mixture was stirred for additional overnight at room temperature. Desired product could be detected by LCMS. The reaction was quenched with ice water at 0° C. The aqueous layer was extracted with EtOAc (4×50 mL). dried over anhydrous Na₂SO₄. After filtration, the filtrate was concentrated under reduced pressure. The crude product (100 mg) was purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 32% B to 49% B in 10 min; Wave Length: 254 nm/220 nm nm; RT1(min): 7.3) to afford 16.7 mg of Compound 73, (1S)-1-[(2S)-1-[2-(1H-indol-3-yl) ethyl]pyrrolidin-2-yl] ethanol. MS m/z [M+H]⁺ (ESI): 258.24. ¹H NMR (300 MHz, Methanol-d4) δ 10.73 (s, 1H), 7.52-7.49 (m, 1H), 7.32-7.29 (m, 1H), 7.15-7.12 (m, 1H), 7.06-7.01 (m, 1H), 7.00-6.94 (m, 1H), 3.62-3.54 (m, 1H), 3.30-3.21 (m, 2H), 2.98-2.84 (m, 2H), 2.80-2.64 (m, 2H), 2.60-2.50 (m, 1H), 1.85-1.70 (m, 2H), 1.71-1.61 (m, 1H), 1.59-1.51 (m, 1H), 1.01-0.96 (m, 3H).

### Synthesis of Compound 74: (S)-1-((S)-1-(2-(1H-indol-3-yl)ethyl)pyrrolidin-2-yl)ethan-1-ol

Compound 74 was prepared from aldehyde A in one steps.

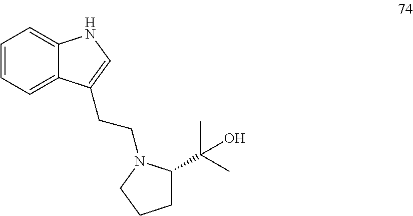 [SMILES: CC(C)(O)[C@@H]1CCCN1CCc1c[nH]c2ccccc12]

A solution of 2-[(2S)-pyrrolidin-2-yl] propan-2-ol (162.3 mg, 1.3 mmol, 2.0 equiv) in MeOH was treated with NaBH₃CN (197.4 mg, 3.1 mmol, 5.0 equiv) and AcOH (0.1 mL) under nitrogen atmosphere followed by the addition of indole-3-acetaldehyde A (100.0 mg, 0.6 mmol, 1.0 equiv) dropwise at 0° C. for 30 min. The resulting mixture was stirred for 2 h at room temperature under nitrogen atmosphere. The reaction was monitored by LCMS. Desired product could be detected by LCMS. The reaction was quenched with Water at room temperature. The resulting mixture was extracted with EtOAc (3×40.0 mL). The combined organic layers were washed with brine (2×50.0 mL), dried over anhydrous Na₂SO₄. After filtration, the filtrate was concentrated under reduced pressure. The crude product (150.0 mg) was purified by Prep-HPLC with the following conditions (Column: Sunfire prep C18 column, 30*150 mm, 5m; Mobile Phase A: Water (0.1% FA), Mobile Phase B: ACN; Flow rate: 60 mL/min mL/min; Gradient: isocratic 2% B t15% B in 10 min; Wave Length: 254 nm/220 nm nm; RT1(min): 12.43) to afford 47.7 mg of Compound 74, 2-[(2S)-1-[2-(1H-indol-3-yl) ethyl] pyrrolidin-2-yl] propan-2-ol. MS m/z [M+H]⁺ (ESI): 273.19. ¹H NMR (300 MHz, Methanol-d₄) 10.85 (s, 1H), 8.23 (s, 1H), 7.58-7.50 (m, 1H), 7.40-7.30 (m, 1H), 7.20-7.15 (m, 1H), 7.12-7.03 (m, 1H), 7.02-6.95 (m, 1H), 3.42-3.25 (m, 2H), 3.09-2.88 (m, 4H), 2.94-2.88 (m, 1H), 1.96-1.71 (m, 4H), 1.23-1.00 (m, 6H). HRMS m/z [M+H]⁺ (ESI): Calc. Mass 273.1967; found 273.1975. ¹³CNMR (75 MHz, Methanol-d₄) δ 164.50, 136.67, 127.42, 123.32. 121.48, 118.77, 118.64, 111.91, 111.33, 74.90, 71.55, 58.63, 55.13, 28.14, 27.00, 25.68, 24.35, 23.09.

### Synthesis of Compound 76, 1-((S)-1-(2-(1H-indol-3-yl)ethyl)pyrrolidin-2-yl)propan-1-ol and the Diastereomeric Pure Compounds 77 (R)-1-((S)-1-(2-(1H-indol-3-yl)ethyl)pyrrolidin-2-yl)propan-1-ol and 78 (S)-1-((S)-1-(2-(1H-indol-3-yl) ethyl)pyrrolidin-2-yl)propan-1-ol

(S)-1-((S)-1-(2-(1H-indol-3-yl)ethyl) pyrrolidin-2-yl)ethan-1-ol. Compound 76 was prepared from aldyhde A in one steps. Compound 76.2 was prepared from commercially available boc protected ester 76.1 and aldehyde A by the following steps. Pure diastereomers were isolated via simple cholun chromatography, Scheme 14.

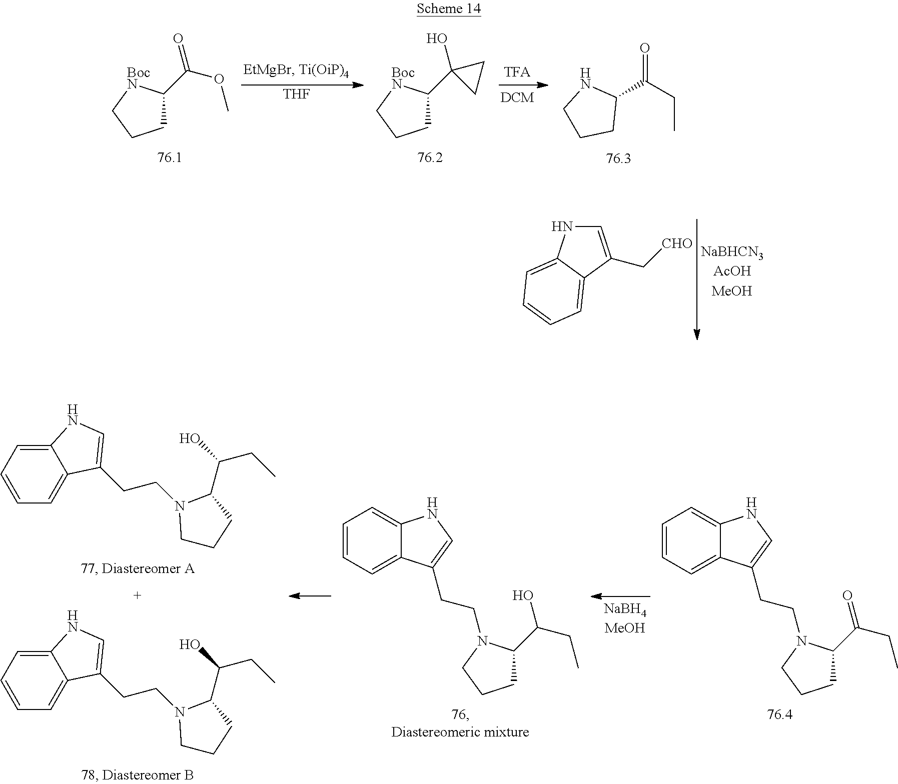

To a stirred mixture of 1-tert-butyl 2-methyl (2S)-pyrrolidine-1,2-dicarboxylate 76.1 (1 g, 4.362 mmol, 1 equiv) in THF were added titanium isopropylate (1 mL) and bromo(methyl)magnesium (3.46 mL) in portions at 0° C. under argon atmosphere. The resulting mixture was stirred for additional 3h at 0° C. Desired products could be detected by LCMS. The reaction was quenched by the addition of sat. NH₄Cl (aq.) (50 mL) at 0° C. The resulting mixture was extracted with EtOAc (3×100 mL). The combined organic layers were washed with brine (1×1 300 mL). The resulting mixture was concentrated under reduced pressure. The residue was purified by silica gel column chromatography, eluted with PE/EA (8:1) to afford 885 mg of compound 76.2, tert-butyl (2S)-2-(1-hydroxycyclopropyl) pyrrolidine-1-carboxylate. This was used directly for the next step reaction.

To a stirred mixture of compound 76.2, tert-butyl (2S)-2-(1-hydroxycyclopropyl) pyrrolidine-1-carboxylate (50 mg, 0.220 mmol, 1 equiv) in TFA (2 mL) in DCM (10 mL) at 0° C. under argon atmosphere. The resulting mixture was stirred for additional 2 h at room temperature. Desired product could be detected by LCMS. The crude product 76.3, was used in the next step directly without further purification.

To a stirred mixture of (S)-1-(pyrrolidin-2-yl) propan-1-one, 76.3 (359.5 mg, 2.8 mmol, 3.0 equiv) in MeOH were added AcOH (0.15 mL, 2.6 mmol, 2.7 equiv) and NaBH₃CN (296.0 mg, 4.7 mmol, 5.0 equiv) in portions at 0° C. under argon atmosphere. To the above mixture was added indole-3-acetaldehyde (150 mg, 0.9 mmol, 1.0 equiv) dropwise over 15 min at 0° C. The resulting mixture was stirred for additional 2h at room temperature. Desired product could be detected by LCMS. The reaction was quenched by the addition of Water/Ice (50 mL) at room temperature. The resulting mixture was extracted with EtOAc (3×100 mL). The combined organic layers were washed with brine (1×300 mL), dried over anhydrous Na₂SO₄. The resulting mixture was concentrated under reduced pressure. The residue was purified by reversed-phase flash chromatography with the following conditions: column, C18 silica gel; mobile phase, MeCN in Water, 10% to 100% gradient in 30 min; detector, UV 254 nm. This resulted in 60 mg of Compound 76.4, 1-(1-(2-(1H-indol-3-yl) ethyl) pyrrolidin-2-yl) propan-1-one.

To a stirred solution of 1-{1-[2-(1H-indol-3-yl) ethyl] pyrrolidin-2-yl} propan-1-one, 76.4, (45 mg, 0.166 mmol, 1 equiv) in MeOH was added NaBH4 (12.59 mg, 0.332 mmol, 2 equiv) in portions at 0° C. under argon atmosphere. The resulting mixture was stirred for additional 2h at room temperature. Desired product could be detected by LCMS. The reaction was quenched by the addition of Water/Ice (30 mL) at room temperature. The resulting mixture was extracted with EtOAc (3×60 mL). The combined organic layers were washed with brine (1×100 mL), dried over anhydrous Na₂SO₄. The resulting mixture was concentrated under reduced pressure. The crude product (45 mg) was purified by Prep-HPLC with the following conditions (Column: XBridge Prep OBD C18 Column 30*150 mm, 5m; Mobile Phase A: Water (10 mmol/L NH₄HCO₃+0.1% NH₃·H₂O), Mobile Phase B: 20 mm NaOH+10% ACN; Flow rate: 60 mL/min mL/min; Gradient: 32% B to 50% B in 10 min; Wave Length: 254 nm/220 nm nm; RT1(min): 6.18/8.16) to afford 7.6 mg of Compound 76. MS m/z [M+H]⁺ (ESI): 273.30

### Compound 77 Diastereomer One: (R)-1-((S)-1-(2-(1H-indol-3-yl)ethyl)pyrrolidin-2-yl)propan-1-ol

MS m/z [M+H]⁺ (ESI): 273.30

¹H NMR (300 MHz, Methanol-d₄) δ 10.73 (s, 1H), 7.50-7.45 (m, 1H), 7.32-7.28 (m, 1H), 7.14-7.12 (m, 1H), 7.05-7.00 (m, 1H), 6.96-6.90 (m, 1H), 4.15-4.10 (m, 1H), 3.16-3.12 (m, 2H), 3.07-3.01 (m, 1H), 2.90-2.72 (m, 2H), 2.63-2.53 (m, 1H), 2.37-2.26 (m, 1H), 1.76-1.64 (m, 2H), 1.62-1.40 (m, 3H), 1.20-1.09 (m, 1H), 0.89-0.85 (m, 3H).

### Compound 78 Diastereomer Second. (S)-1-((S)-1-(2-(1H-indol-3-yl)ethyl)pyrrolidin-2-yl)propan-1-ol

MS m/z [M+H]⁺ (ESI): 273.30

¹H NMR (300 MHz, Methanol-d4) δ 10.76 (s, 1H), 7.52-7.46 (m, 1H), 7.34-7.25 (m, 1H), 7.16-7.12 (m, 1H), 7.06-7.00 (m, 1H), 6.9-6.92 (m, 1H), 3.28-3.20 (m, 2H), 3.05-3.00 (m, 1H), 2.95-2.79 (m, 2H), 2.52-2.47 (m, 1H), 2.41-2.35 (m, 1H), 2.34-2.25 (m, 1H), 1.76-1.54 (m, 4H), 1.42-1.20 (m, 2H), 0.92-0.82 (m, 3H).

### Synthesis of Compound 92: methyl (S)-1-(2-(1H-indol-3-yl)ethyl)-2-methylpyrrolidine-2-carboxylate

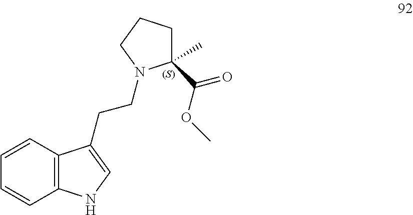

A solution of methyl (2S)-2-methylpyrrolidine-2-carboxylate (134.9 mg, 0.9 mmol, 3.0 equiv) in MeOH was treated with NaBH₃CN (197.4 mg, 3.1 mmol, 5.0 equiv) at room temperature under nitrogen atmosphere followed by the addition of indole-3-acetaldehyde A, (100.0 mg, 0.6 mmol, 1.0 equiv) dropwise at 0° C. for 1 h. The resulting mixture was stirred for 2 h at room temperature under nitrogen atmosphere. The reaction was monitored by TLC. Desired product could be detected by LCMS. The reaction was quenched by the addition of Water (20.0 mL) at room temperature. The resulting mixture was extracted with EtOAc (3×100.0 mL). The combined organic layers were washed with brine (2×100.0 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 5% to 50% gradient in 15 min; detector, UV 254 nm. The crude product (100 mg) was purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 41% B to 58% B in 10 min; Wave Length: 254 nm/220 nm nm; RT1(min): 10.92) to afford 45 mg of methyl (2S)-1-[2-(1H-indol-3-yl) ethyl]-2-methylpyrrolidine-2-carboxylate. MS m/z [M+H]⁺ (ESI): 287.10. ¹H NMR (300 MHz, Methanol-d4) δ 10.75 (s, 1H), 7.46 (m, 1H), 7.32-7.28 (m, 1H), 7.13-7.08 (m, 1H), 7.09-7.01 (m, 1H), 7.01-6.92 (m, 1H), 3.56 (s, 3H), 3.14-3.08 (m, 1H), 2.91-2.66 (m, 4H), 2.15-2.05 (m, 1H), 1.85-1.70 (m, 2H), 1.69-1.59 (m, 1H), 1.21 (s, 3H).

### Synthesis of Compound 93: (S)-(1-(2-(1H-indol-3-yl)ethyl)-2-methylpyrrolidin-2-yl)methanol

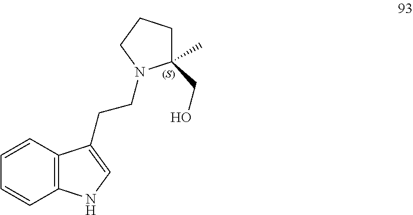

Into a 20 mL vial, to a stirred solution of [(2S)-2-methylpyrrolidin-2-yl] methanol hydrochloride (28.6 mg, 0.2 mmol, 2.0 equiv) in MeOH were added NaBH₃CN (29.6 mg, 0.5 mmol, 5.0 equiv) and AcOH (0.1 mL, 1.7 mmol, 18.5 equiv) in portions at room temperature under argon atmosphere. To the above mixture was added indole-3-acetaldehyde A (100.0 mg, 0.6 mmol, 1.0 equiv) in portions over 30 min at 0° C. The resulting mixture was stirred for additional overnight at room temperature. Desired product could be detected by LCMS. The reaction was quenched with ice water at 0° C. The aqueous layer was extracted with EtOAc (4×50 mL). dried over anhydrous Na₂SO₄. After filtration, the filtrate was concentrated under reduced pressure. The crude product (100 mg) was purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 31% B to 48% B in 10 min; Wave Length: 254 nm/220 nm nm; RT1(min): 9.1) to afford 88.3 mg of Compound 93, [(2S)-1-[2-(1H-indol-3-yl) ethyl]-2-methylpyrrolidin-2-yl] methanol. MS m/z [M+H]⁺ (ESI): 258.22. ¹H NMR (300 MHz, Methanol-d4) δ 10.77 (s, 1H), 7.51-7.46 (m, 1H), 7.33 (d, J=8.1 Hz, 1H), 7.15 (d, J=2.3 Hz, 1H), 7.09-7.05 (m, 1H), 7.01-6.93 (m, 1H), 4.25 (s, 1H), 3.26-3.14 (m, 3H), 2.91-2.74 (m, 3H), 2.71 (d, J=7.9 Hz, 1H), 2.64 (s, 1H), 1.91-1.85 (m, 1H), 1.83-1.61 (m, 2H), 1.50-1.40 (m, 1H), 0.90 (s, 3H).

### Synthesis of Compound 94, methyl (2-(1H-pyrrolo[2,3-b]pyridin-3-yl)ethyl)-L-prolinate and 95 (S)-(1-(2-(1H-pyrrolo[2,3-b]pyridin-3-yl)ethyl)pyrrolidin-2-yl)methanol

Compound 94 and 95 were made from commercially available intermediate 94.1 in following 3 steps, Scheme 15.

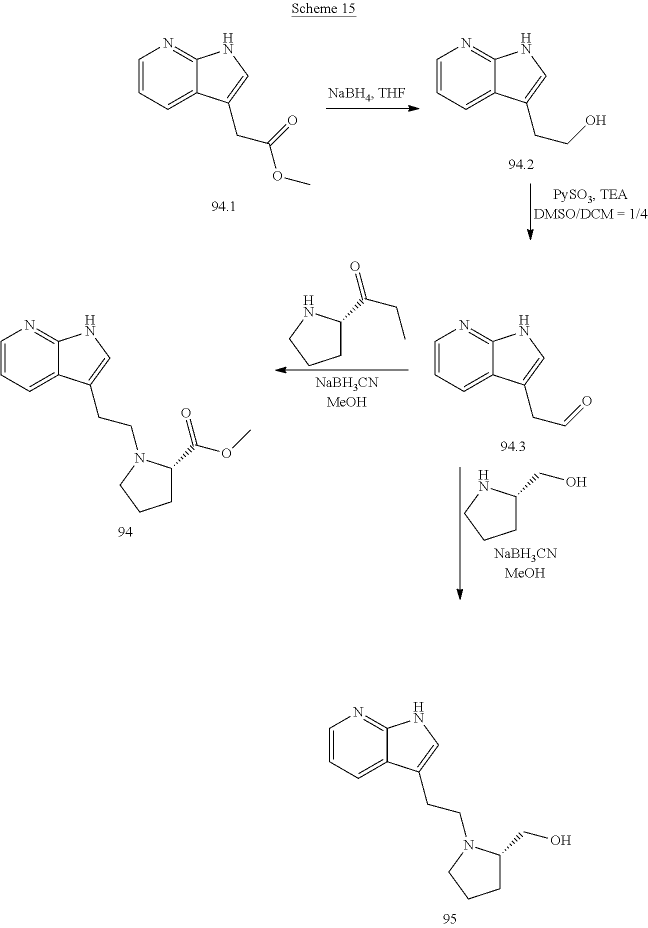

To a solution of methyl 2-{1H-pyrrolo[2,3-b] pyridin-3-yl} acetate, 94.1 (500 mg, 2.629 mmol, 1 equiv) in methanol (5 mL), NaBH₄ (397.79 mg, 10.516 mmol, 4 equiv) was added at 0° C. The mixture was stirred for 16 hours at room temperature. Then, it was quenched with water and the solvent was removed under reduced pressure. The residue was purified by reversed-phase flash chromatography with the following conditions: column, C18 silica gel; mobile phase, acetonitrile in water (0.05% NH₄HCO₃), 10% to 100% gradient in 20 min; detector, UV 254 nm. This resulted in 360 mg of compound 94.2. MS m/z [M+H]⁺ (ESI): 162.95.

To a solution of 94.2 (260 mg, 1.603 mmol, 1 equiv) in dichloromethane (10.4 mL) and dimethyl sulfoxide (2.6 mL), triethylamine (648.87 mg, 6.412 mmol, 4 equiv) and pyridine; sulfonylideneoxidane (510.29 mg, 3.206 mmol, 2 equiv) was added. The mixture was stirred for one hour at room temperature. The residue was purified by silica gel column chromatography, eluted with petroleum ether/ethyl acetate (100%-10%) to afford 120 mg of compound 94.3. MS m/z [M+H]⁺ (ESI): 161.05.

To a mixture of methyl (2S)-pyrrolidine-2-carboxylate hydrochloride (62.04 mg, 0.374 mmol, 2 equiv) and NaBH₃CN (23.54 mg, 0.374 mmol, 2 equiv) in methanol (0.3 mL), Compound 94.3 (30 mg, 0.187 mmol, 1 equiv) in methanol (0.3 mL) was added by dropwise at 0° C. The mixture was stirred for 16 hours at room temperature. The resulting mixture was

[symbol]

filtered. The filtrate was purified by reversed-phase flash chromatography with the following conditions: column, C18 silica gel; mobile phase, acetonitrile in water (0.05% NH₄HCO₃), 10% to 100% gradient in 20 min; detector, UV 210 nm. This resulted in 23.5 mg of compound 94, methyl (2-(1H-pyrrolo[2,3-b]pyridin-3-yl)ethyl)-L-prolinate. MS m/z [M+H]⁺ (ESI): 274.20. H NMR (400 MHz, Methanol-d₄) δ 8.20-8.13 (m, 1H), 8.03-7.95 (m, 1H), 7.24 (s, 1H), 7.13-7.04 (m, 1H), 3.71 (s, 3H), 3.31-3.23 (m, 2H), 3.12-2.88 (m, 3H), 2.81-2.67 (m, 1H), 2.54-2.43 (m, 1H), 2.25-2.11 (m, 1H), 1.99-1.83 (m, 3H).

To a mixture of prolinol (63.15 mg, 0.624 mmol, 2 equiv) and NaBH₃CN (39.23 mg, 0.624 mmol, 2 equiv) in methanol (0.5 mL), Compound 94.3 (50 mg, 0.312 mmol, 1 equiv) in methanol (0.5 mL) was added by dropwise at 0° C. The mixture was stirred for 16 hours at room temperature. The filtrate was purified by reversed-phase flash chromatography with the following conditions: column, C18 silica gel; mobile phase, acetonitrile in water (0.05% NH₄HCO₃), 10% to 100% gradient in 20 min; detector, UV 210 nm. This resulted in 15.1 mg of compound 95, (S)-(1-(2-(1H-pyrrolo[2,3-b]pyridin-3-yl)ethyl)pyrrolidin-2-yl)methanol (15.1 mg, 19%) as a yellow solid. MS m/z [M+H]⁺ (ESI): 246.10. ¹H NMR (400 MHz, Methanol-d₄) δ 8.19-8.12 (m, 1H), 8.09-8.01 (m, 1H), 7.24 (s, 1H), 7.14-7.03 (m, 1H), 3.69-3.57 (m, 1H), 3.54-3.48 (m, 1H), 3.32-3.17 (m, 2H), 3.06-2.90 (m, 2H), 2.71-2.60 (m, 2H), 2.47-2.37 (m, 1H), 2.03-1.93 (m, 1H), 1.88-1.75 (m, 2H), 1.75-1.63 (m, 1H).

TABLE 1
Methods:

[symbol]

[symbol]

indicates data missing or illegible when filed

### GPCR Arrestin Assay Methods

Arrestin Pathway (Performed by DiscoverX Eurofins)

The PathHunter® β-Arrestin assay monitors the activation of a GPCR in a homogenous, non-imaging assay format using a technology developed by DiscoverX called Enzyme Fragment Complementation (EFC) with β-galactosidase (β-Gal) as the functional reporter. The enzyme is split into two inactive complementary portions (EA for Enzyme Acceptor and PK for ProLink) expressed as fusion proteins in the cell. EA is fused to β-Arrestin and PK is fused to the GPCR of interest.

When the GPCR is activated and β-Arrestin is recruited to the receptor, ED and EA complementation occurs, restoring β-Gal activity which is measured using chemiluminescent PathHunter® Detection Reagents.

PathHunter cell lines (DiscoveRx Eurofins) were expanded from freezer stocks according to standard procedures. Cells were seeded in a total volume of 20 μL into white walled, 384-well microplates and incubated at 37° C. for the appropriate time prior to testing.

For agonist determination, cells were incubated with sample to induce response. Intermediate dilution of sample stocks was performed to generate 5× sample in assay buffer. 5 μL of 5× sample was added to cells and incubated at 37° C. or room temperature for 90 to 180 minutes. Vehicle concentration was 1%.

b-Arrestin assay signal was generated through a single addition of 12.5 or 15 μL (50% v/v) of PathHunter Detection reagent cocktail, followed by a one-hour incubation at room temperature. Microplates were read following signal generation with a PerkinElmer Envision™ instrument for chemiluminescent signal detection.

Compound activity was analyzed using CBIS data analysis suite (ChemInnovation, CA). For agonist mode assays, percentage activity was calculated using the following formula:

% Activity=100%×(mean RLU of test sample−mean RLU of vehicle control)/(mean MAX control ligand−mean RLU of vehicle control).

In these studies, the MAX control ligand response was generated using 10 mM serotonin.

### Calcium Mobilization Assay Method (Performed by DiscoveRx Eurofins)

The Calcium No-Wash^{PLUS} assay monitors the activation of a GPCR via Gq secondary messenger signaling in a live cell, non-imaging assay format. Calcium mobilization in PathHunter® cell lines or other cell lines stably expressing Gq-coupled GPCRs is monitored using a calcium-sensitive dye that is loaded into cells. GPCR activation by a compound results in the release of calcium from intracellular stores and an increase in dye fluorescence that is measured in real-time.

Cell lines expressing the GPCR of interest were expanded from freezer stocks according to standard procedures. Cells were seeded in a total volume of 20 μL into black-walled, clear-bottom, Poly-D-lysine coated 384-well microplates and incubated at 37° C. for the appropriate time prior to testing.

Assays were performed in 1× Dye Loading Buffer consisting of 1× Dye, 1× Additive A and 2.5 mM Probenecid in HBSS/20 mM Hepes. Probenicid was prepared fresh. Cells were loaded with dye prior to testing. Media was aspirated from cells and replaced with 20 μL Dye Loading Buffer. Cells were incubated for 30-60 minutes at 37° C.

For agonist determination, cells were incubated with sample to induce response. After dye loading, cells were removed from the incubator and 10 μL HBSS/20 mM Hepes was added. 3× vehicle was included in the buffer when performing agonist dose curves to define the EC80 for subsequent antagonist assays. Cells were incubated for 30 minutes at room temperature in the dark to equilibrate plate temperature.

Intermediate dilution of sample stocks was performed to generate 4× sample in assay buffer. Compound agonist activity was measured on a FLIPR Tetra (MDS). Calcium mobilization was monitored for 2 minutes and 10 μL 4× sample in HBSS/20 mM Hepes was added to the cells 5 seconds into the assay.

Compound activity data was analyzed using CBIS data analysis suite (ChemInnovation, CA). For agonist mode assays, percentage activity is calculated using the following formula:

% Activity=100%×(mean RFU of test sample−mean RFU of vehicle control)/(mean MAX RFU control ligand−mean RFU of vehicle control).

In these studies, the MAX RFU was generated by using 0.1 mM serotonin for the calcium mobilization assay.

IPOne assays were performed using stably-transfected cell lines (CHO-K1) expressing human 5-HT2A or 5-HT2B receptors. Upon activation of these receptors, Gq-mediated myo-Inositol 1 phosphate (IP1) production is detected by a Homogeneous Time-Resolved Fluorescence (HTRF) competitive immunoassay, whereby an IP1 analog coupled to a fluorophore (acceptor) competes with endogenous IP1 for binding to a labeled anti-IP1 antibody (donor). The resulting signal is inversely proportional to the concentration of IP1 in the sample. Cells were incubated with an IP1 inhibitor (to prevent degradation and allow detection) and either reference compound or test compound. α-Me-5-HT was used as the assay reference agonist. Activation of 5-HT2A or 5-HT2B receptors was measured via accumulation of IP1 detected by HTRF. Agonist activity of test compounds was expressed as a percentage of the activity of the reference agonist at its EC100 concentration.

Mouse Head Twitch Response (HTR). Male C57BL/6J mice at 6-8 weeks (Jackson Laboratories) were group housed in a vivarium at UCSD. The room was operated on a reverse light cycle (1900 h on; 0700 h off) with food and water available ad libitum, except during testing. All testing was conducted between 1000 h and 1800 h. Mice were surgically implanted with a small neodymium magnet attached to the cranium and fixed with dental cement. After a minimum 2-week recovery period, the mice were injected intraperitoneally with drug or vehicle and immediately placed in a glass cylinder surrounded by a magnetometer coil and activity was recorded during a 30 min test [4]. Coil voltage was amplified, low pass filtered (2 kHz cutoff), and digitized (20 kHz sampling rate). Head twitches were identified in the recordings using a validated technique based on artificial intelligence [5]. Data were plotted as the average number of HTR recorded during the test for each treatment group and analyzed using a 1-way Analysis of Variance (ANOVA; GraphPad Prism). If there was a significant overall effect of treatment at the p<0.05 level, then a Dunnett's post hoc test was performed to compare each treatment group to the vehicle condition.

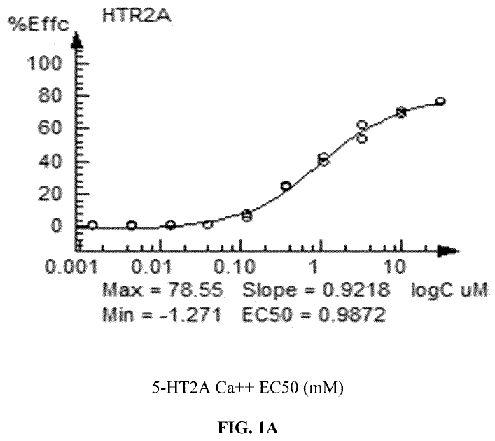

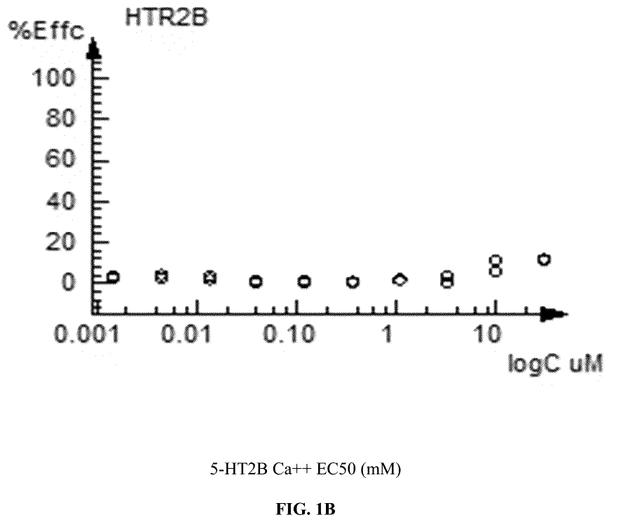

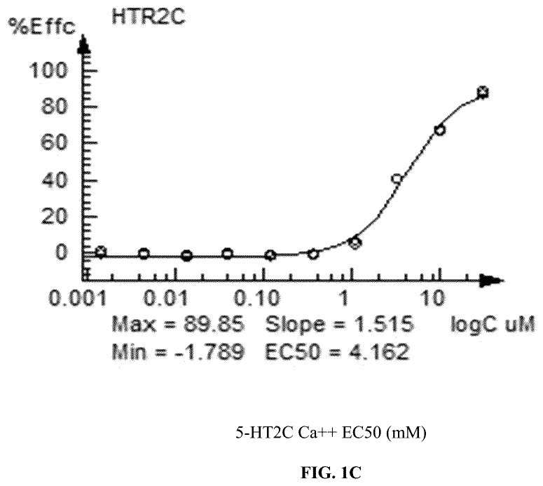

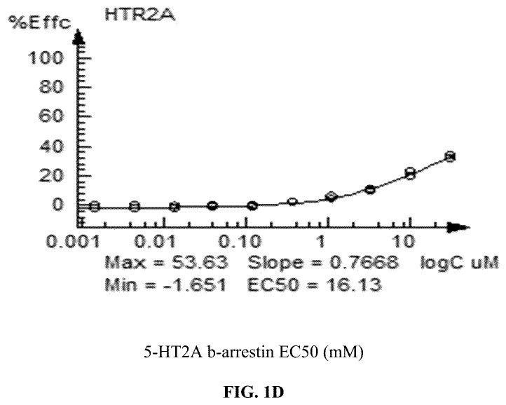

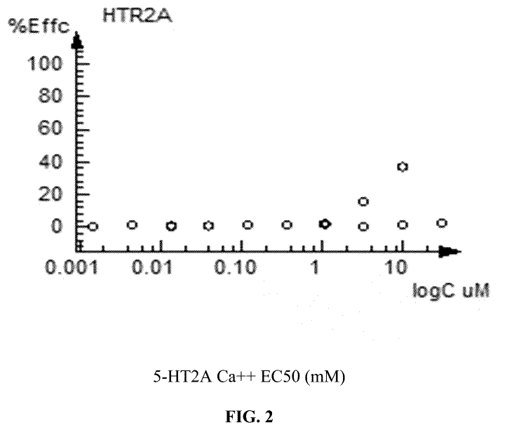

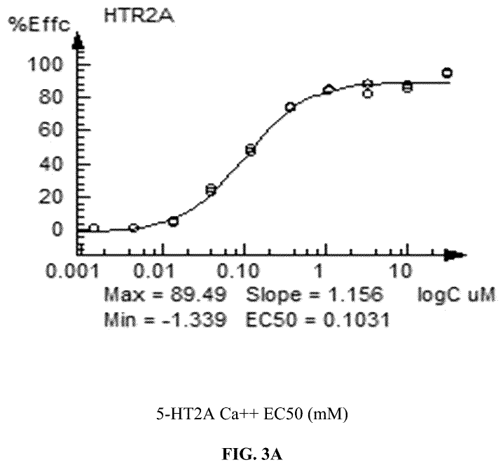

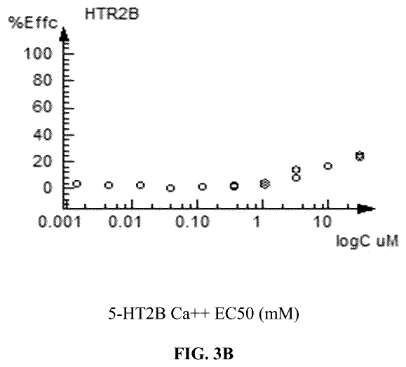

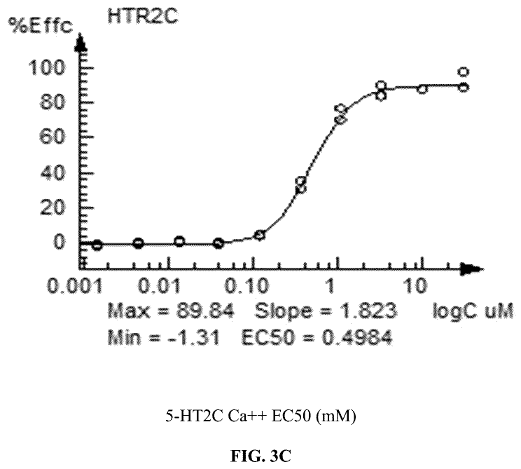

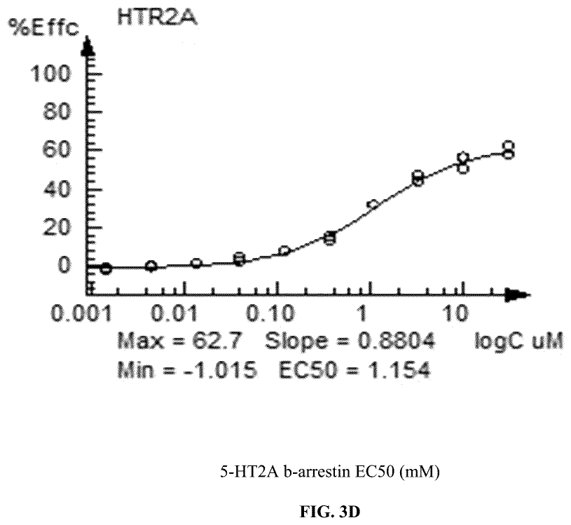

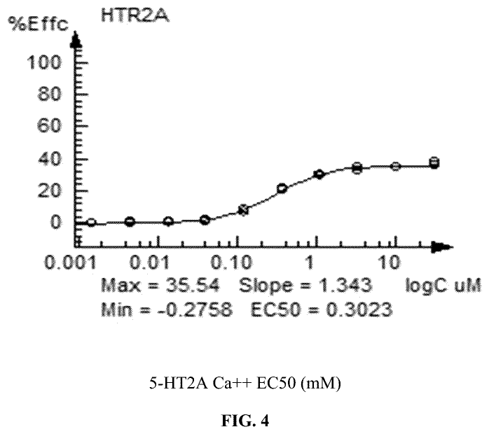

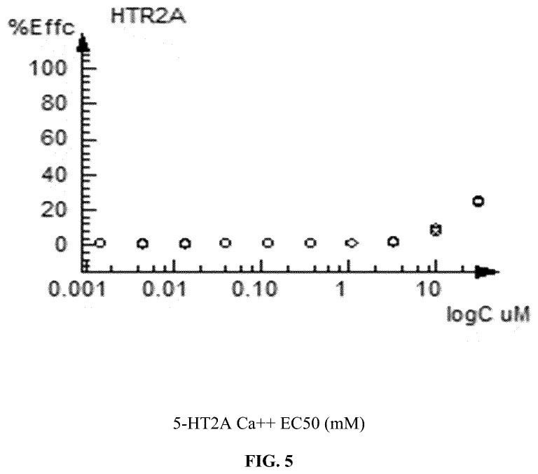

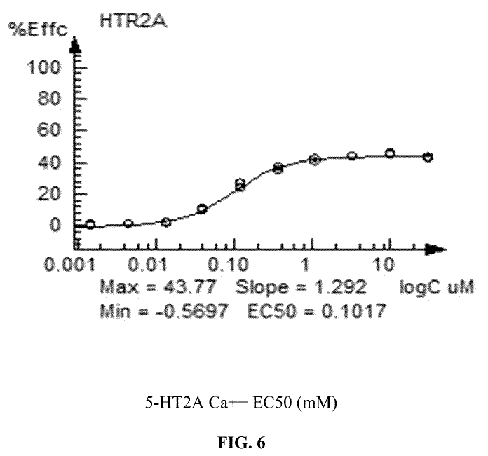

In vitro data for compounds 1-6 are shown in FIGS. 1A-1D (compound 1), compound 1 (FIG. 2), compound 3 (FIGS. 3A-3D), compound 4 (FIG. 4), compound 5 (FIG. 5), and compound 6 (FIG. 6).

1. A compound of Formula (I-C) 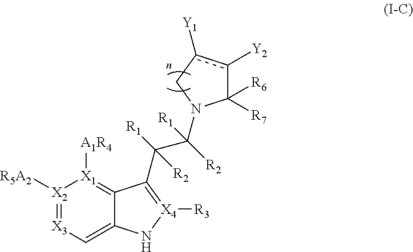
or a pharmaceutically acceptable salt or deuterated form thereof;
wherein,
Y₁ and Y₂ are each independently hydrogen (H), deuterium (D), —CH₃, —OR₁, —CO₂R₈, —NR₁₀R₁₁;
X₁, X₂, X₃, or X₄ are each independently —CH, —C, or —N;
A₁ and A₂ are each independently a bond, H, D, O, N, or S;
R₁, R₂, and R₃ are each independently H, D, alkyl, cycloalkyl, aromatic, halogen, heteroaromatic, or absent;
R₄ and R₅ are each independently H, D, alkyl, cycloalkyl, aromatic, halogen substituted alkyl or cycloalkyl, halogen, heteroaromatic, —C(O)CH₃, or —P(O)(OR₉)₂;
R₆ is H or alkyl;
R₇ is 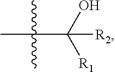
—CO₂R₈, —CH₂C(O)CH₃, —CH₂CO₂R₈, —OP(O)(OR₉)₂, —C(O)—NR₁₀R₁₁;
R₈ is H, alkyl, cycloalkyl, haloalkyl, alkyl-O-alkyl, or aryl;
R₉ is H or alkyl;
R₁₀ and R₁₁ are each independently H or alkyl; and
n is 0, 1, or 2, wherein the compound is not 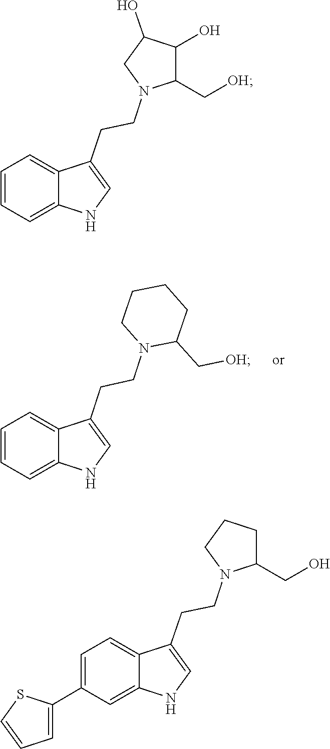 [SMILES: OCC1C(O)C(O)CN1CCc1c[nH]c2ccccc12 ; OCC1CCCCN1CCc1c[nH]c2ccccc12 ; OCC1CCCN1CCc1c[nH]c2cc(-c3cccs3)ccc12]
and wherein when n is 2, R⁷ is not —CO₂R₈ or —CH₂CO₂R₈.

2. The compound of claim 1, wherein Y₁ and Y₂ are each H.

3. The compound of claim 1, wherein X₁ is —C.

4. The compound of claim 1, wherein X₁ is N.

5. The compound of claim 1, wherein X₂ is —C.

6. The compound of claim 1, wherein X₃ is N.

7. The compound of claim 1, wherein X₄ is —C.

8. The compound of claim 1, wherein A₁ is O.

9. The compound of claim 1, wherein A₂ is O.

10. The compound of claim 1, wherein R₁ is H.

11. The compound of claim 1, wherein R₂ is H.

12. The compound of claim 1, wherein R₃ is H.

13. The compound of claim 1, wherein R₃ is halogen.

14. The compound of claim 1, wherein R₄ is H.

15. The compound of claim 1, wherein R₅ is H.

16. The compound of claim 1, wherein A₁ is O and R₄ is H.

17. The compound of claim 1, wherein A₂ is O and R₅ is H.

18. The compound of claim 1, wherein A₁ is O and R₄ is —P(O)(OR₉)₂.

19. The compound of claim 1, wherein A₂ is O and R₅ is —P(O)(OR₉)₂.

20. The compound of claim 1, wherein A₁ is a bond and R₅ is halogen.

21. The compound of claim 1, wherein A₂ is a bond and R₄ is halogen.

22. The compound of claim 1, wherein A₁ is O and R₄ is H.

23. The compound of claim 1, wherein A₂ is O and R₅ is H.

24. The compound of claim 1, wherein A₁ is O and R₄ is alkyl.

25. The compound of claim 1, wherein A₂ is O and R₅ is alkyl.

26. The compound of claim 1, wherein A₁ is O and R₄ is —C(O)CH₃.

27. The compound of claim 1, wherein A₂ is O and R₅ is —C(O)CH₃.

28. The compound of claim 1, wherein R₆ is H.

29. The compound of claim 1, wherein R₆ is alkyl.

30. The compound of claim 29, wherein the alkyl is methyl.

31. The compound of claim 1, wherein R₇ is

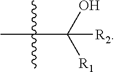

32. The compound of claim 31, wherein R₁ and R₂ are each H.

33. The compound of claim 31, wherein at least one of R₁ or R₂ is alkyl.

34. The compound of claim 31, wherein one of R₁ or R₂ is alkyl and one of R₁ or R₂ is H.

35. The compound of claim 1, wherein R₇ is —CO₂R₈.

36. The compound of claim 35, wherein R₈ is H.

37. The compound of claim 35, wherein R₈ is alkyl.

38. The compound of claim 1, wherein R₇ is —CH₂C(O)CH₃.

39. The compound of claim 1, wherein R₇ is —CH₂CO₂R₈.

40. The compound of claim 39, wherein R₈ is H.

41. The compound of claim 39, wherein R₈ is alkyl.

42. The compound of claim 1, wherein R₇ is —OP(O)(OR₉)₂.

43. The compound of claim 42, wherein R₉ is H.

44. The compound of claim 42, wherein R₉ is alkyl.

45. The compound of claim 1, wherein R₇ is —C(O)—NR₁₀R₁₁.

46. The compound of claim 45, wherein R₁₀ and R₁₁ are each H.

47. The compound of claim 45, wherein at least one of R₁₀ or R₁₁ are alkyl.

48. The compound of claim 45, wherein one of R₁₀ or R₁₁ is alkyl and one of R₁₀ or R₁₁ is H.

49. The compound of claim 1, wherein n is 0.

50. The compound of claim 1, wherein n is 1.

51. The compound of claim 1, wherein n is 2.

52. The compound of claim 1, wherein the compound is:

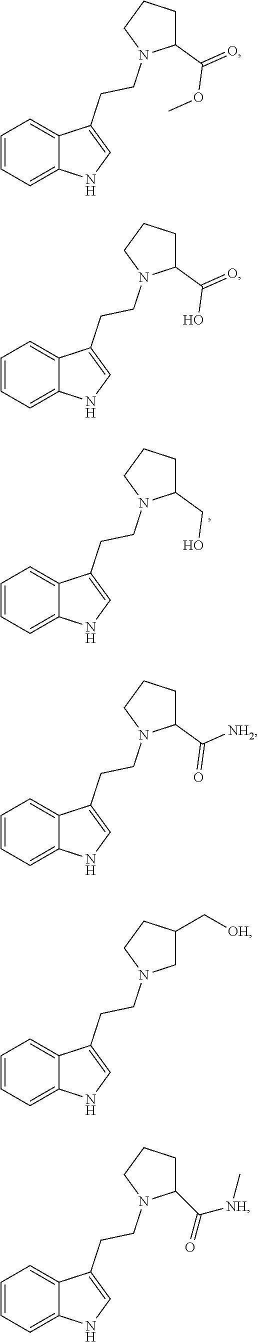 [SMILES: COC(=O)C1CCCN1CCc1c[nH]c2ccccc12 ; O=C(O)C1CCCN1CCc1c[nH]c2ccccc12 ; OCC1CCCN1CCc1c[nH]c2ccccc12 ; NC(=O)C1CCCN1CCc1c[nH]c2ccccc12 ; OCC1CCN(CCc2c[nH]c3ccccc23)C1 ; CNC(=O)C1CCCN1CCc1c[nH]c2ccccc12]

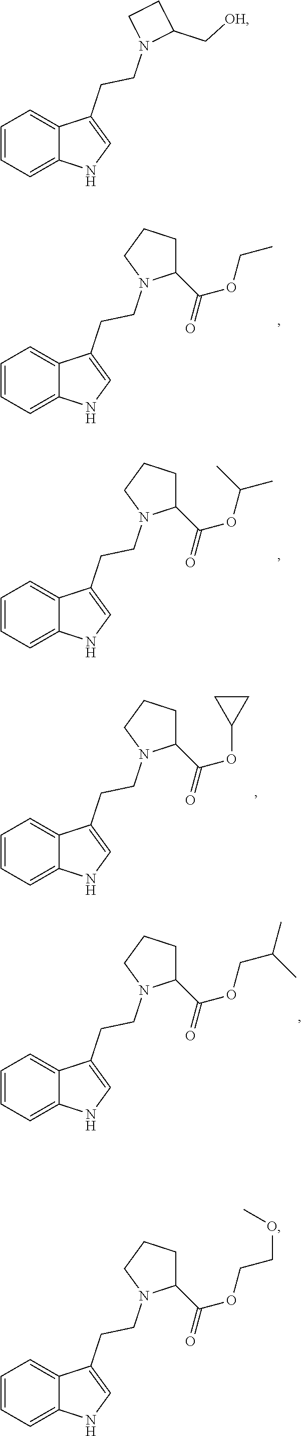 [SMILES: OCC1CCN1CCc1c[nH]c2ccccc12 ; CCOC(=O)C1CCCN1CCc1c[nH]c2ccccc12 ; CC(C)OC(=O)C1CCCN1CCc1c[nH]c2ccccc12 ; O=C(OC1CC1)C1CCCN1CCc1c[nH]c2ccccc12 ; CC(C)COC(=O)C1CCCN1CCc1c[nH]c2ccccc12 ; COCCOC(=O)C1CCCN1CCc1c[nH]c2ccccc12]

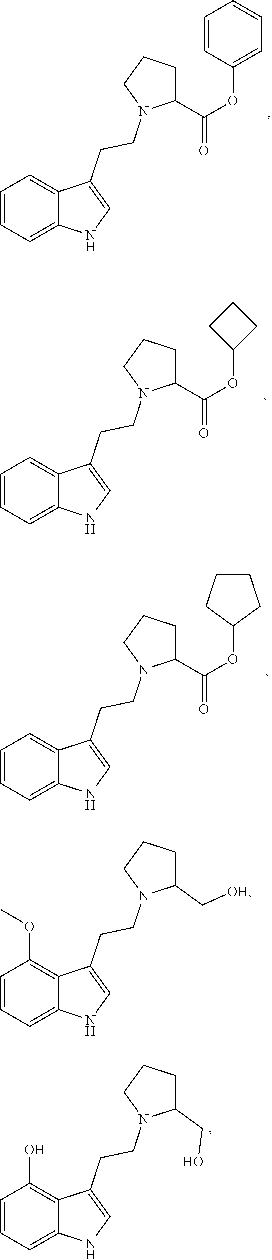 [SMILES: O=C(Oc1ccccc1)C1CCCN1CCc1c[nH]c2ccccc12 ; O=C(OC1CCC1)C1CCCN1CCc1c[nH]c2ccccc12 ; O=C(OC1CCCC1)C1CCCN1CCc1c[nH]c2ccccc12 ; COc1cccc2[nH]cc(CCN3CCCC3CO)c12 ; OCC1CCCN1CCc1c[nH]c2cccc(O)c12]

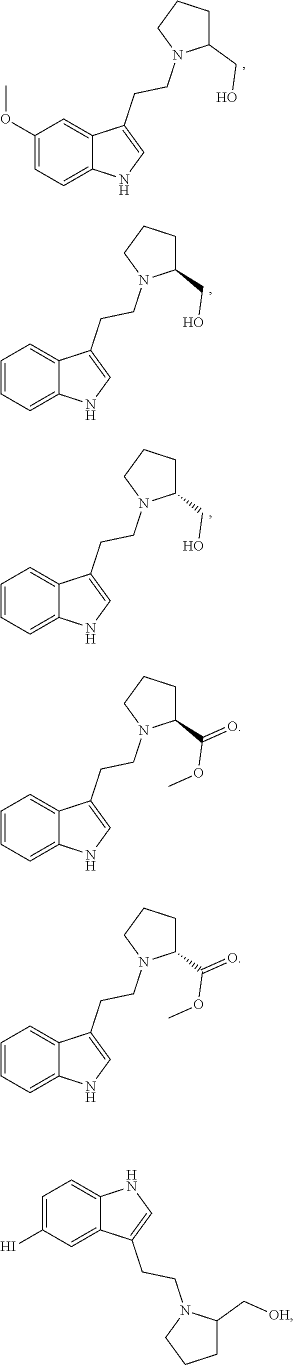 [SMILES: COc1ccc2[nH]cc(CCN3CCCC3CO)c2c1 ; OC[C@@H]1CCCN1CCc1c[nH]c2ccccc12 ; OC[C@H]1CCCN1CCc1c[nH]c2ccccc12 ; COC(=O)[C@@H]1CCCN1CCc1c[nH]c2ccccc12 ; COC(=O)[C@H]1CCCN1CCc1c[nH]c2ccccc12 ; OCC1CCCN1CCc1c[nH]c2ccc(I)cc12]

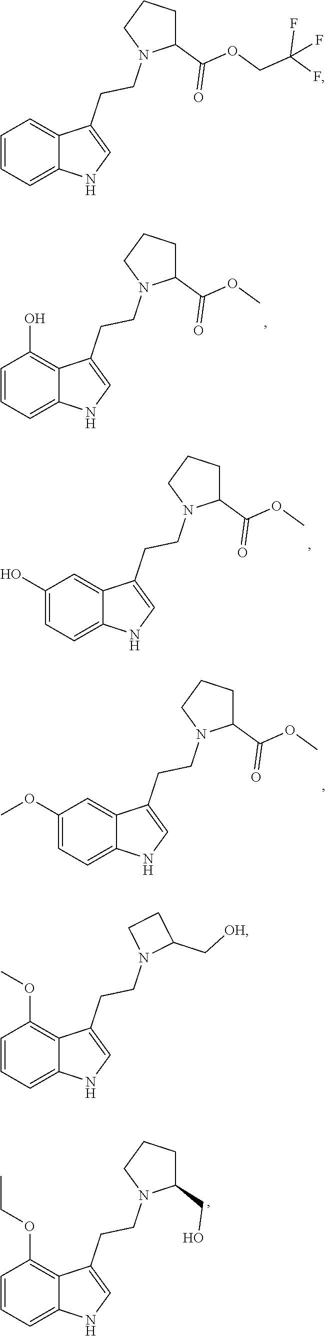

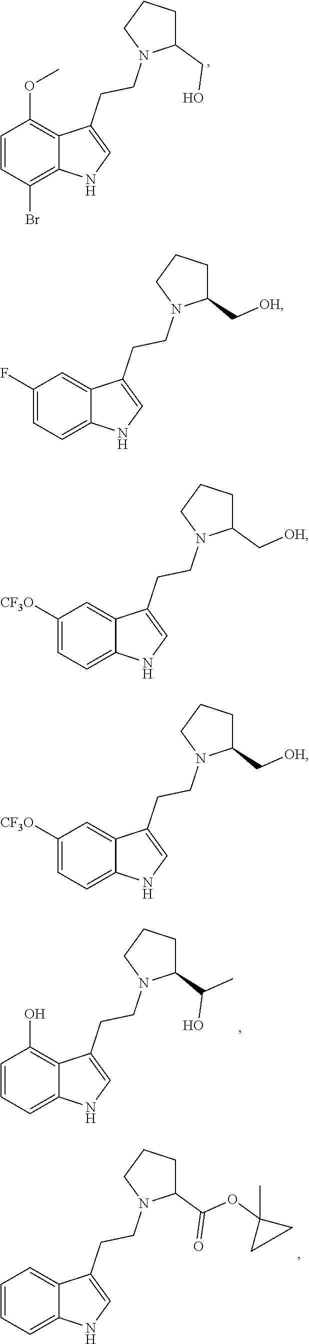

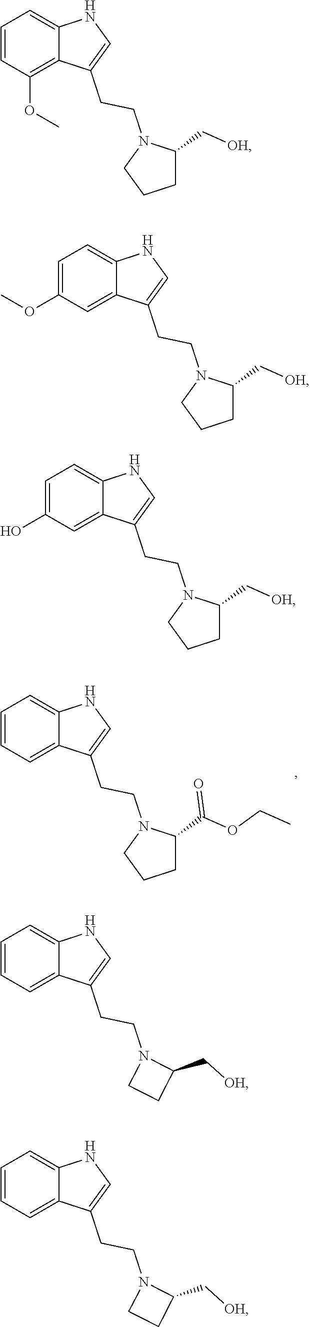 [SMILES: COc1cccc2[nH]cc(CCN3CCC[C@H]3CO)c12 ; COc1ccc2[nH]cc(CCN3CCC[C@H]3CO)c2c1 ; OC[C@@H]1CCCN1CCc1c[nH]c2ccc(O)cc12 ; CCOC(=O)[C@@H]1CCCN1CCc1c[nH]c2ccccc12 ; OC[C@H]1CCN1CCc1c[nH]c2ccccc12 ; OC[C@@H]1CCN1CCc1c[nH]c2ccccc12]

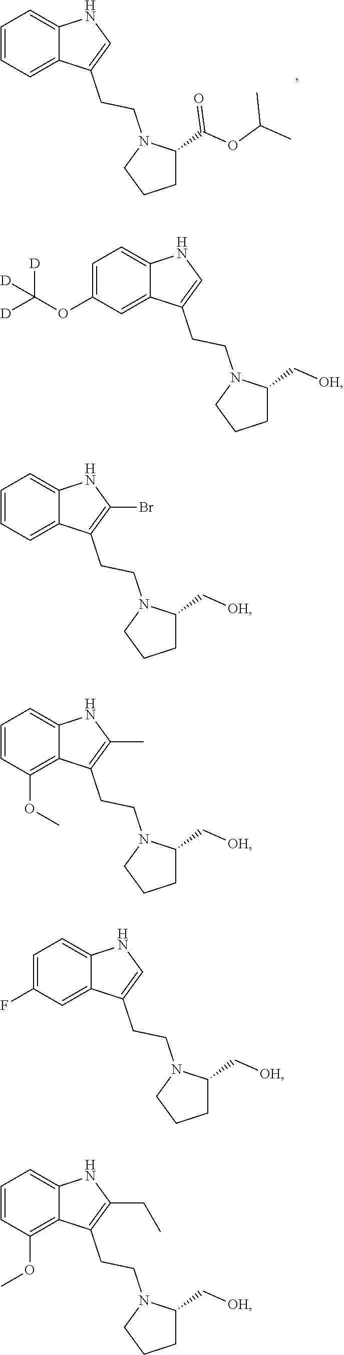 [SMILES: CC(C)OC(=O)[C@@H]1CCCN1CCc1c[nH]c2ccccc12 ; [2H]C([2H])([2H])Oc1ccc2[nH]cc(CCN3CCC[C@H]3CO)c2c1 ; OC[C@@H]1CCCN1CCc1c(Br)[nH]c2ccccc12 ; COc1cccc2[nH]c(C)c(CCN3CCC[C@H]3CO)c12 ; OC[C@@H]1CCCN1CCc1c[nH]c2ccc(F)cc12 ; CCc1[nH]c2cccc(OC)c2c1CCN1CCC[C@H]1CO]

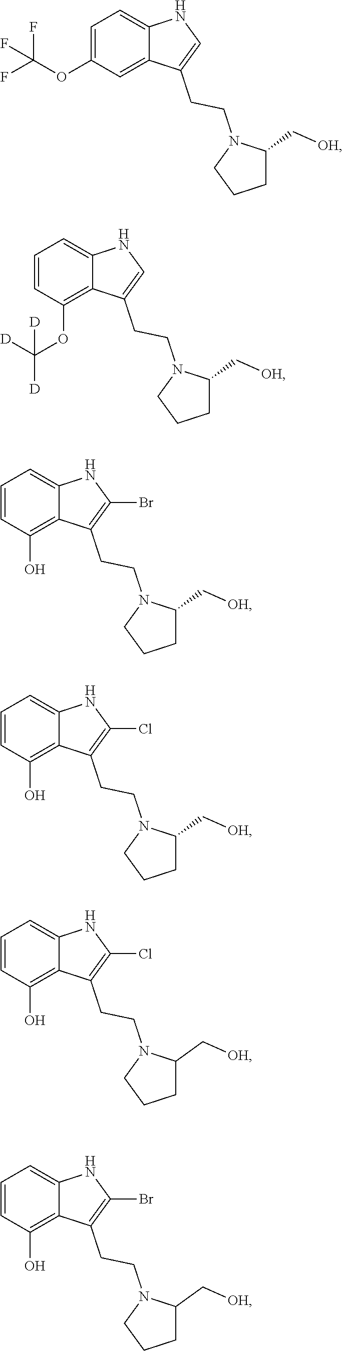 [SMILES: OC[C@@H]1CCCN1CCc1c[nH]c2ccc(OC(F)(F)F)cc12 ; [2H]C([2H])([2H])Oc1cccc2[nH]cc(CCN3CCC[C@H]3CO)c12 ; OC[C@@H]1CCCN1CCc1c(Br)[nH]c2cccc(O)c12 ; OC[C@@H]1CCCN1CCc1c(Cl)[nH]c2cccc(O)c12 ; OCC1CCCN1CCc1c(Cl)[nH]c2cccc(O)c12 ; OCC1CCCN1CCc1c(Br)[nH]c2cccc(O)c12]

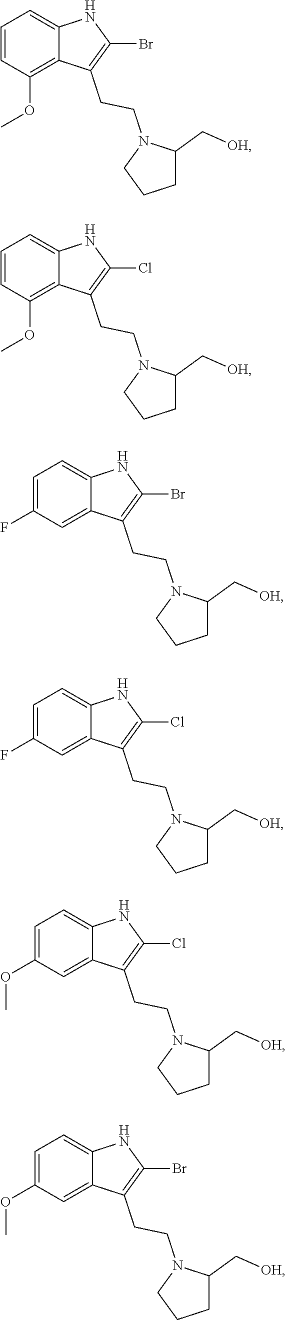 [SMILES: COc1cccc2[nH]c(Br)c(CCN3CCCC3CO)c12 ; COc1cccc2[nH]c(Cl)c(CCN3CCCC3CO)c12 ; OCC1CCCN1CCc1c(Br)[nH]c2ccc(F)cc12 ; OCC1CCCN1CCc1c(Cl)[nH]c2ccc(F)cc12 ; COc1ccc2[nH]c(Cl)c(CCN3CCCC3CO)c2c1 ; COc1ccc2[nH]c(Br)c(CCN3CCCC3CO)c2c1]

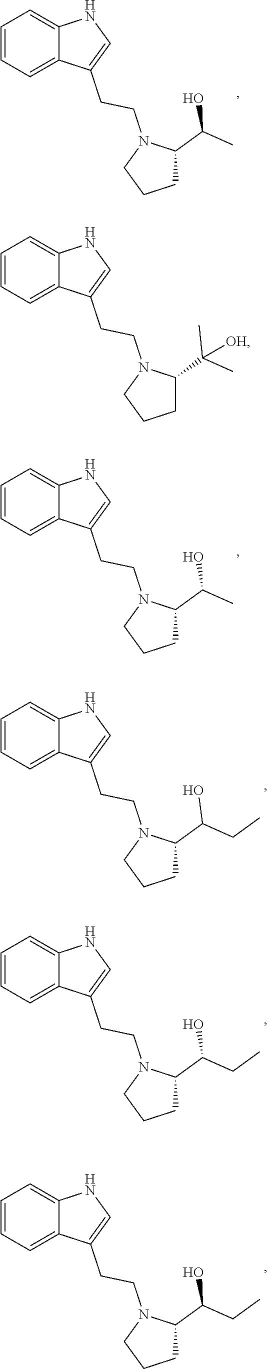 [SMILES: C[C@H](O)[C@@H]1CCCN1CCc1c[nH]c2ccccc12 ; CC(C)(O)[C@@H]1CCCN1CCc1c[nH]c2ccccc12 ; C[C@@H](O)[C@@H]1CCCN1CCc1c[nH]c2ccccc12 ; CCC(O)[C@@H]1CCCN1CCc1c[nH]c2ccccc12 ; CC[C@@H](O)[C@@H]1CCCN1CCc1c[nH]c2ccccc12 ; CC[C@H](O)[C@@H]1CCCN1CCc1c[nH]c2ccccc12]

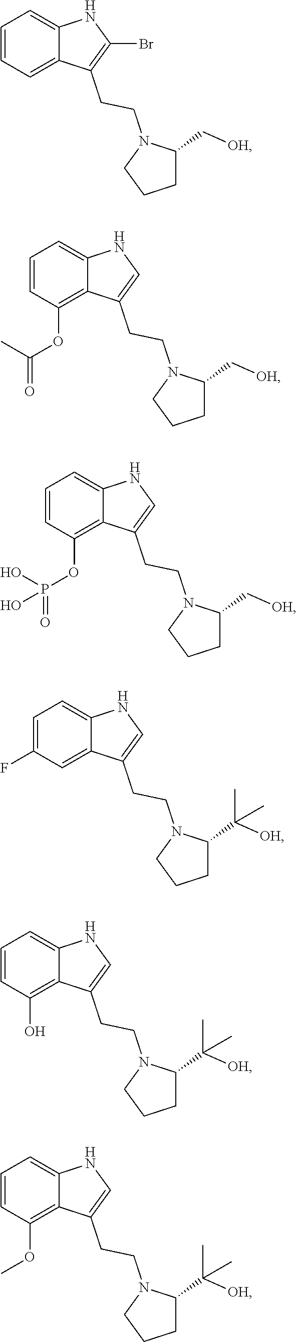 [SMILES: OC[C@@H]1CCCN1CCc1c(Br)[nH]c2ccccc12 ; CC(=O)Oc1cccc2[nH]cc(CCN3CCC[C@H]3CO)c12 ; O=P(O)(O)Oc1cccc2[nH]cc(CCN3CCC[C@H]3CO)c12 ; CC(C)(O)[C@@H]1CCCN1CCc1c[nH]c2ccc(F)cc12 ; CC(C)(O)[C@@H]1CCCN1CCc1c[nH]c2cccc(O)c12 ; COc1cccc2[nH]cc(CCN3CCC[C@H]3C(C)(C)O)c12]

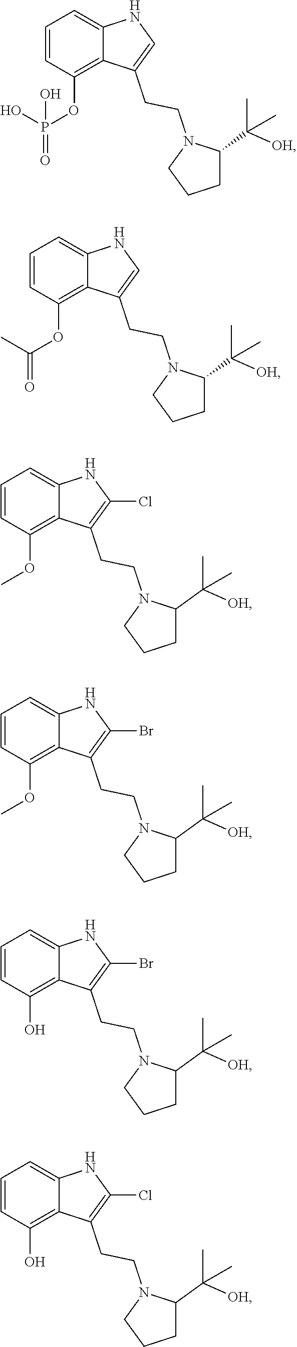 [SMILES: CC(C)(O)[C@@H]1CCCN1CCc1c[nH]c2cccc(OP(=O)(O)O)c12 ; CC(=O)Oc1cccc2[nH]cc(CCN3CCC[C@H]3C(C)(C)O)c12 ; COc1cccc2[nH]c(Cl)c(CCN3CCCC3C(C)(C)O)c12 ; COc1cccc2[nH]c(Br)c(CCN3CCCC3C(C)(C)O)c12 ; CC(C)(O)C1CCCN1CCc1c(Br)[nH]c2cccc(O)c12 ; CC(C)(O)C1CCCN1CCc1c(Cl)[nH]c2cccc(O)c12]

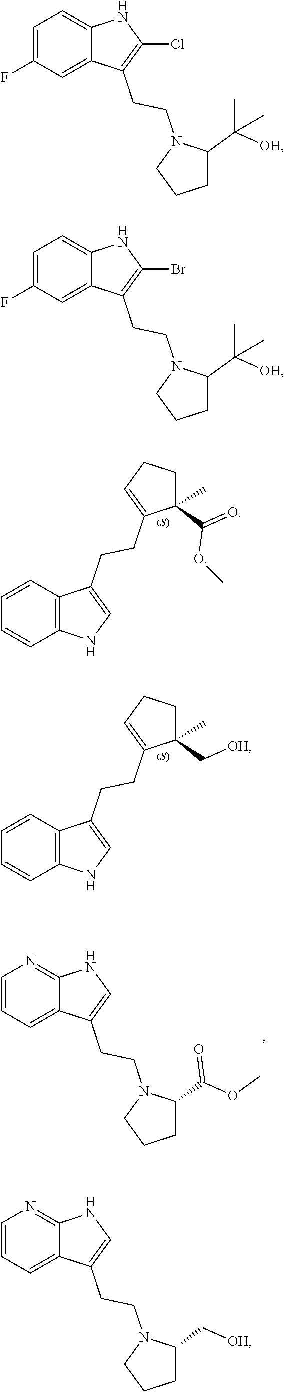

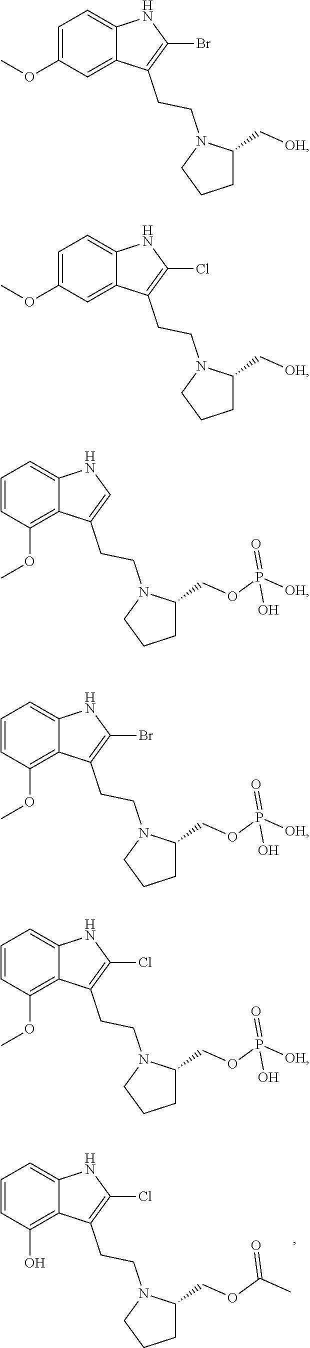 [SMILES: COc1ccc2[nH]c(Br)c(CCN3CCC[C@H]3CO)c2c1 ; COc1ccc2[nH]c(Cl)c(CCN3CCC[C@H]3CO)c2c1 ; COc1cccc2[nH]cc(CCN3CCC[C@H]3COP(=O)(O)O)c12 ; COc1cccc2[nH]c(Br)c(CCN3CCC[C@H]3COP(=O)(O)O)c12 ; COc1cccc2[nH]c(Cl)c(CCN3CCC[C@H]3COP(=O)(O)O)c12 ; CC(=O)OC[C@@H]1CCCN1CCc1c(Cl)[nH]c2cccc(O)c12]

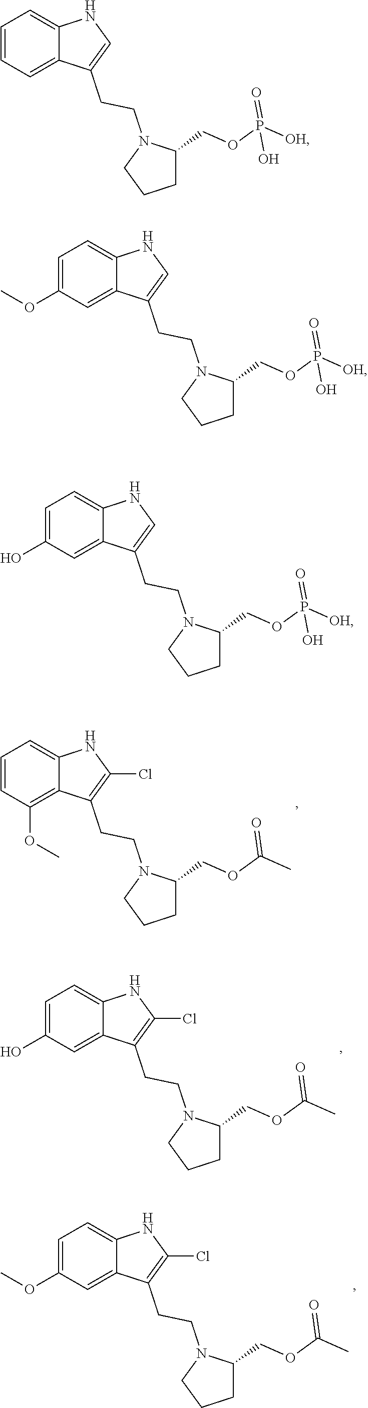

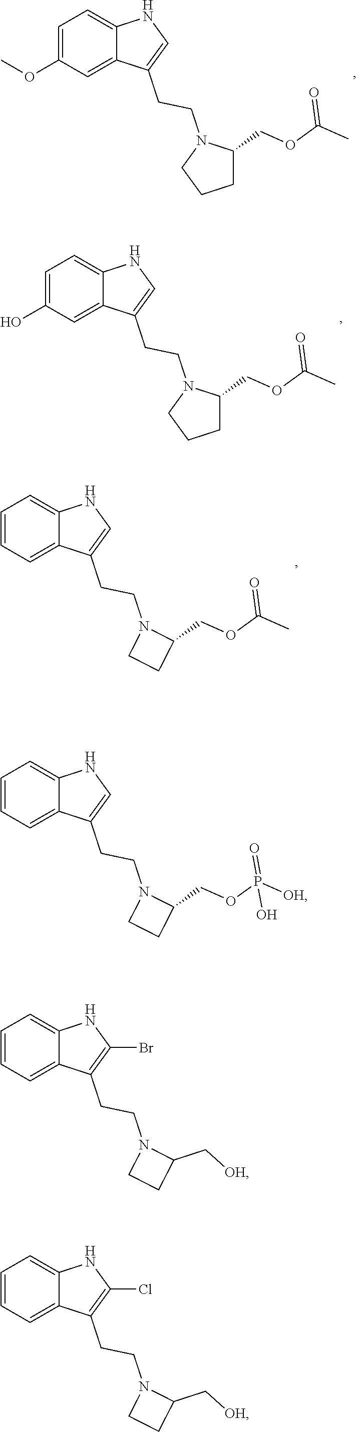 [SMILES: COc1ccc2[nH]cc(CCN3CCC[C@H]3COC(C)=O)c2c1 ; CC(=O)OC[C@@H]1CCCN1CCc1c[nH]c2ccc(O)cc12 ; CC(=O)OC[C@@H]1CCN1CCc1c[nH]c2ccccc12 ; O=P(O)(O)OC[C@@H]1CCN1CCc1c[nH]c2ccccc12 ; OCC1CCN1CCc1c(Br)[nH]c2ccccc12 ; OCC1CCN1CCc1c(Cl)[nH]c2ccccc12]

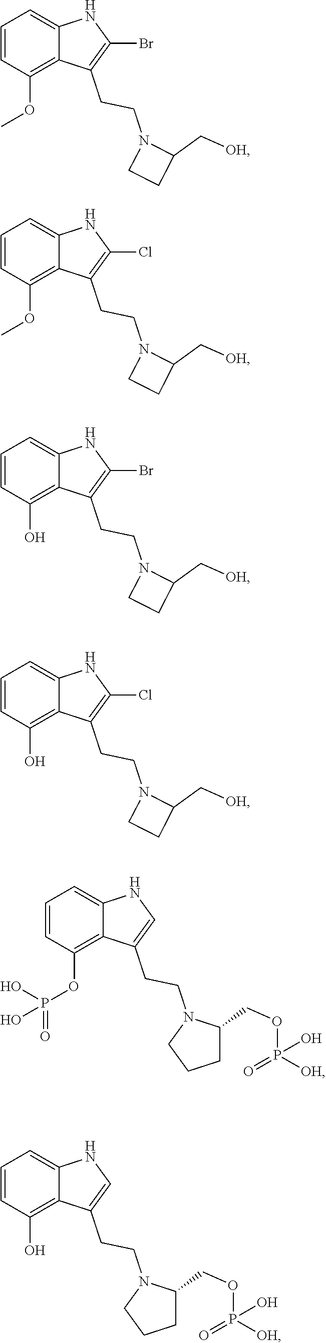 [SMILES: COc1cccc2[nH]c(Br)c(CCN3CCC3CO)c12 ; COc1cccc2[nH]c(Cl)c(CCN3CCC3CO)c12 ; OCC1CCN1CCc1c(Br)[nH]c2cccc(O)c12 ; OCC1CCN1CCc1c(Cl)[nH]c2cccc(O)c12 ; O=P(O)(O)OC[C@@H]1CCCN1CCc1c[nH]c2cccc(OP(=O)(O)O)c12 ; O=P(O)(O)OC[C@@H]1CCCN1CCc1c[nH]c2cccc(O)c12]

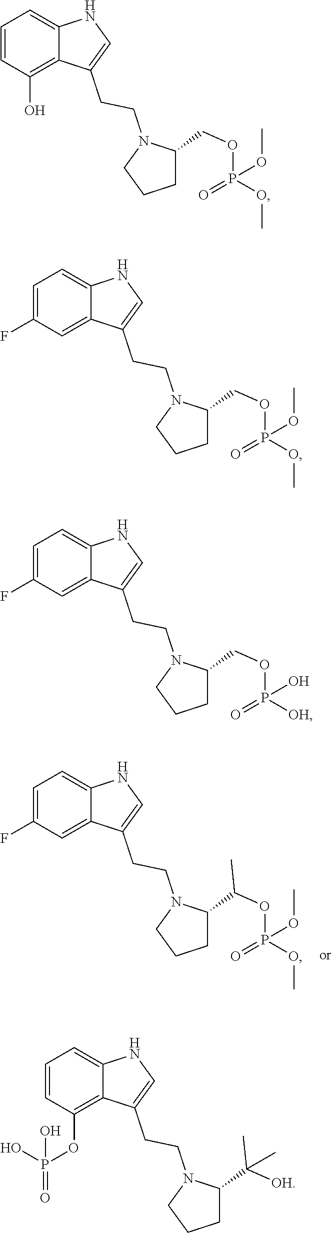 [SMILES: COP(=O)(OC)OC[C@@H]1CCCN1CCc1c[nH]c2cccc(O)c12 ; COP(=O)(OC)OC[C@@H]1CCCN1CCc1c[nH]c2ccc(F)cc12 ; O=P(O)(O)OC[C@@H]1CCCN1CCc1c[nH]c2ccc(F)cc12 ; COP(=O)(OC)OC(C)[C@@H]1CCCN1CCc1c[nH]c2ccc(F)cc12 ; "or" ; CC(C)(O)[C@@H]1CCCN1CCc1c[nH]c2cccc(OP(=O)(O)O)c12]

53. A compound of Formula (I-B) 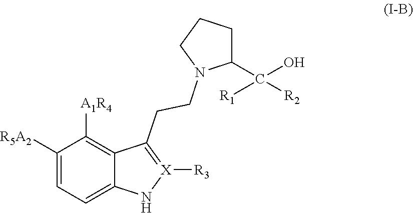
or a pharmaceutically acceptable salt or deuterated form thereof;
wherein,
A₁ and A₂ are each independently a bond, H, D, O, N, or S;
R₁ and R₂ are each independently H or alkyl;
R₃ is H, D, alkyl, cycloalkyl, aromatic, halogen, heteroaromatic, or absent;
R₄ and R₅ are each independently H, D, alkyl, cycloalkyl, aromatic, halogen substituted alkyl or cycloalkyl, halogen, or heteroaromatic, or
R₄ and R₅ are each independently a prodrug; and
X is independently —CH, —C, or —N.

54. The compound of claim 53, wherein R₁ is H.

55. The compound of claim 53, wherein R₁ is alkyl.

56. The compound of claim 53, wherein R₂ is H.

57. The compound of claim 53, wherein R₂ is alkyl.

58. The compound of claim 53, wherein A₁ is a bond.

59. The compound of claim 53, wherein A₁ is O.

60. The compound of claim 53, wherein A₂ is a bond.

61. The compound of claim 53, wherein A₂ is O.

62. The compound of claim 53, wherein R₃ absent.

63. The compound of claim 53, wherein R₃ is halogen.

64. The compound of claim 53, wherein R₃ is F, Cl, or Br.

65. The compound of claim 53, wherein R₄ is H.

66. The compound of claim 53, wherein R₄ is an alkyl.

67. The compound of claim 66, wherein the alkyl is —CH₃.

68. The compound of claim 53, wherein R₄ is a halogen substituted alkyl.

69. The compound of claim 68, wherein the halogen substituted alkyl is —CF₃.

70. The compound of claim 53, wherein R₄ is a halogen.

71. The compound of claim 70, wherein R₄ is F, Cl, or Br.

72. The compound of claim 53, wherein R₅ is H.

73. The compound of claim 53, wherein R₅ is an alkyl.

74. The compound of claim 73, wherein the alkyl is —CH₃.

75. The compound of claim 53, wherein R₅ is a halogen substituted alkyl.

76. The compound of claim 75, wherein the halogen substituted alkyl is —CF₃.

77. The compound of claim 53, wherein R₅ is a halogen.

78. The compound of claim 77, wherein R₅ is F, Cl, or Br.

79. The compound of claim 53, wherein at least one of R₄ or R₅ is a prodrug.

80. The compound of claim 79, wherein the prodrug is an aliphatic ester, aromatic ester, heteroaromatic ester, aminal, hemiaminal, —OPO₃H₂, —CH₂—OPO₃H₂, and —CH-alkyl-OPO₃H₂.

81. The compound of claim 1, wherein X is CH.

82. The compound of claim 81, wherein R₃ is absent.

83. The compound of claim 1, wherein X is —C.

84. The compound of claim 83, wherein R₃ is halogen.

85. The compound of claim 53, wherein A₁ is a bond and R₄ is H.

86. The compound of claim 53, wherein A₁ is a bond and R₄ is a halogen.

87. The compound of claim 53, wherein A₁ is a O and R₄ is an alkyl.

88. The compound of claim 87, wherein the alkyl is —CH₃.

89. The compound of claim 53, wherein A₁ is O and R₄ is a halogen substituted alkyl.

90. The compound of claim 89, wherein the halogen substituted alkyl is —CF₃.

91. The compound of claim 53, wherein A₁ is a bond and R₅ is H.

92. The compound of claim 53, wherein A₁ is a bond and R₅ is a halogen.

93. The compound of claim 53, wherein A₁ is a O and R₅ is an alkyl.

94. The compound of claim 93, wherein the alkyl is —CH₃.

95. The compound of claim 53, wherein A₁ is O and R₅ is a halogen substituted alkyl.

96. The compound of claim 95, wherein the halogen substituted alkyl is —CF₃.

97. The compound of claim 53, wherein the compounds is:

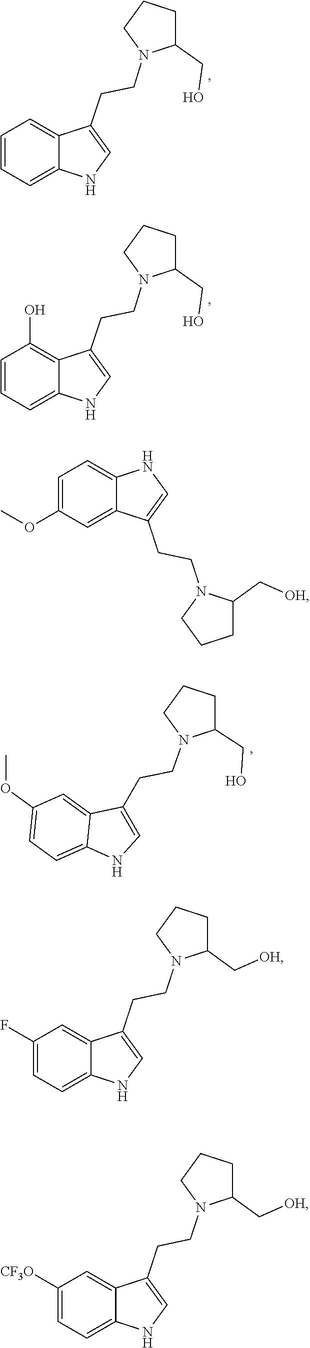

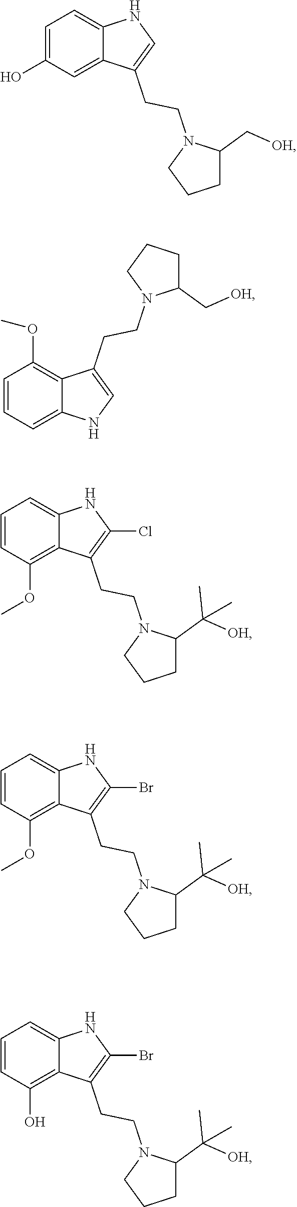 [SMILES: OCC1CCCN1CCc1c[nH]c2ccc(O)cc12 ; COc1cccc2[nH]cc(CCN3CCCC3CO)c12 ; COc1cccc2[nH]c(Cl)c(CCN3CCCC3C(C)(C)O)c12 ; COc1cccc2[nH]c(Br)c(CCN3CCCC3C(C)(C)O)c12 ; CC(C)(O)C1CCCN1CCc1c(Br)[nH]c2cccc(O)c12]

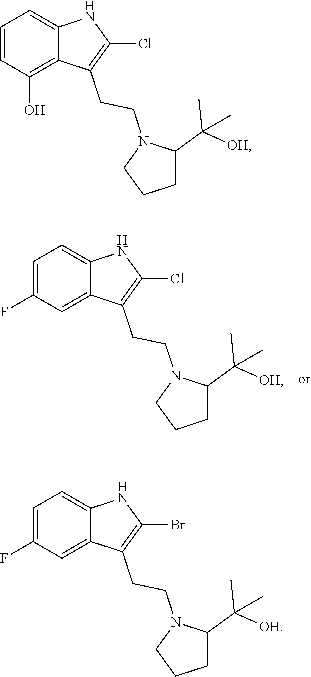 [SMILES: CC(C)(O)C1CCCN1CCc1c(Cl)[nH]c2cccc(O)c12 ; CC(C)(O)C1CCCN1CCc1c(Cl)[nH]c2ccc(F)cc12 ; "or" ; CC(C)(O)C1CCCN1CCc1c(Br)[nH]c2ccc(F)cc12]

98. The compound of claim 53, wherein the compounds is:

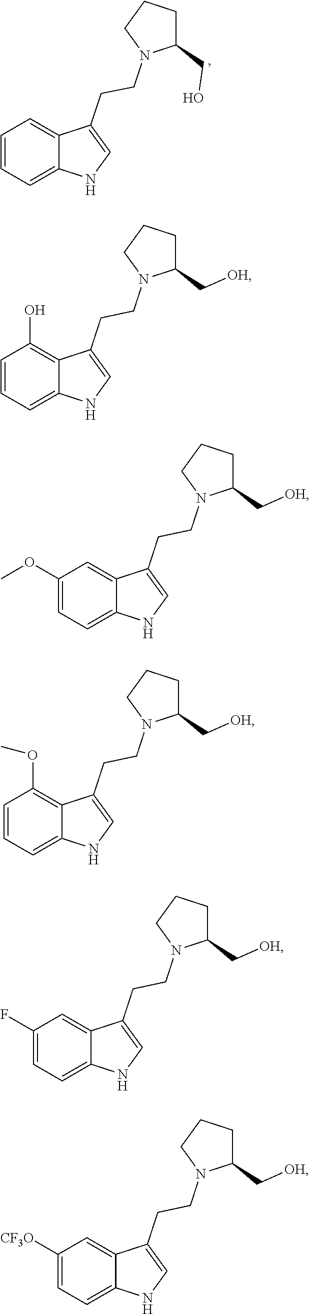

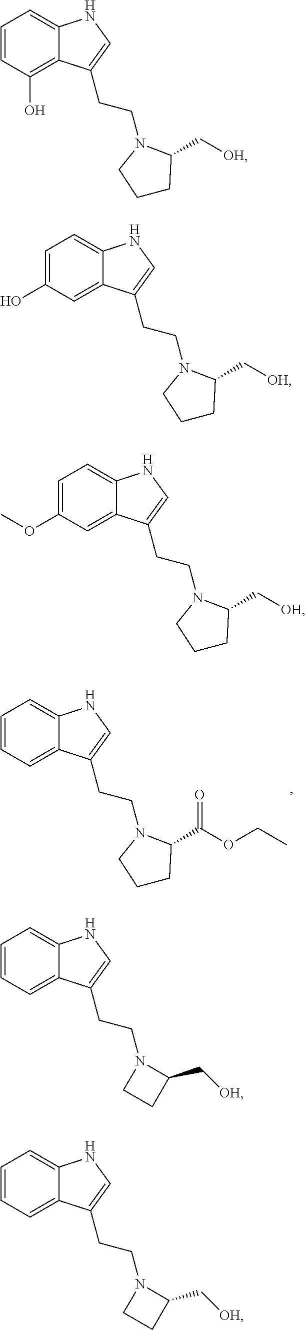

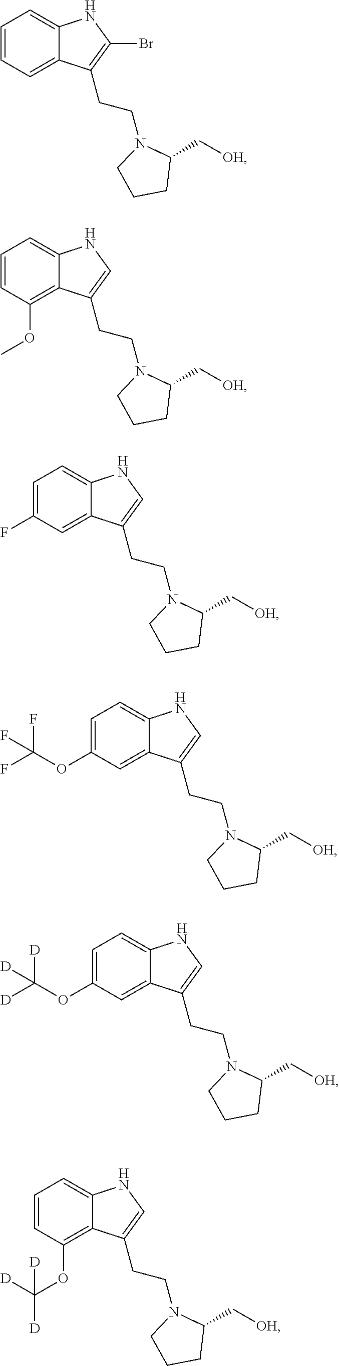 [SMILES: OC[C@@H]1CCCN1CCc1c(Br)[nH]c2ccccc12 ; COc1cccc2[nH]cc(CCN3CCC[C@H]3CO)c12 ; OC[C@@H]1CCCN1CCc1c[nH]c2ccc(F)cc12 ; OC[C@@H]1CCCN1CCc1c[nH]c2ccc(OC(F)(F)F)cc12 ; [2H]C([2H])([2H])Oc1ccc2[nH]cc(CCN3CCC[C@H]3CO)c2c1 ; [2H]C([2H])([2H])Oc1cccc2[nH]cc(CCN3CCC[C@H]3CO)c12]

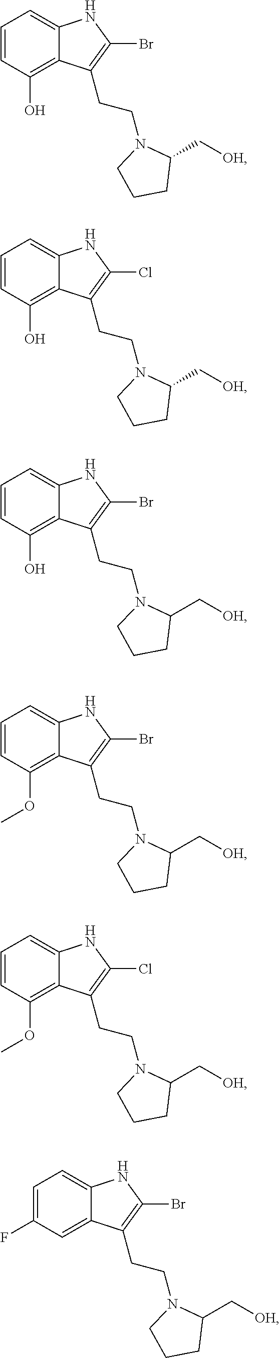 [SMILES: OC[C@@H]1CCCN1CCc1c(Br)[nH]c2cccc(O)c12 ; OC[C@@H]1CCCN1CCc1c(Cl)[nH]c2cccc(O)c12 ; OCC1CCCN1CCc1c(Br)[nH]c2cccc(O)c12 ; COc1cccc2[nH]c(Br)c(CCN3CCCC3CO)c12 ; COc1cccc2[nH]c(Cl)c(CCN3CCCC3CO)c12 ; OCC1CCCN1CCc1c(Br)[nH]c2ccc(F)cc12]

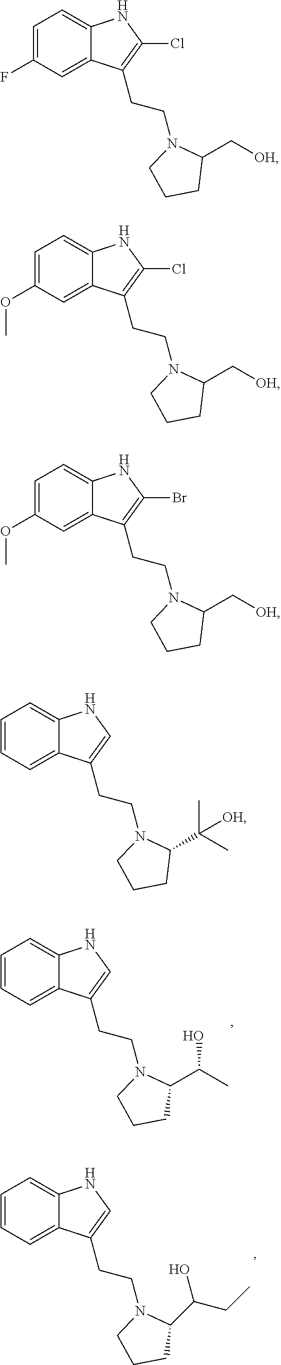 [SMILES: OCC1CCCN1CCc1c(Cl)[nH]c2ccc(F)cc12 ; COc1ccc2[nH]c(Cl)c(CCN3CCCC3CO)c2c1 ; COc1ccc2[nH]c(Br)c(CCN3CCCC3CO)c2c1 ; CC(C)(O)[C@@H]1CCCN1CCc1c[nH]c2ccccc12 ; C[C@@H](O)[C@@H]1CCCN1CCc1c[nH]c2ccccc12 ; CCC(O)[C@@H]1CCCN1CCc1c[nH]c2ccccc12]

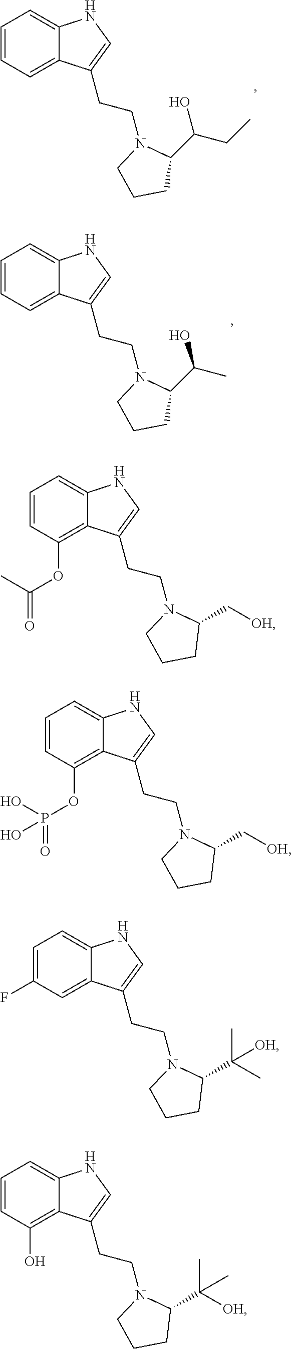

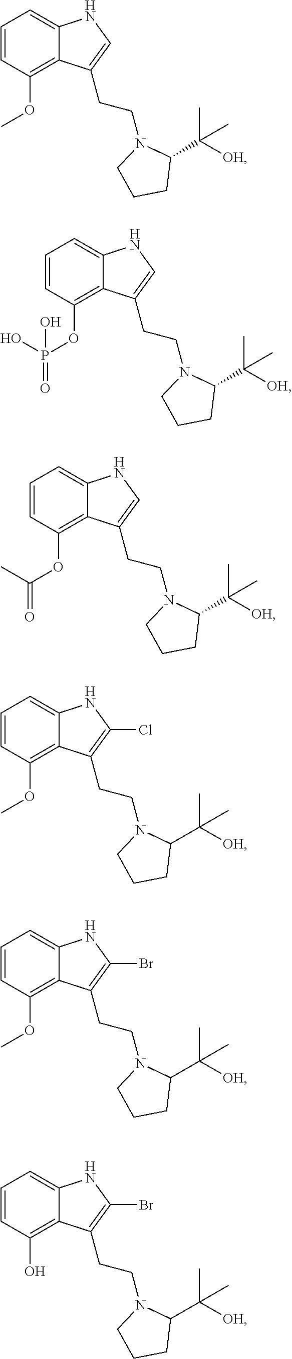

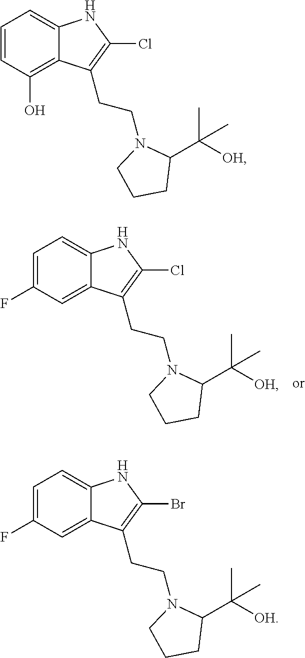 [SMILES: CC(C)(O)C1CCCN1CCc1c(Cl)[nH]c2cccc(O)c12 ; CC(C)(O)C1CCCN1CCc1c(Cl)[nH]c2ccc(F)cc12 ; "or" ; CC(C)(O)C1CCCN1CCc1c(Br)[nH]c2ccc(F)cc12]

99. A compound selected from:

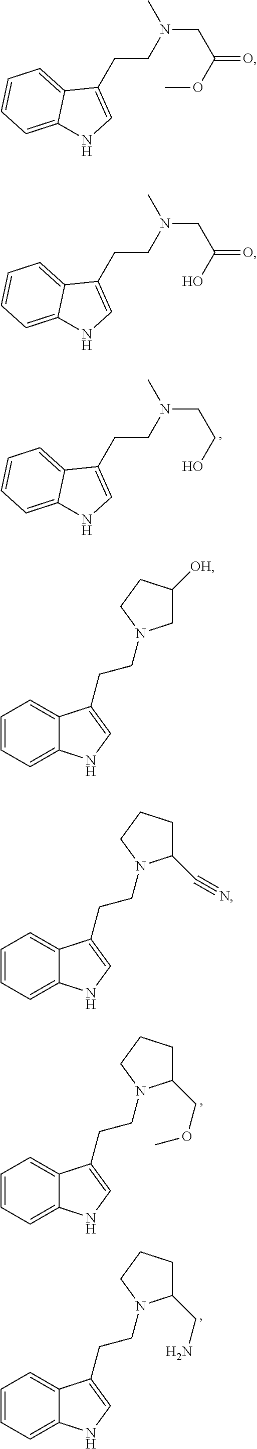

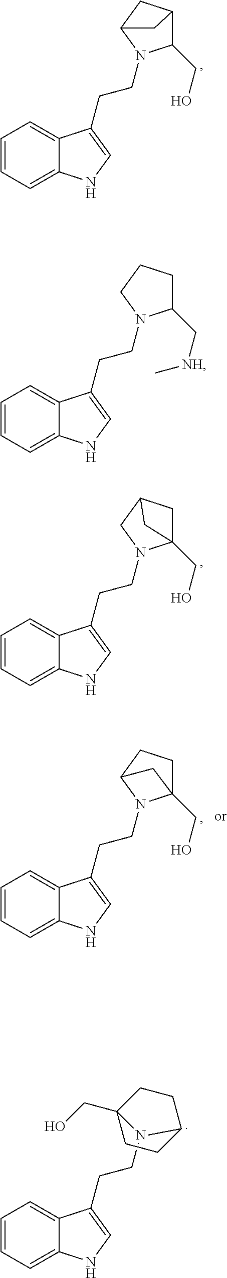

100. A pharmaceutical composition, comprising a compound of claim 1 and a pharmaceutically acceptable excipient.

101. A method of treating a mental health disease or disorder, the method comprising administering a therapeutically effective amount of a compound of compound of claim 1.
